(19) United States
(12) Patent Application Publication
(10) Pub. No.: US 2010/0266569 A1
(43) Pub. Date: Oct. 21, 2010

(54) PROSTATIC ACID PHOSPHATASE FOR THE TREATMENT OF PAIN

(76) Inventors: Mark Zylka, Chapel Hill, NC (US); Prikko Vihko, Helsinki (FI)

Correspondence Address: JENKINS, WILSON, TAYLOR & HUNT, P. A., Suite 1200 UNIVERSITY TOWER, 3100 TOWER BLVD.,, DURHAM, NC, 27707, US

(21) Appl. No.: 12/743,110

(22) PCT Filed: Nov. 17, 2008
(86) PCT No.: PCT/US08/12849

§ 371 Date: Jun. 29, 2010

Related U.S. Application Data
(60) Provisional application No. 61/003,205, filed on Nov. 15, 2007.

(51) Int. Cl. A61K 38/46; C12N 9/16; C07H 21/00; C12N 15/63; C12N 5/10; C12Q 1/68; G01N 33/50; A61P 25/00; A61P 25/04

(52) U.S. Cl. 424/94.6; 435/196; 536/23.2; 435/320.1; 435/325; 435/7.1; 435/6; 436/94

(57) ABSTRACT

Methods and compositions are provided for the treatment of pain and cystic fibrosis. The methods include administering to an animal a composition or a pharmaceutical formulation comprising a therapeutically effective amount of a Prostatic Acid Phosphatase (“PAP”) polypeptide, or an active variant, fragment or derivative thereof, or a therapeutically effective amount of an activity enhancing PAP modulator. PAP is provided as a treatment for chronic pain including neuropathic and inflammatory pain in animals and humans. The PAP, or the active variant, fragment or derivative thereof, or the activity enhancing modulator of the PAP is administered via one or more of injection, intrathecal injection, oral administration, a surgically implanted pump, stem cells, viral gene therapy, or naked DNA gene therapy. Intrathecal injection of PAP functions as an analgesic and reduces thermal sensitivity in mice. PAP can reduce chronic mechanical and thermal inflammatory pain in mice. Allodynia and hyperalgesia due to nerve injury can be prevented by increasing PAP activity in spinal cord.

### RELATED APPLICATIONS

This application claims the benefit of U.S. Provisional Patent Application Ser. No. 61/003,205, filed Nov. 15, 2007; the disclosure of which is incorporated herein by reference in its entirety.

### TECHNICAL FIELD

The presently disclosed subject matter pertains to the use of prostatic acid phosphatase (PAP) compositions for the treatment of pain.

### Abbreviations

° C.=degrees Celsius
μL=microliter
μmol=micromole
μU=microunit
ALP=alkaline phosphatase
AMP=adenosine monophosphate
BL=baseline
bPAP=bovine prostatic acid phosphatase
BSA=bovine serum albumin
CF=cystic fibrosis
CFA=complete Freund's adjuvant
CSF=cerbrospinal fluid
DEPC=diethylpyrocarbonate
DRG=dorsal root ganglia
FRAP=fluoride-resistant acid phosphatase
hPAP=human prostatic acid phosphatase
hr=hour
i.t.=intrathecal
LPA=lysophosphatidic acid
LTR=long terminal repeat
mg=milligram
MG=monoglyceride
mL=milliliter
mm=millimeter
mPAP=mouse prostatic acid phosphatase
mU=milliunit
nmol=nanomole
PAP=prostatic acid phosphatase
PBS=phosphate buffered saline
PEG=poly(ethylene glycol)
Pi=inorganic phosphate
s=second
SNI=spared nerve injury
SNP=single nucleotide polymorphism
TM-PAP=transmembrane prostatic acid phosphatase
w/v=weight to volume

### BACKGROUND

Pain affects more Americans than heart disease, diabetes and cancer combined. In fact, about 50 million Americans suffer from chronic pain and spend about $100 billion for treatments per year. Unfortunately, many of the strongest available analgesics have serious side-effects including addiction, dependence and increased risk of heart attack and stroke. Moreover, many chronic pain conditions cannot be effectively treated with existing medications. Considering the revenue of drugs like CELEBREX® ($2.8 billion in 2004; G.D.Searle & Co., Skokie, Ill., United States of America) and VIOXX® ($1.4 billion in 2004, Merck & Co., Inc., Whitehouse Station, N.J., United States of America), an effective treatment for chronic pain would significantly benefit human health. Accordingly, there is an unmet need for effective pain treatments.

### SUMMARY

In some embodiments, a method is provided for treating pain in an animal by administering a composition or a pharmaceutical formulation comprising a therapeutically effective amount of a PAP, or an active fragment, variant or derivative thereof, or a therapeutically effective amount of an activity enhancing PAP modulator. In some embodiments, all types of pain are treated including, but not limited to, pain characterized by one or more of: chronic pain, chronic inflammatory pain, neuropathic pain, chronic neuropathic pain, allodynia, hyperalgesia, nerve injury, trauma, tissue injury, inflammation, cancer, viral infection, Shingles, diabetic neuropathy, osteoarthritis, burns, joint pain or lower back pain, visceral pain, trigeminal neuralgia, migraine headache, cluster headache, headache, fibromyalgia and pain associated with childbirth.

In some embodiments, a method is provided for treating an animal for a disorder characterized at least in part by an excess of lysophosphatidic acid, comprising administering to the animal a composition or pharmaceutical formulation comprising a therapeutically effective amount of a PAP, or an active fragment, variant or derivative thereof, or a therapeutically effective amount of an activity enhancing PAP modulator.

In some embodiments, the animal is a human.

In some embodiments, the PAP is selected from the group consisting of human PAP, bovine PAP, rat PAP and mouse PAP, and active fragments, variants and derivatives thereof.

In some embodiments, the PAP or the active fragment, variant or derivative thereof, comprises one or more modifications selected from the group consisting of one or more: conservative amino acid substitutions; non-natural amino acid substitutions, D- or D,L-racemic mixture isomer form amino acid substitutions, amino acid chemical substitutions, carboxy- or amino-terminus modifications, conjugation to biocompatible molecules including fatty acids and PEG and conjugation to biocompatible support structures including agarose, sepharose and nanoparticles.

In some embodiments, the PAP is obtained by recombinant methods.

In some embodiments, the PAP or the activity enhancing modulator of the PAP is administered via one or more of injection, oral administration, a surgically implanted pump, stem cells, viral gene therapy, naked DNA gene therapy. In some embodiments, the injection is intravenous injection, epideral injection, or intrathecal injection. In some embodiments, the administration is via intrathecal injection of PAP-expressing embryonic stem cells. In some embodiments, the administration is by intrathecal injection about once every 3 days. In some embodiments, the administration is in combination with one or more of adenosine, adenosine monophosphate (AMP) or an AMP analogue. In some embodiments, the administration is in combination with a known analgesic. In some embodiments, the known analgesic is an opiate. In some embodiments, the administration is via viral gene therapy using a retroviral, adenoviral, or adeno-associated viral vector transfer cassette comprising a nucleic acid sequence encoding the PAP or active variant or fragment thereof.

In some embodiments, a method is provided for treating cystic fibrosis in an animal, the method comprising administering to the animal a composition or pharmaceutical formulation comprising a therapeutically effective amount of a PAP, or an active fragment, variant or derivative thereof, or a therapeutically effective amount of an activity enhancing PAP modulator. In some embodiments the administering is by aerosolizing in the lungs.

In some embodiments, a method is provided for increasing levels of adenosine in the lungs of an animal having a disorder characterized at least in part by a deficiency in adenosine or adenosine receptor function, the method comprising administering to the animal a composition or pharmaceutical formulation comprising a therapeutically effective amount of a PAP, or an active fragment, variant or derivative thereof, or a therapeutically effective amount of an activity enhancing PAP modulator.

In some embodiments, an isolated PAP peptide is provided. The peptide can be selected from the group consisting of human PAP, cow PAP, rat PAP and mouse PAP, and fragments, variants, and derivatives thereof. In some embodiments, an isolated nucleotide sequence is provided that encodes the PAP peptide. In some embodiments, an expression vector is provided that comprises the nucleotide sequence. In some embodiments, a host cell is provided that comprises the expression vector. In some embodiments, a retroviral, adenoviral, or adeno-associated viral vector transfer cassette is provided that comprises a nucleotide sequence encoding the PAP or active variant or fragment thereof.

In some embodiments, a composition is provided comprising the PAP peptide, or an active fragment, variant or derivative thereof, wherein the composition is prepared for administration to animals, or as a pharmaceutical formulation for administration to humans.

In some embodiments, a method is provided for screening for a small molecule modulator of PAP activity by measuring the activity of a PAP in the presence and absence of a candidate small molecule and identifying as PAP modulators the candidate small molecules that cause either an increase or a decrease in the PAP activity.

In some embodiments, a kit is provided for the treatment of pain in animals, comprising a composition or pharmaceutical formulation comprising a therapeutically effective amount of a PAP, or an active fragment, variant or derivative thereof, and a surgically implantable pump apparatus for delivery of PAP to local tissue.

In some embodiments, a method is provided for diagnosing an individual's response to a pain medicine, comprising identifying one or more single nucleotide polymorphisms (SNPs), insertions, deletions and/or other types of genetic mutations in and around a PAP genomic locus in the individual; and correlating the SNPs, insertions, deletions and/or other types of genetic mutations with a predetermined response to the pain medicine.

In some embodiments, a method is provided for diagnosing an individual's threshold for pain, comprising identifying one or more single nucleotide polymorphisms (SNPs) insertions, deletions and/or other types of genetic mutations in and around a PAP genomic locus in the individual; and correlating the SNPs, insertions, deletions and/or other types of genetic mutations with a predetermined threshold for pain.

In some embodiments, a method is provided for diagnosing an individual's propensity to transition from acute to chronic pain, comprising identifying one or more single nucleotide polymorphisms (SNPs) insertions, deletions and/or other types of genetic mutations in and around a PAP genomic locus in the individual; and correlating the SNPs, insertions, deletions and/or other types of genetic mutations with a predetermined threshold for pain.

In some embodiments, a method is provided for diagnosing an individual's response to a pain medication, threshold for pain or propensity to transition from acute to chronic pain, the method comprising correlating differences in PAP expression levels in the individual and a control population, and correlating the extent of differential expression with a predetermined response to a pain medication or a predetermined threshold for pain.

Accordingly, it is an object of the presently disclosed subject matter to provide methods and compositions for the treatment of pain and cystic fibrosis. These and other objects are achieved in whole or in part by the presently disclosed subject matter.

Objects of the presently disclosed subject matter having been stated above, other objects and advantages will become apparent upon a review of the following descriptions, figures and examples.

### BRIEF DESCRIPTION OF THE DRAWINGS

FIG. 1 is a schematic diagram depicting cells expressing the secreted and transmembrane isoforms of prostatic acid phosphatase (PAP). The catalytic site (active site) of PAP is located in the extra cellular space and in the lumen of vesicles (not shown). SP=signal peptide. TM=Transmembrane domain.

FIGS. 2A-2B are micrographs from in situ hybridization experiments with riboprobes complimentary to the unique 3′ untranslated regions of each prostatic acid phosphatase (PAP) isoform. FIG. 2A (left-hand micrograph) shows the PAP transmembrane isoform is expressed at high levels in mouse dorsal root ganglia (DRG) neurons. FIG. 2B (right-hand micrograph) shows the secreted isoform is expressed at low to undetectable levels. Scale bar=50 μm.

FIG. 3 is a set of bar graphs showing a fluorometric assay to quantify acid phosphatase activity. Left-hand Bar Graph: Pure bovine prostatic acid phosphatase (bPAP) protein purchased from Sigma (St Louis, Mo., United States of America). Right-hand Bar Graph: Mouse prostatic acid phosphatase (mPAP) assayed from transfected cell lysates. Activity is reduced by the PAP inhibitor L-tartrate (10 mM). These assays were performed following the manufacturers protocol (EnzChek Assay, Invitrogen, Carlsbad, Calif., United States of America) and quantified using a fluorescent microplate reader.

FIG. 4 is a graph showing bovine PAP (bPAP) inhibition of lysophosphatidic acid (LPA)-evoked signaling. Rat1 cells were loaded with the calcium sensitive indicator Fura2-AM and stimulated with LPA that was incubated for 1.5 hr at 37° C. with bPAP (see left side of graph under “a”).

After washout, the same cells were stimulated with LPA which was also incubated for 1.5 hr at 37° C., but without bPAP (see right side of graph under “b”). Average ratios from three independent experiments are plotted +/−SEM (in grey). n=60 cells in total were analyzed. The small error bars highlight the high degree of reproducibility between experiments.

FIG. 5 is a graph showing that Rat1 cells transfected with prostatic acid phosphatase (PAP)-Venus (light line) have smaller lysophosphatidic acid (LPA)-evoked calcium responses than untransfected cells (dark line) in the same field of view (average from 15 PAP+ and 15 untransfected cells; this was reproduced twice). This effect was not seen in cells transfected with Venus (not fused to PAP).

FIGS. 6A-6D are graphs showing that inhibition of lysophosphatidic acid (LPA)-evoked signaling by prostatic acid phosphatase (PAP) requires phosphatase activity. For FIGS. 6A and 6C (left-hand top and bottom graphs, respectively) Rat1 fibroblasts were transfected with wild-type mouse PAP (mPAP). For FIGS. 6B and 6D (right-hand top and bottom graphs, respectively) Rat1 fibroblasts were transfected with a phosphatase-dead PAP-mutant. Post-transfection, cells were loaded with the calcium-sensitive indicator Fura2-AM and stimulated with LPA. FIGS. 6A and 6B are plots showing Fura2 responses in untransfected cells or cells transfected with PAP constructs (visualized by Venus fluorescence). FIGS. 6C and 6D are bar graphs showing quantification of the area under the curve during 60 second LPA stimulation for untransfected cells (shaded pars) and cells transfected with PAP constructs (open bars). Statistics: unpaired t test. Note that the absolute area in FIG. 6C and FIG. 6D differ due to variability in loading dishes of cells on different days with fura2.

FIG. 7 is a schematic diagram showing how peripheral nerve injury causes neuropathic pain that is dependent on lysophosphatidic acid (LPA) receptor signaling. Prostatic acid phosphatase (PAP) dephosphorylates LPA to monoglyceride (MG) and inorganic phosphate (Pi). PAP is down-regulated in dorsal root ganglia (DRG) neurons post injury.

FIGS. 8A-8C are graphs showing neuropathic pain behavior. FIG. 8A (left-hand graph) shows that injury to peripheral nerves causes allodynia and hyperalgesia during Initiation phase (Ini; shaded dark grey), which persists during Maintenance phase (shaded light grey). FIG. 8B (center graph) shows that injection of soluble prostatic acid phosphatase (PAP) before nerve injury can block initiation. FIG. 8C (right-hand graph) shows that injection of PAP after nerve injury is analgesic during maintenance phase.

FIG. 9 is a schematic diagram showing that neuropathic pain can be treated by increasing lysophosphatidic acid (LPA) phosphatase activity. Prostatic acid phosphatase (PAP) degrades LPA and reduces LPA-evoked signaling. Several methods (a-d) exist for increasing PAP in the nociceptive system.

FIGS. 10A-10B are graphs showing bovine prostatic acid phosphatase (bPAP) inhibition of lysophosphatidic acid (LPA)-evoked sensitization in vivo. Mechanical (FIG. 10A, graph on the left) and noxious thermal (FIG. 10B, graph on the right) sensitivity of wild-type C57BL/6 male mice before (baseline; BL) and after i.t. injection of vehicle (black-solid line), 20 μU bPAP (black-dashed line), 1 nmol LPA (gray-dashed line) or 1 nmol LPA+20 μU bPAP (gray-solid line). All samples were incubated at 37° C. for 10 min prior to injection. Injection volume: 5 μL. N=5 mice per condition. Error bars: +/−SEM. Statistics: unpaired t-test relative to vehicle. p<0.05 (*); p<0.005 (**); p<0.0005 (***).

FIGS. 11A-11D are graphs showing that bovine prostatic acid phosphatase (bPAP) and human prostatic acid phosphatase (hPAP) are analgesic in vivo. Noxious thermal (FIGS. 11A and 11C) and mechanical (FIGS. 11B and 11D) sensitivity of wild-type C57BL/6 male mice before (baseline; BL) and after i.t. injection of vehicle (solid line, FIGS. 11A and 11B) or BSA (solid line, FIG. 11C and 11D) or 20 μU bPAP (dashed line, FIGS. 11A and 11B) or 1.3 mg/mL hPAP (dashed line, FIGS. 11C and 11D). Injection volume: 5 μL. N=5 mice per condition. Error bars: ±SEM. Statistics: unpaired t-test relative to vehicle. p<0.05 (*); p<0.005 (**).

FIGS. 12A-12B are graphs showing the effect of bovine alkaline phosphatase (ALP) on noxious thermal (FIG. 12A) and mechanical (FIG. 12B) sensitivity of wild-type C57BL/6 mice before (baseline; BL) and after i.t. injection with recombinant ALP (arrow; 5000 U/mL; 25,000 mU total). The unit definition for PAP and ALP is essentially the same (1 U will hydrolyze 1 pmole of 4-nitrophenyl phosphate per minute at 37° C. at pH 4.8 or pH 9.8, respectively). Thus, 25,000 mU ALP has 100 times more phosphatase activity than the 250 mU hPAP used to provide the data shown in FIG. 13, described below. Paired t-tests were used to compare responses at each time point to baseline values. There were no significant differences at any of the time points in these assays. All data are presented as means±SEM (some of the error bars are obscured due to their small size). When a lower concentration of ALP (250 mU, i.t.) was used, it was also found not to reduce thermal or mechanical sensitivity (data not shown).

FIG. 13 is a graph showing that intrathecal injection of active human prostatic acid phosphatase (hPAP, 250 mU) causes analgesia to noxious thermal stimuli in mice. Increased paw withdrawal latency is indicative of analgesia. Increased paw withdrawal latency is not observed in mice treated with inactive hPAP. Thermal sensitivity of wild-type C57BL/6 male mice is shown before (baseline is at time 0) and for 6 days post i.t. injection of active hPAP (solid line) or inactive hPAP (dashed line). Injection volume: 5 μL. N=10 mice per condition. Statistics: Unpaired t-test relative to inactive hPAP. Error bars: +/−SEM.

FIGS. 14A-14C are graphs showing the dose dependence of intrathecal injection of human prostatic acid phosphatase (hPAP). The top graph, FIG. 14A shows the dose dependency of i.t. injection of inactive hPAP (shaded circles) or increasing amounts (0.25 mU, shaded squares; 2.5 mU, shaded triangles; 25 mU, dark circles; or 250 mU, dark squares) of active hPAP on paw withdrawal latency to a radiant heat source. FIG. 14B shows the same data plotted as area under the curve {AUC; units are in Latency (s)×Time post injection (h); integrated over 72 h (3 days) post injection} relative to mice injected with inactive PAP. FIG. 14B, inset, is the data plotted on log scale. FIG. 14C is a graph of the data from the two day time points plotted as percent maximal increase in paw withdrawal latency relative to baseline (BL). FIG. 14C, inset, is the two day time point data plotted on log scale. Injection volume: 5 μL. N=8 wild-type C57BL/6 male mice for the 0.25 mU, 2.5 mU, and 25 mU amounts; N=24-74 wild-type C57BL/6 male mice for the inactive hPAP and 250 mU amounts. Curves were generated by non-linear regression analysis using Prism 5.0 (GraphPad™ Software, Inc., La Jolla, Calif., United States of America). Error bars: +/−SEM. Significant differences are shown relative to baseline (paired t-tests); * P<0.05; ** P<0.005; *** P<0.0005.

FIG. 15 is a graph showing that mechanical sensitivity in mice is unchanged after treatment with intrathecal injection of active human prostatic acid phosphatase (hPAP, 250 mU). Thermal sensitivity of wild-type C57BL/6 male mice is shown before (baseline is at time 0) and for 6 days post i.t.

injection of active hPAP (solid line) or inactive hPAP (dashed line). Injection volume: 5 μL. N=10 mice per condition. No significant differences at any time point. Error bars: +/−SEM.

FIGS. 16A-16C are graphs showing the dose-dependent anti-nociceptive effects of intrathecal morphine sulfate. The top graph, FIG. 16A shows the dose dependency of i.t. injection of vehicle (shaded circles) or increasing amounts (0.01 μg, dark squares; 0.1 μg, triangles; 1 μg, circles; 10 μg, shaded squares; 50 μg, dark circles) of morphine sulfate (Morphine/V-arrow) on paw withdrawal latency to a radiant heat source. Side-effects were observed at the two highest doses. At the 10 μg dose three mice were paralyzed and displayed a Straub tail lasting 3-5 h. At the 50 μg dose two mice died while three other mice were paralyzed and displayed a Straub tail lasting 1-2 h. Straub tail is visualized as a stiff tail held above horizontal (Hylden and Wilcox, 1980). High doses of i.t. morphine are known to cause motor impairment and lethality (Dirig and Yaksh, 1995; Grant et al., 1995; Nishiyama et al., 2000). FIG. 16B shows the same data plotted as area under the curve {AUC; units are in Latency (s)×Time post injection (h); integrated over entire time course} relative to mice injected with vehicle. FIG. 16B, inset, shows the data plotted on log scale. FIG. 16C shows the data from the 1 h time points plotted as percent maximal increase in paw withdrawal latency relative to baseline (BL). FIG. 16C, inset, shows the 1 h time point data plotted on log scale. Injection (i.t.) volume was 5 μL. n=8 wild-type mice were used per dose. Curves were generated by non-linear regression analysis using Prism 5.0 (GraphPad™ Software, Inc., La Jolla, Calif., United States of America). Significant differences are shown relative to baseline (paired t-tests); * P<0.05; ** P<0.005; *** P<0.0005. All data are presented as means±SEM.

FIGS. 17A-17B are graphs showing that bovine prostatic acid phosphatase (bPAP) is analgesic in the Complete Freund's Adjuvant (CFA) model of inflammatory pain in mice. Noxious thermal (FIG. 17A) and mechanical (FIG. 17B) sensitivity of wild-type C57BL/6 male mice are shown before (baseline; BL), 1 day after CFA injection into hindpaw, and after i.t. injection of BSA (solid line) or 20 μU bPAP (dashed line). Injection volume: 5 μL. N=5 mice per condition. Error bars: ±SEM. Statistics: unpaired t-test relative to vehicle. p<0.05 (*).

FIG. 18 is a graph showing that human prostatic acid phosphatase (hPAP) is analgesic in the Complete Freund's Adjuvant (CFA) model of inflammatory pain in mice. Thermal sensitivity of CFA injected or uninjected hindpaws of wild-type C57BL/6 male mice is shown after i.t. injection of either active (injected paw, heavy solid line; uninjected paw, light solid line) or inactive hPAP (injected paw, heavy dashed line; uninjected paw, light dashed line). Active hPAP reduces thermal sensitivity in both CFA treated and untreated paws relative to inactive hPAP.

FIG. 19 is a graph showing that human prostatic acid phosphatase (hPAP) is analgesic in the Complete Freund's Adjuvant (CFA) model of inflammatory pain in mice. Mechanical sensitivity of CFA injected or uninjected hindpaws of wild-type C57BL/6 male mice is shown after i.t. injection of either active (injected paw, heavy solid line; uninjected paw, light solid line) or inactive hPAP (injected paw, heavy dashed line; uninjected paw, light dashed line). Active hPAP reduces mechanical sensitivity relative to inactive PAP in CFA-injected paws only. N=10 mice tested.

FIG. 20 is a graph showing that bovine prostatic acid phosphatase (bPAP) is analgesic in the Spared Nerve Injury (SNI) model of neuropathic pain in mice. Noxious thermal sensitivity of injured (left paw, shaded squares) or uninjured (right paw, open diamonds) hindpaws of wild-type C57BL/6 male mice is shown after i.t. injection of active bPAP. A reduction in thermal sensitivity is observed for both injured and uninjured paws for about 3 days following bPAP injection. N=7 mice tested.

FIG. 21 is a graph showing that bovine prostatic acid phosphatase (bPAP) is analgesic in the Spared Nerve Injury (SNI) model of neuropathic pain in mice. Mechanical sensitivity of injured left (shaded squares) or uninjured right (open diamonds) hindpaws of wild-type C57BL/6 male mice is shown after i.t. injection of active bPAP. A reduction in mechanical sensitivity is observed for injured but not uninjured paws for about 3 days following bPAP injection. N=7 mice tested.

FIG. 22 is a graph showing that human prostatic acid phosphatase (hPAP) is analgesic in the Spared Nerve Injury (SNI) model of neuropathic pain in mice. Thermal sensitivity of injured or uninjured hindpaws of wild-type C57BL/6 male mice is shown after i.t. injection of active (injured paw, shaded squares; uninjured paw, open squares) or inactive hPAP (injured paw, shaded triangles; uninjured paw, open triangles). A reduction in thermal sensitivity is observed for both injured and uninjured paws for about 3 days following active hPAP injection.

FIG. 23 is a graph showing that human prostatic acid phosphatase (hPAP) is analgesic in the Spared Nerve Injury (SNI) model of neuropathic pain in mice. Mechanical sensitivity of injured or uninjured hindpaws of wild-type C57BL/6 male mice is shown after i.t. injection of active (injured paw, shaded squares; uninjured paw, open squares) or inactive hPAP (injured paw, shaded triangles; uninjured paw, open triangles). A reduction in mechanical sensitivity is observed for injured but not uninjured paws for about 3 days following active hPAP injection.

FIGS. 24A-24D are graphs showing that PAP^{-/-} mice display enhanced nociceptive responses in the Complete Freund's Adjuvant (CFA) model of inflammatory pain (FIGS. 24A and 24B) and in the Spared Nerve Injury (SNI) model of neuropathic pain (FIGS. 24C and 24D). Wild-type and PAP^{-/-} mice were tested for (FIG. 24A) thermal sensitivity using a radiant heat source and (FIG. 24B) mechanical sensitivity using an electronic von Frey semi-flexible tip before (baseline, BL) and following injection of CFA (CFA-arrow) into one hindpaw (wild-type mice, open circles; PAP^{-/-} mice, dark squares). The non-inflamed hindpaw (wild type mice, gray circles; PAP^{-/-} mice, gray squares) served as control. For the SNI model, the sural and common peroneal branches of the sciatic nerve were ligated then transected (Injure-arrow). Injured (wild-type mice, open circles; PAP^{-/-} mice, dark squares) and non-injured (control; wild-type mice, grey circles; PAP^{-/-} mice, grey squares) hindpaws were tested for (FIG. 24C) thermal and (FIG. 24D) mechanical sensitivity. Paired t-tests were used to compare responses at each time point between wild-type (n=10) and PAP^{-/-} mice (n=10); same paw comparisons. * P<0.05; ** P<0.005; *** P<0.0005. All data are presented as means±SEM.

FIGS. 25A-25B are graphs showing the nociceptive effects of intraspinal prostatic acid phosphatase (PAP) in PAP^{-/-} mice and PAP rescue of chronic inflammatory pain behavioral phenotype in PAP^{-/-} mice. Wild-type (WT) and PAP^{-/-} (PAP KO) mice were tested for (FIG. 25A) thermal sensitivity and (FIG. 25B) mechanical sensitivity before (baseline, BL) and following injection of Complete Freund's Adjuvant (CFA-arrow) into one hindpaw (i.e., the left hindpaw). The non-inflamed (right) hindpaw served as control. One day later, half of the wild-type and PAP^{-/-} mice were injected with active human PAP (hPAP-arrow; 250 mU, i.t.) while the other half were injected with inactive hPAP. Data from these inactive hPAP injected mice were presented in FIGS. 24A and 24B, described above. In FIG. 25A, the data for the wild-type contol paw is shown with lightly shaded circles, for the wild type inflamed paw with darkly shaded circles, for wild-type control paw with active hPAP in lightly shaded triangles, for wild-type inflamed paw with active hPAP with unshaded triangles, for PAP KO contol paw with lightly shaded squares, for the PAP KO inflamed paw with darkly shaded squares, for the PAP KO control paw with active PAP with lightly shaded diamonds, and for the PAP KO inflamed paw with active PAP with unshaded diamonds. For FIG. 25B, the data for the wild-type contol paw is shown with lightly shaded circles, for the wild type inflamed paw with unshaded circles, for wild- type control paw with active hPAP in darkly shaded diamonds, for wild-type inflamed paw with active hPAP with unshaded diamonds, for PAP KO contol paw with unshaded squares, for the PAP KO inflamed paw with darkly shaded squares, for the PAP KO control paw with active PAP with lightly shaded triangles, and for the PAP KO inflamed paw with active PAP with unshaded triangles Paired t-tests were used to compare responses at each time point between wild-type (n=10/group) and PAP^{-/-} mice (n=10/group); same paw comparisons (n=40 mice were used for this experiment). * P<0.05; ** P<0.005; *** P<0.0005. All data are presented as means±s.e.m.

FIGS. 26A-26H show data related to prostatic acid phosphatase (PAP) ecto-5′-nucleotidase activity as revealed by dephosphorylation of adenosine monophosphate (AMP) to adenosine in vitro, in cells and in nociceptive circuits. FIG. 26A is a graph showing the effects of human prostatic acid phosphatase (hPAP, 2.5 U/mL) on 1 mM AMP, adenosine diphosphate (ADP), or adenosine triphosphate (ATP) as measured by increase in adenosine concentration. Dephosphorylation reactions (n=3 per time point) were stopped by heat denaturation at the indicated times. Conversion of nucleotides to adenosine was measured by high performance liquid chromatograph (HPLC). Data are presented as means±SEM. FIG. 26B shows the HPLC chromatogram before (t=0) and after (t=240 min) incubation of 1 mM AMP with human prostatic acid phosphatase (hPAP). Peaks corresponding to adenosine (ado) and AMP are indicated. Arbitrary units (a.u.). FIGS. 26C and 26D are micrographs showing HEK 293 cells transfected with a mouse transmembrane PAP (TM-PAP) expression construct (FIG. 26C) or with empty pcDNA3.1 vector (FIG. 26D) and then stained using AMP histochemistry. The plasma membrane was not permeabilized so that extracellular phosphatase activity could be assayed. FIGS. 26E-26H are micrographs showing lumbar dorsal root ganglia (DRG; FIGS. 26E and 26H) and spinal cord (FIGS. 26G-26H) from wild-type (FIGS. 26E and 26G) and PAP^{-/-} (FIGS. 26F and 26H) adult mice stained using AMP histochemistry. Motor neurons in the ventral horn of wild type and PAP^{-/-} spinal cord were also stained. Identical results were obtained from five additional mice of each genotype. AMP (6 mM in FIGS. 26C and 26D and 0.3 mM in FIGS. 26E-26H) was used as substrate and buffer pH was 5.6. Scale bar: 50 μm in FIGS. 26C-26F; 500 μm in FIGS. 26G and 26H.

FIGS. 27A-27F are graphs showing that prostatic acid phosphatase (PAP) requires A₁-adenosine receptors for anti-nociception. Wild-type (open circles) and A₁R^{-/-} (dark squares) mice were tested for thermal (FIG. 27A) and mechanical (FIG. 27B) sensitivity before (baseline, BL) and following i.t. injection of human prostatic acid phosphatase (hPAP-arrow). Complete Freund's Adjuvant (CFA) was injected into one hindpaw (CFA-arrow) of wild-type and A₁R^{-/-} mice. Active or inactive human prostatic acid phosphatase (hPAP) was i.t. injected one day later (hPAP-arrow). Inflamed (wild-type mice, open circles; A₁R^{-/-} mice, dark squares) and non-inflamed (control; wild-type mice, shaded circles; A₁R^{-/-} mice, shaded squares) hindpaws were tested for thermal (FIG. 27C) and mechanical (FIG. 27D) sensitivity. The Spared Nerve (SNI) model was used to induce neuropathic pain (Injure-arrow) in wild-type and A₁R^{-/-} mice. Active or inactive hPAP was i.t. injected four days later (hPAP-arrow). Injured (wild-type mice, open circles; A₁R^{-/-} mice, dark squares) and non-injured (control; wild-type mice, shaded circles; A₁R^{-/-} mice, shaded squares) hindpaws were tested for thermal (FIG. 27E) and mechanical (FIG. 27F) sensitivity. For all experiments, 250 mU hPAP was injected per mouse. T-tests were used to compare responses at each time point between wild-type (n=10) and A₁R^{-/-} mice (n=9); same paw comparisons. * P<0.05; ** P<0.005; *** P<0.0005. All data are presented as means±SEM.

FIGS. 28A-28B are graphs showing that A₁-adenosine receptors (A₁R) are required for bovine prostatic acid phsophatase (bPAP) anti-nociception. Wild-type mice (open circles, n=7) and A₁R^{-/-} mice (dark squares, n=7) were tested for thermal (FIG. 28A) and mechanical (FIG. 28B) sensitivity before (baseline, BL) and following i.t. injection of active bPAP (0.3 U/mL; arrow). Paired t-tests were used to compare responses at each time point between wild-type and knockout mice. Significant differences are shown; * P<0.05; ** P<0.005; *** P<0.0005. All data are presented as means±SEM.

FIGS. 29A-29B are graphs showing that the anti-nociceptive effects of prostatic acid phosphatase (PAP) can be transiently inhibited with a selective A₁-adenosine receptor (A₁R) antagonist. Wild-type mice were tested for noxious thermal (FIG. 29A) and mechanical (FIG. 29B) sensitivity before (baseline, BL) and following injection of Complete Freund's Adjuvant (CFA-arrow) into one hindpaw (inflamed paw, open circles or dark squares). The non-inflamed hindpaw served as control (shaded circles or squares). All mice were injected with active hPAP (hPAP-arrow; 250 mU, i.t.). Two days later, half the mice were injected with vehicle (CPXN-arrow, circles; intraperitoneal (i.p.); 1 h before behavioral measurements) while the other half were injected with 8-cyclopentyl-1,3-dipropylxanthine (CPX/V-arrow, squares; 1 mg/kg i.p.; 1 h before behavioral measurements). CPX transiently antagonized all anti-nociceptive effects of hPAP. In contrast, CPX did not affect thermal or mechanical sensitivity when injected on day 9, four days after the anti-nociceptive effects of hPAP wore off. Paired t-tests were used to compare responses at each time point between vehicle (n=10) and CPX-injected mice (n=10); same paw comparisons. *** P<0.0005. All data are presented as means±SEM.

FIGS. 30A-30C are graphs showing the dose-dependent anti-nociceptive effects of intrathecal N⁶-cyclopentyladenosine (CPA), a selective A₁-adenosine receptor (A₁R) agonist. FIG. 30A shows the effects of injecting (i.t.) vehicle or increasing doses (0.0005 nmol-5 nmol) of CPA (CPA/V-arrow) on paw withdrawal latency to the radiant heat source. Almost all mice injected with the two highest doses of CPA reached the cutoff of 20 s because of fore- and hindlimb paralysis lasting one hour (boxed region). High doses of adenosine receptor agonists are known to cause motor paralysis (Sawynok, 2006). FIG. 20B shows the same data as for FIG. 30A plotted as area under the curve {AUC; units are in Latency (s)×Time post injection (h); integrated over entire time course} relative to mice injected with vehicle. FIG. 30B, inset shows the data plotted on log scale. FIG. 30C shows the data from the 1 h time points plotted as percent maximal increase in paw withdrawal latency relative to baseline (BL). FIG. 30C, inset, shows the data from the 1 h time points plotted on log scale. Injection (i.t.) volume was 5 pt. n=8 wild-type mice were used per dose. All data are presented as means±s.e.m. Curves were generated by non-linear regression analysis using Prism 5.0 (GraphPad™ Software, Inc., La Jolla, Calif., United States of America). Significant differences are shown relative to baseline (paired t-tests); * P<0.05; ** P<0.005; *** P<0.0005. All data are presented as means±SEM.

### DETAILED DESCRIPTION

In accordance with the presently disclosed subject matter, methods and compositions are provided for the treatment of pain and cystic fibrosis. In some embodiments, the protein called Prostatic Acid Phosphatase (PAP) is provided for the treatment of these disorders. PAP protein is highly effective at treating chronic inflammatory and neuropathic pain in animal models when injected intrathecally (into spinal cord). A single injection of PAP protein can produce analgesia for up to three days. Such a single administration that relieves pain for three days is a vast improvement over existing pain treatments.

### I. Definitions

While the following terms are believed to be well understood by one of ordinary skill in the art, the following definitions are set forth to facilitate explanation of the presently disclosed subject matter.

Following long-standing patent law convention, the terms “a” and “an” mean “one or more” when used in this application, including the claims.

Unless otherwise indicated, all numbers expressing quantities of ingredients, reaction conditions, and so forth used in the specification and claims are to be understood as being modified in all instances by the term “about”. Accordingly, unless indicated to the contrary, the numerical region of parameters set forth in this specification and attached claims are approximations that can vary depending upon the desired properties sought to be obtained by the presently disclosed subject matter.

As used herein, the term “animal” refers to any animal (e.g., an animal), including, but not limited to, humans, non-human primates, rodents, and the like, which is to be the recipient of a particular treatment.

“Amino acid sequence” and terms such as “peptide”, “polypeptide” and “protein” are used interchangeably herein, and are not meant to limit the amino acid sequence to the complete, native amino acid sequence (i.e. a sequence containing only those amino acids found in the protein as it occurs in nature) associated with the recited protein molecule. The proteins and protein fragments of the presently disclosed subject matter can be produced by recombinant approaches or can be isolated from a naturally occurring source.

Similarly, all genes, gene names, and gene products disclosed herein are intended to correspond to homologs from any species for which the compositions and methods disclosed herein are applicable. Thus, the terms include, but are not limited to genes and gene products from humans and mice. It is understood that when a gene or gene product from a particular species is disclosed, this disclosure is intended to be exemplary only, and is not to be interpreted as a limitation unless the context in which it appears clearly indicates. Thus, for example, for the genes disclosed herein, which in some embodiments relate to mammalian nucleic acid and amino acid sequences by GENBANK® Accession No., are intended to encompass homologous and/or orthologous genes and gene products from other animals including, but not limited to other mammals, fish, amphibians, reptiles, and birds.

The term “LPA” stands for lysophosphatidic acid.

A “modulator” of PAP is referring to a small molecule that can modulate PAP catalytic activity. PAP modulators can be either activators or inhibitors of PAP activity.

The term “PAP” means a protein having prostatic acid phosphatase activity (E.C. 3.1.3.2.). The term “ACPP” (i.e., acid phosphatase, prostate) is herein used interchangeably with “PAP”. The GENBANK® database discloses amino acid and nucleic acid sequences of PAPs from various species, some of which are summarized in Table 1, below.

TABLE 1
GENBANK ® Accession Nos. for PAP
Amino Acid and Nucleic Acid Sequences
from Representative Species

| | | GENBANK ® Accession Nos. | |
|---|---|---|---|
| Species | Form | Nucleic Acid | Amino Acid |
| H. sapiens | transmembrane | NM_001134194 | NP_001127666 |
| H. sapiens | secreted | NM_001099 | NP_001090 |
| M. musculus | transmembrane | NM_207668 | NP_997551 |
| M. musculus | secreted | NM_019807 | NP_062781 |
| B. taurus | | NM_001098866 | NP_001092336 |
| R. norvegicus | transmembrane | NM_001134901 | NP_001128373 |
| R. norvegicus | secreted | NM_020072 | NP_064457 |

A “recombinant expression cassette” or simply an “expression cassette” is a nucleic acid construct, generated recombinantly or synthetically, with nucleic acid elements which permit transcription of a particular nucleic acid in a cell. The recombinant expression cassette can be part of a plasmid, virus, or other vector. Typically, the recombinant expression cassette includes a nucleic acid to be transcribed, a promoter, and/or other regulatory sequences. In some embodiments, the expression cassette also includes, e.g., an origin of replication, and/or chromosome integration elements (e.g., a retroviral LTR).

A “retrovirus” is a single stranded, diploid RNA virus that replicates via reverse transcriptase and a retroviral virion. A retrovirus can be replication-competent or replication incompetent. The term “retrovirus” refers to any known retrovirus (e.g., type c retroviruses, such as Moloney murine leukemia virus (MoMuLV), Harvey murine sarcoma virus (HaMuSV), murine mammary tumor virus (MuMTV), gibbon ape leukemia virus (GaLV), feline leukemia virus (FLV) and Rous Sarcoma Virus (RSV). “Retroviruses” of the presently disclosed subject matter also include human T cell leukemia viruses, HTLV-1 and HTLV-2, and the lentiviral family of retroviruses, such as, but not limited to, human immunodeficiency viruses HIV-1 and HIV-2, simian immunodeficiency virus (SIV), feline immunodeficiency virus (FIV), and equine immnodeficiency virus (EIV).

Several terms herein can be used interchangeably. Thus, “virion”, “virus”, “viral particle”, “viral vector”, “viral construct”, “vector particle”, “viral vector transfer cassette” and “shuttle vector” can refer to virus and virus-like particles that are capable of introducing nucleic acid into a cell through a viral-like entry mechanism. Such vector particles can, under certain circumstances, mediate the transfer of genes into the cells they infect. Such cells are designated herein as “target cells”. When the vector particles are used to transfer genes into cells which they infect, such vector particles are also designated “gene delivery vehicles” or “delivery vehicles”. Retroviral vectors have been used to transfer genes efficiently by exploiting the viral infectious process. Foreign genes cloned into the retroviral genome can be delivered efficiently to cells susceptible to infection or transduction by the retrovirus. Through other genetic manipulations, the replicative capacity of the retroviral genome can be destroyed. The vectors introduce new genetic material into a cell but are unable to replicate.

### II. Prostatic Acid Phosphatase (PAP)

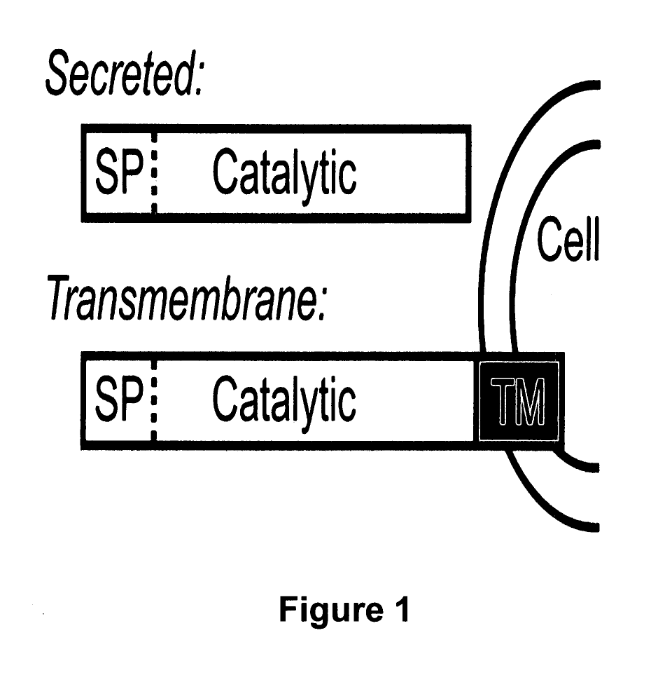

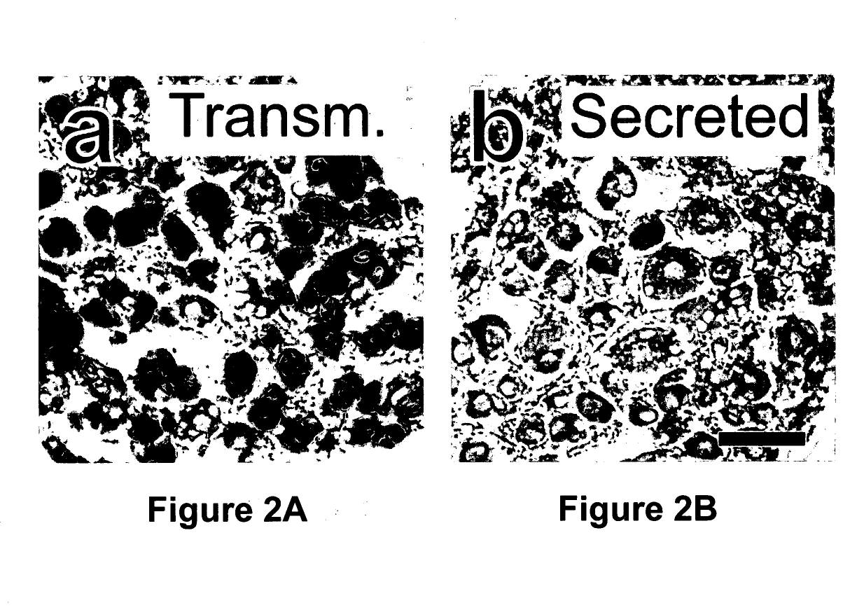

PAP is a member of the histidine acid phosphatase superfamily. Histidine acid phosphatases contain a highly conserved RHGXRXP (SEQ ID NO: 1) motif located within the active site. PAP can be made catalytically inactive, for example, by methods including heat denaturation and by incubating the protein with diethylpyrocarbonate (DEPC), which chemically modifies all histidine residues, or by mutating the active site histidine residue (His12) to alanine (McTigue and Van Etten, 1978; Ostanin et al., 1994). As its name implies, PAP is predominantly expressed in prostate, although the presently disclosed subject matter shows PAP is also expressed at high levels in small diameter DRG neurons (Examples 3-5, FIGS. 1 and 2A). PAP is expressed as either a secreted (soluble) protein or as a type 1 transmembrane (TM) protein, with the catalytic phosphatase domain located extracellularly (FIG. 1). The secreted form has been extensively studied and is used as a blood diagnostic marker for prostate cancer (Ostrowski and Kuciel, 1994; Roiko et al., 1990).

Fluoride-Resistant Acid Phosphatase (FRAP) is a classic histochemical marker of many small-diameter dorsal root ganglia (DRG) neurons and is implicated in pain mechanisms. The molecular identity of FRAP was unknown. Using genetic approaches, the presently disclosed subject matter demonstrates that a transmembrane isoform of Prostatic Acid Phosphatase (PAP, EC 3.1.3.2) is FRAP. Pain-sensing peptidergic and nonpeptidergic nociceptive neurons of mice and humans express PAP suggesting an unanticipated role for PAP in pain (Examples 3-5).

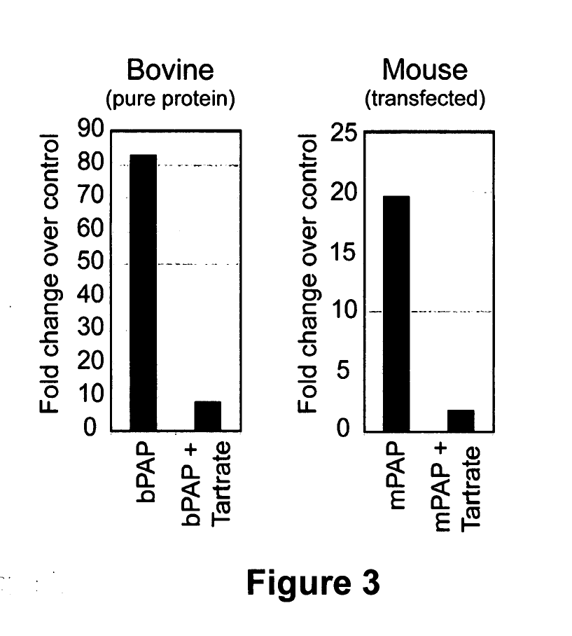

PAP and FRAP have many features in common. For example, FRAP is localized to plasma membrane, golgi and endoplasmic reticulum by electron microscopy, and is particularly enriched near the presynaptic membrane of DRG neurons (Csillik and Knyihar-Csillik, 1986; Knyihar-Csillik et al., 1986; Knyihar and Gerebtzoff, 1970). These ultrastructural data are consistent with the fact that transmembrane PAP is the predominant isoform in DRG (Examples 3-5). PAP and FRAP are also both reversibly inhibited by L-tartrate (FIG. 3; Example 6). PAP and FRAP are both down-regulated in nociceptive circuits after sciatic nerve transection (Costigan et al., 2002; Csillik and Knyihar-Csillik, 1986; Example 3; Table 2). PAP and FRAP are classified as acid phosphatases; however, they are both catalytically active at acidic (pH 5) and neutral pH. PAP and FRAP dephosphorylate the same substrates including phosphoryl-o-tyrosine, phosphoryl-o-serine, para-nitrophenyl phosphate (p-NPP), thiamine monophosphate and nucleotides (particularly nucleotide monophosphates, such as adenosine monophosphate; AMP) (Ostrowski and Kuciel, 1994; Silverman and Kruger, 1988a).

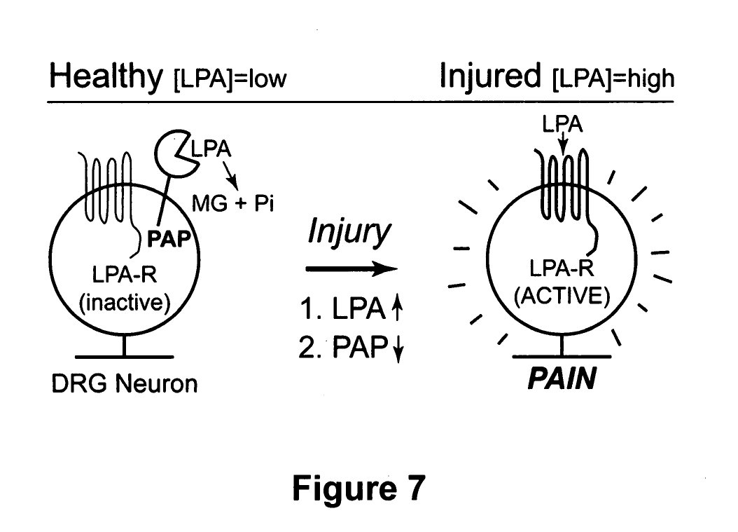

Several groups have also found that PAP dephosphorylates lysophosphatidic acid (LPA) to monoglyceride (MG) and inorganic phosphate (FIG. 7) (Hiroyama and Takenawa, 1999; Tanaka et al., 2004). In fact, increasing PAP levels by over-expression caused decreased proliferation of prostate cancer cells (Lin et al., 1994). Decreased proliferation could be attributed to the fact that PAP inactivates LPA, blocking its mitogenic effects (Tanaka et al., 2004). In support of this hypothesis, loss of PAP activity in PAP−/− mice leads to hyperproliferation of prostate cells (Vihko unpublished).

Lysophosphatidic Acid (LPA) is a potent lysophospholipid mediator that regulates many biological processes, including proliferation, differentiation, survival, and pain (Brindley et al., 2002; Inoue et al., 2004; Moolenaar, 2003; Moolenaar et al., 2004; Tigyi et al., 1994). LPA is released from platelets upon wounding as well as from neurons and other cells (Eichholtz et al., 1993; Sugiura et al., 1999; Xie et al., 2002).

There are four well-characterized LPA receptors, called LPA1, LPA2, LPA3 and LPA4 (Anliker and Chun, 2004; Noguchi et al., 2003; Takuwa et al., 2002). These receptors couple to diverse downstream signaling molecules and are expressed in many cells throughout the body. LPA1 and LPA3 are also expressed in DRG neurons (see Example 5; Inoue et al., 2004; Renback et al., 2000). In addition, Lee et al. found a fifth LPA receptor called LPA5 and demonstrated that it is also expressed in DRG (Lee et al., 2006). LPA receptor activation is routinely measured using calcium imaging, Mitogen Activated Protein Kinase (MAPK) pathway activation, Elk1 transcriptional activation, and RhoA/ROCK pathway activation (Mills and Moolenaar, 2003). LPA receptor signaling is terminated by either receptor desensitization or by dephosphorylation (degradation) of LPA. There are currently several known phosphatases that dephosphorylate LPA extracellularly: 1) PAP; 2) Lysophosphatidic Acid Phosphatase (LPAP; also known as ACP6); and 3) Lipid Phosphate Phosphatases 1 through 3 (LPP1-3), also known as Phosphatadic Acid Phosphatase type 2A-C (PPAP2A-C) (Brindley et al., 2002; Hiroyama and Takenawa, 1999; Pyne et al., 2005; Tanaka et al., 2004). Using calcium imaging as readout, over-expression of LPP1 was shown to inhibit LPA-receptor signaling via dephosphorylation of LPA (Pilquil et al., 2001; Zhao et al., 2005). PAP has not been studied using such cell-based assays.

LPA has several well-documented direct effects on DRG neurons and pain-related behaviors (Park and Vasko, 2005). Elmes and colleagues found that intracellular calcium levels were increased in small-diameter DRG neurons following stimulation with LPA (Elmes et al., 2004). LPA was also shown to increase action potential duration and frequency in wide dynamic range neurons located in the dorsal spinal cord, and to increase nociceptive flexor responses when injected into the hindpaw (Elmes et al., 2004; Renback et al., 1999). When injected into skin, LPA has been shown to cause itching/scratching behaviors (Hashimoto et al., 2006; Hashimoto et al., 2004). Itch signals are transmitted from the periphery to the CNS by small diameter DRG neurons (Han et al., 2006; Schmelz et al., 1997).

Intrathecal injection of LPA has been shown to cause profound allodynia and thermal hyperalgesia that persisted for several days in mice (intrathecal=i.t.=into spinal cord cerebrospinal fluid (“CSF”)) (Inoue et al., 2004). Additionally, Inoue and colleagues demonstrated, using pharmacological and genetic approaches, that LPA receptor signaling was required for the initiation of neuropathic pain. Inoue and colleagues found that LPA1−/− mice failed to develop allodynia and thermal hyperalgesia after nerve injury. They also found that neuropathic pain could be blocked by intrathecal injection of LPA1 antisense oligonucleotides, intrathecal injection of Botulinum toxin C3 exoenzyme (BoTXC3 inhibits RhoA, which is activated downstream of LPA1), and by systemic pharmacological inhibition of ROCK (which is downstream of RhoA) (Inoue et al., 2004). Although not conclusive, their studies suggested LPA1 receptor activation in DRG was required for these effects.

Intrathecal LPA injections have also been shown to cause demyelination in sciatic nerve and up-regulation of the α2δ1 subunit of the voltage-gated calcium channel (Caa2δ1) (Inoue et al., 2004). Caα2δ1 is up-regulated in DRG in neuropathic pain models and is the target for the drug gabapentin (Field et al., 2006; Luo et al., 2001; Maneuf et al., 2006). Gabapentin is frequently prescribed to treat neuropathic pain in humans (Baillie and Power, 2006; Dworkin et al., 2003). Taken together, these studies indicate that LPA signaling plays a direct role in the physiology of DRG neurons, sensitization of nociceptive circuits, and promotion of pathological pain states.

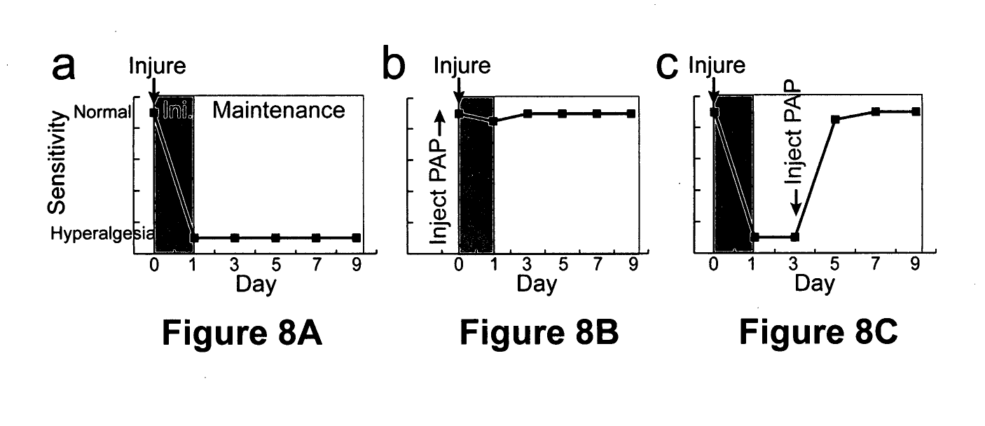

While the presently disclosed subject matter is not limited to any particular mechanism, the following is one proposed model. In healthy, uninjured animals PAP functions to dephosphorylate (degrade) LPA and maintain LPA receptors (LPA-R) in an inactive, non-signaling state (FIG. 7). Following peripheral nerve injury, LPA is released by platelets and neurons, causing extracellular LPA concentrations to abruptly rise. These abnormally high levels of LPA overwhelm the catalytic ability of transmembrane PAP to degrade LPA. These high concentrations of LPA then activate LPA receptors (FIG. 7) and initiate neuropathic pain (FIG. 7; Inoue et al., 2004). Accordingly, the initiation step can be blocked by injecting a bolus of purified, soluble PAP protein (secreted isoform) into the spinal cord cerebrospinal fluid (CSF) (FIGS. 8A-8C). This bolus of PAP will degrade excess LPA, prevent LPA receptor signaling, and thus prevent allodynia and hyperalgesia (that is, prevent initiation of neuropathic pain).

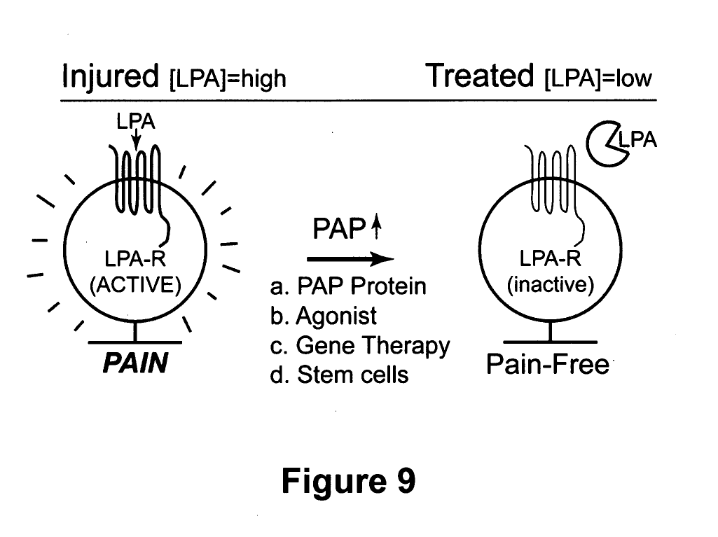

Glutamate receptor activation is also required to initiate neuropathic pain (Davar et al., 1991). LPA signaling could facilitate glutamate release by sensitizing or depolarizing neurons (Chung and Chung, 2002). After nerve injury, PAP expression and FRAP activity precipitously declines and remains low in DRG neurons (Example 3) (Costigan et al., 2002; Csillik and Knyihar-Csillik, 1986). Without PAP, LPA concentrations would be higher in injured animals compared to healthy animals. These abnormal LPA concentrations could chronically activate LPA receptors on DRG neurons. This chronic activation could sensitize DRG neurons and contribute to the allodynia and hyperalgesia that persists for days following nerve injury (during the maintenance phase) (FIG. 7). Abnormal levels of LPA could also activate microglia that are involved in the maintenance phase of neuropathic pain (Hains and Waxman, 2006; Moller et al., 2001; Schilling et al., 2004; Tsuda et al., 2003). According to the presently disclosed subject matter, PAP activity can be restored during the maintenance phase by injecting soluble PAP into spinal cord CSF (FIG. 8). Excess PAP can degrade LPA, reduce LPA-evoked signaling, and restore mechanical and thermal sensitivity to baseline values. Accordingly, in some embodiments, PAP is provided as a treatment for neuropathic pain (FIG. 9).

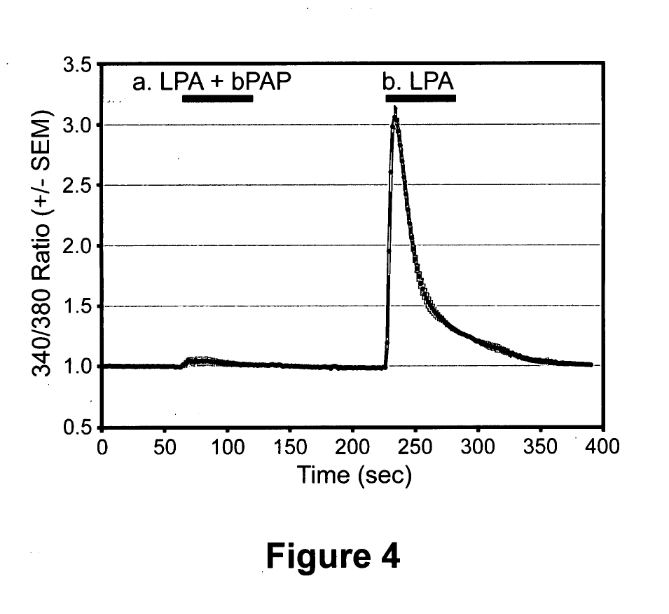

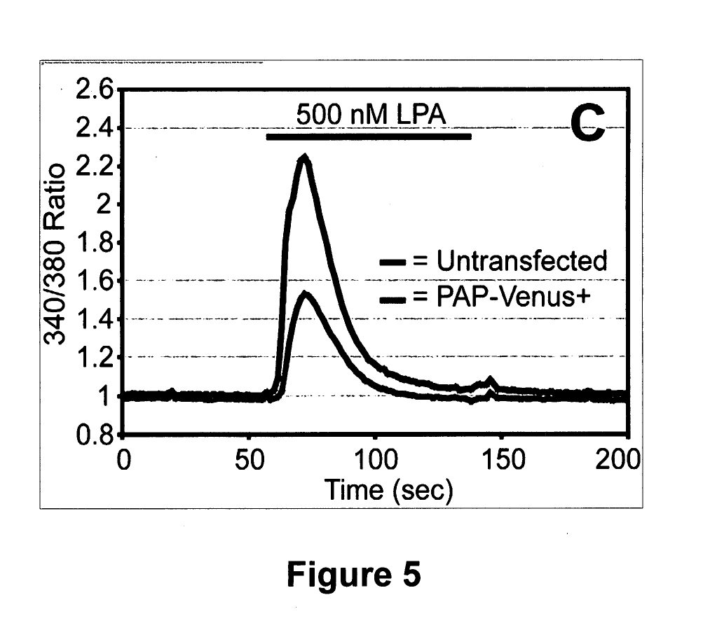

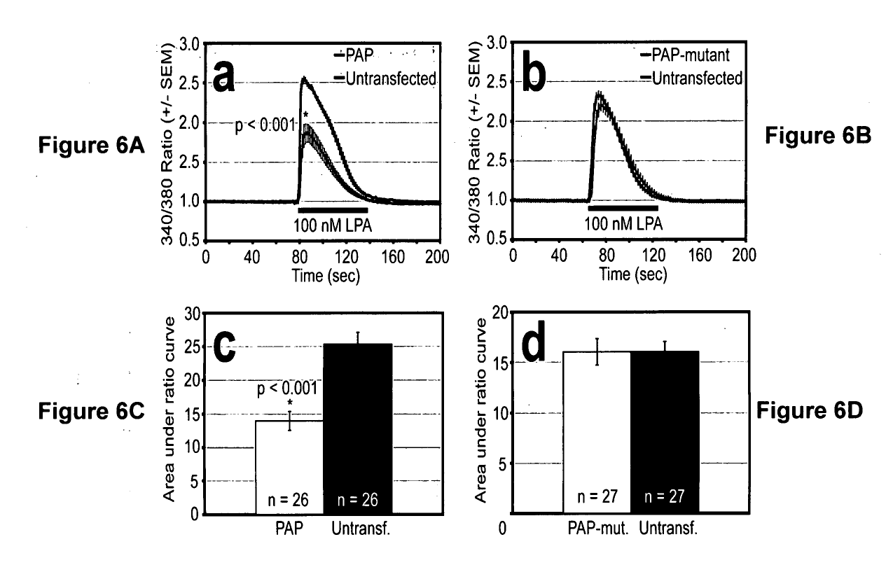

The presently disclosed subject matter demonstrates that bovine PAP inactivates LPA (Example 7; FIG. 4). As can be seen in FIG. 4, intracellular calcium levels did not appreciably change when Rat1 cells were stimulated with LPA+bPAP; however, intracellular calcium levels dramatically changed when these same cells were stimulated with LPA alone. These data clearly indicate that bPAP dephosphorylates and inactivates LPA. In addition, FIG. 5 shows that mouse PAP, via dephosphorlyation of LPA, acutely reduces LPA-evoked signaling in a cell-based context (Example 8). To further demonstrate that PAP modulation of LPA-signaling is dependent on phosphatase activity, a phosphatase-dead mouse PAP expression construct (PAP-mutant) was engineered by mutating the active site residue Histidine 12 to Alanine. Then, Rat1 fibroblasts were transfected with PAP or PAP-mutant, and calcium responses were compared in PAP transfected cells to untransfected cells in the same field of view (Example 9). As can be seen in FIGS. 6A-6D, the LPA-evoked calcium response was significantly reduced in PAP transfected cells as opposed to PAP-mutant transfected cells. These results show that the reduced LPA response in PAP transfected cells is dependent on PAP phosphatase activity. These findings suggest that PAP inactivates LPA through dephosphorylation.

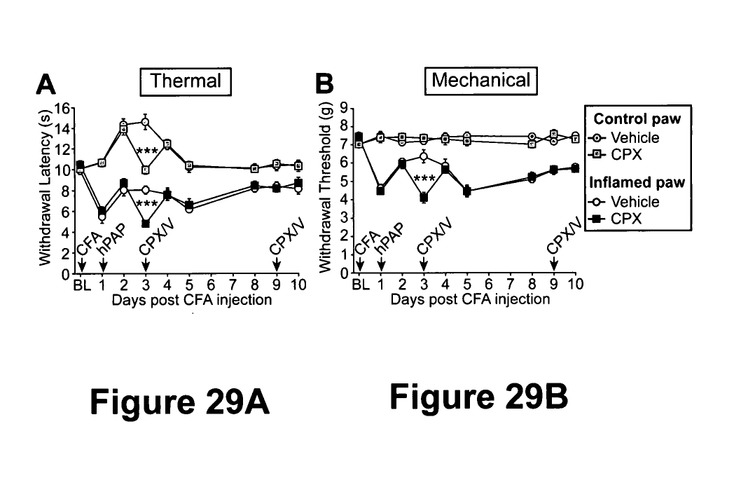

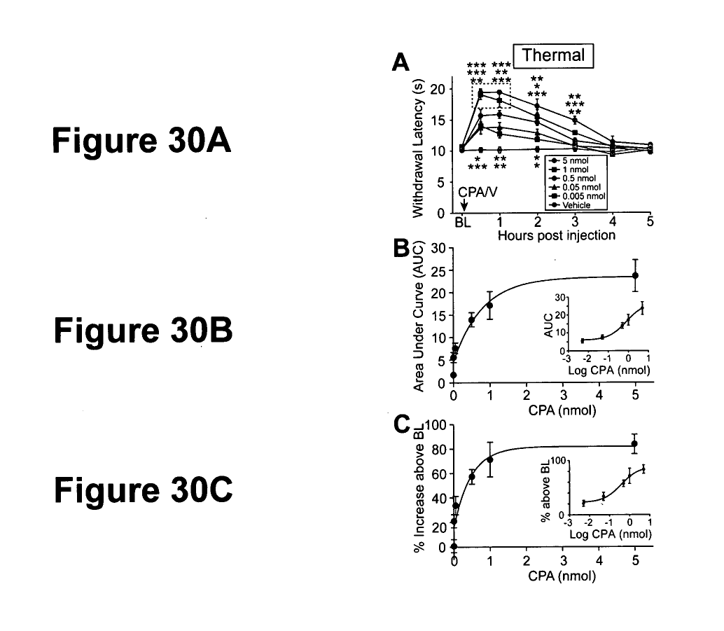

Again, without being bound to any one mechanism of action, the presently disclosed subject matter further relates to the ability of PAP to act as a ectonucleotidase and suppress pain by generating adenosine. As described in Example 13, the in vivo effects of PAP on acute and chronic pain appear to mimic the effects of i.t. adenosine and A₁-receptor (A₁R) antagonists. See FIG. 30. Further, it appears that PAP anti-nociception can be transiently inhibited with an Al R antagonist. See FIG. 29.

### III. Representative Embodiments

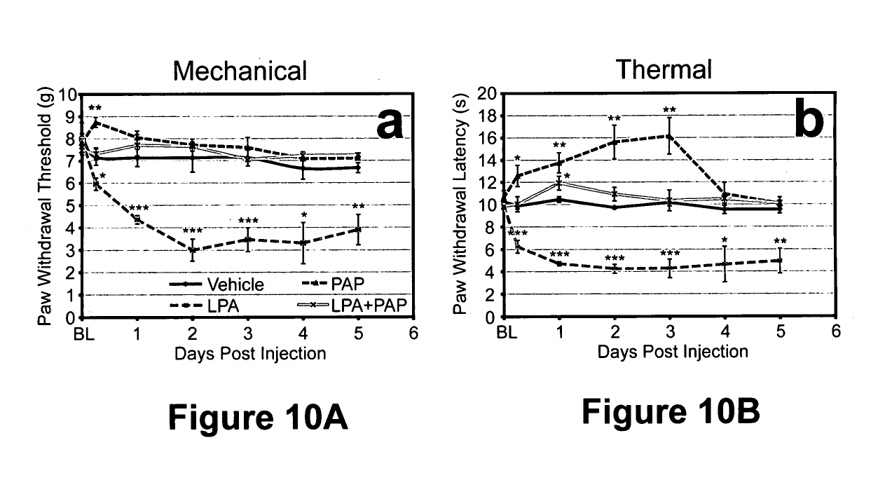

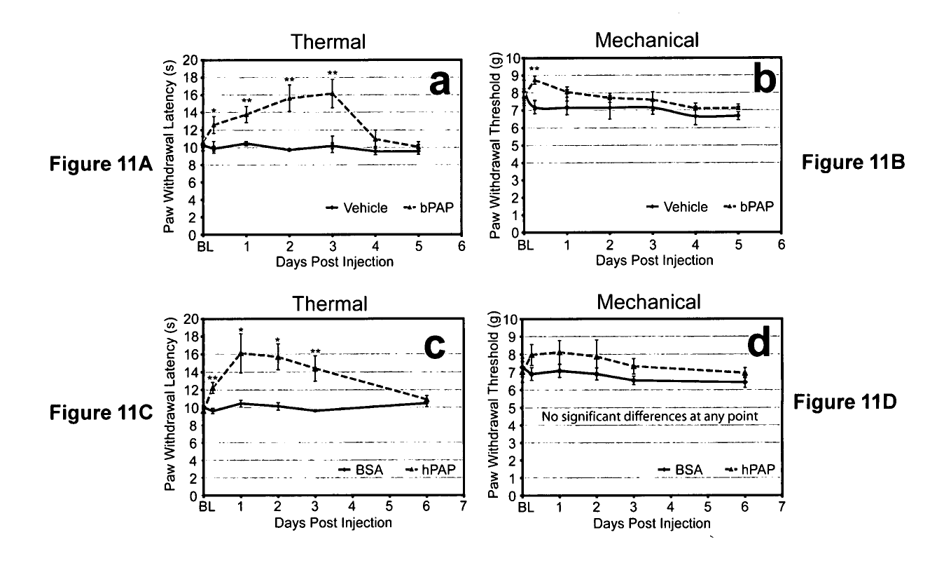

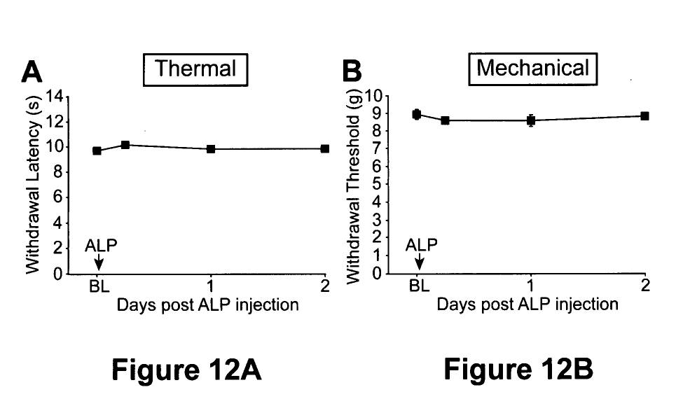

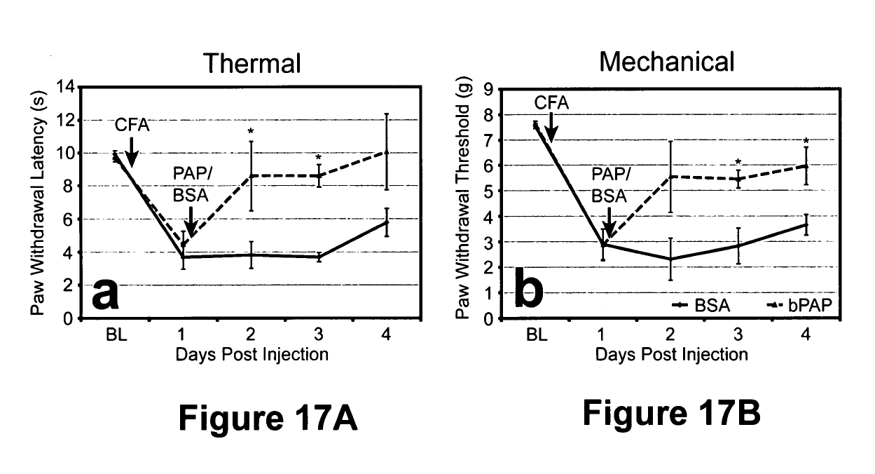

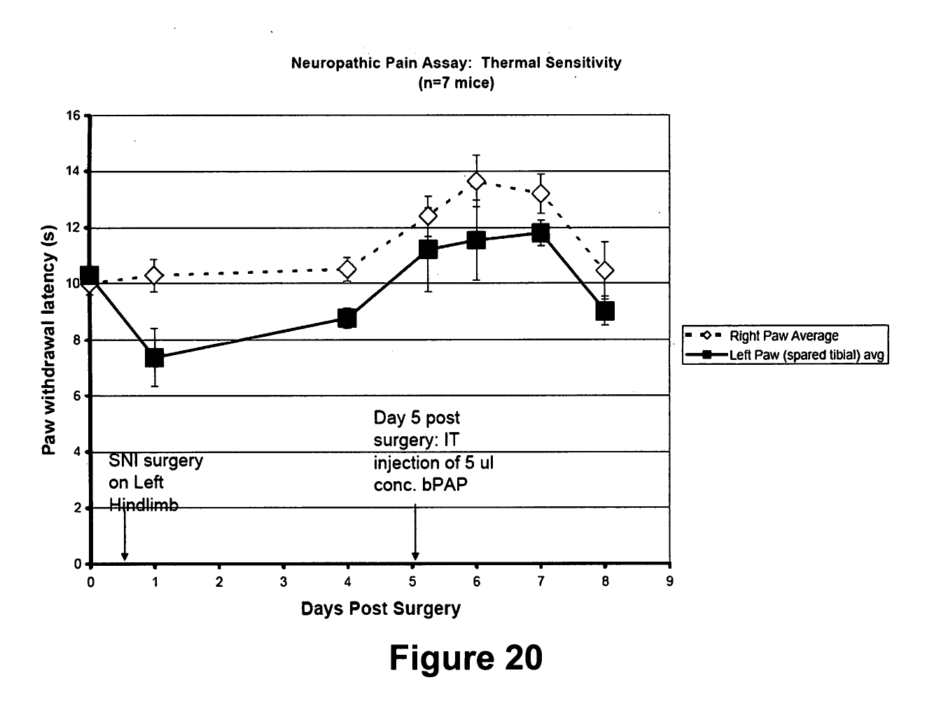

Examples 10-11 demonstrate that PAP functions as an analgesic in mice for a period of 3 days after injection into cerebrospinal fluid. FIGS. 10A and 10B show that intrathecal injection of active bovine PAP inhibits LPA-evoked mechanical and thermal sensitization in mice. FIGS. 11A-11D, 13, and 14A-14C show that intrathecal injection of active human or bovine PAP functions as an analgesic and reduces thermal sensitivity in mice, while FIGS. 12A and 12B show that another phosphatase, bovine alkaline phosphatase (ALP) does not reduce termal or mechanical sensitivity. FIGS. 17A-17B, 18, and 19 show that bovine and human PAP can reduce chronic mechanical and thermal inflammatory pain in mice. FIGS. 20-23 show that allodynia and hyperalgesia due to nerve injury can be prevented by increasing PAP activity in spinal cord. For example, spared nerve injury (SNI) surgery-induced neuropathic pain causes hyperalgesia to thermal stimuli in the injured paw. Injection of either human or bovine PAP significantly reduces hyperalgesia for about 3 days in the SNI-injured paw and produces analgesia in the uninjured paw. SNI surgery-induced mechanical sensitivity (allodynia) is also significantly reduced for about 3 days following injection of hPAP or bPAP. hPAP and bPAP do not alter mechanical sensitivity in uninjured paw. The foregoing data demonstrate that a single dose of PAP treats chronic pain to the point that mice almost fully recover. Example 12 demonstrates that PAP inhibits alloydynia and hyperanalgesia in PAP knockout mice.

Accordingly, PAP is provided as a treatment for chronic pain, including but not limited to neuropathic and inflammatory pain in animals and humans. PAP, an active variant, fragment or derivative thereof, or a small molecule modulator of PAP is provided in the presently disclosed subject matter. PAP, or an active variant, fragment or derivative thereof, can be administered by intrathecally injecting purified PAP protein or by administering (via all possible routes) small-molecule modulators to activate PAP that is normally present on pain-sensing neurons. These treatments could be used pre- or post-operatively to treat surgical pain; to treat pain associated with childbirth; to treat chronic inflammatory pain (osteoarthritis, burns, joint pain, lower back pain) to treat visceral pain, migraine headache, cluster headache, headache and fibromyalgia and to treat chronic neuropathic pain. Neuropathic pain is caused by nerve injury, including but not limited to injuries resulting from trauma, surgery, cancer, viral infections like Shingles and diabetic neuropathy.

The secreted isoform of human PAP protein is commercially available and PAP circulates in the blood of males (Vihko et al., 1978a). This suggests injection of PAP protein into patients suffering from pain will be well-tolerated. Moreover, PAP is a “druggable” protein, as selective PAP inhibitors have been previously identified by pharmaceutical companies (Beers et al., 1996). PAP activators or allosteric modulators are also provided in this disclosure as effective drugs for the treatment of pain. Methods for identifying small-molecule modulators of PAP are provided in this disclosure. Such methods include high-throughput screens (HTS) for PAP modulators using the biochemical and cell-based assays of the presently disclosed subject matter, including the assay described in Example 12. In some embodiments, large compound libraries are screened to identify drugs that activate PAP at very low doses. PAP is considered to be expressed in many fewer tissues than LPA receptors, and small molecules that increase PAP activity can be used to treat neuropathic pain and inflammatory pain and other human diseases, such as cystic fibrosis, with more specificity and fewer side effects.

While the presently disclosed subject matter is not limited to any particular mechanism, in one model PAP causes the analgesic effect disclosed herein by catalyzing the conversion of adenosine monophosphate (AMP) to adenosine. Experimental results show that PAP can dephosphorylate AMP in spinal cord tissue. In addition, adenosine is analgesic and reduces allodynia in humans suffering from neuropathic pain (Lynch et al., 2003; Sjolund et al., 2001). AMP is converted to adenosine when injected into rodent spinal cord and causes analgesia via adenosine receptor activation (Patterson et al., 2001). Thus, in some embodiments of the presently disclosed subject matter PAP is co-administered with AMP for the treatment of pain. In some embodiments, AMP analogs that can be dephosphorylated by PAP to adenosine are co-administered with PAP. In some embodiments, these analogs are more stable in biological tissues, are lipophilic, and have favorable drug metabolism and pharmacokinetics (DMPK). In some embodiments of the presently described subject matter, the administration of PAP for the treatment of pain is in combination with one or more of adenosine, adenosine monophosphate (AMP), an AMP analogue, an adenosine kinase inhibitor, adenosine kinase inhibitor 5′-amino-5′-deoxyadenosine, adenosine kinase inhibitor 5-iodotubercidin, an adenosine deaminase inhibitor, adenosine deaminase inhibitor 2′-deoxycoformycin, a nucleoside transporter inhibitor, nucleoside transporter inhibitor dipyridamole. In some embodiments of the presently described subject matter, the administration of PAP for the treatment of pain is in combination with one or more known analgesic, including, but not limited to, an opiate (e.g., morphine, codeine, etc.).

Adenosine and adenosine receptor agonists are being tested in the art as treatments for cystic fibrosis (CF). In some embodiments, PAP is aerosolized into the lungs of patients to convert endogenous AMP to adenosine and thus to serve as a treatment for CF.

There are several pain conditions that differentially affect males and females (Craft et al., 2004; Giles and Walker, 1999). PAP expression is androgen regulated in prostate (Porvari et al., 1995). In some embodiments of the presently disclosed subject matter, PAP is useful to treat and diagnose a variety of pain conditions that impact human health. In some embodiments, a method is provided for diagnosing an individual's response to a pain medicine comprising identifying one or more single nucleotide polymorphisms (SNPs), insertions or deletions in and around a PAP genomic locus in the individual; and correlating the SNPs with a predetermined response to the pain medicine. In some embodiments, a method is provided for diagnosing an individual's threshold for pain, comprising identifying one or more single nucleotide polymorphisms (SNPs), insertions or deletions in and around a PAP genomic locus in the individual; and correlating the SNPs with a predetermined threshold for pain. In some embodiments, a method is provided for correlating the differential expression of PAP in male and female DRG neurons with pain response, the method comprising: determining the extent to which a PAP is differentially expressed in male and female DRG neurons; and identifying a differential response to pain or to a pain medicine between the males and females; and correlating the extent of differential expression with the differential response to pain or to the pain medicine.

### IV. PAP-Containing Compositions

Preparations of PAP protein for use in embodiments of the presently disclosed subject matter can be prepared using a variety of methods. Human PAP is commercially available from Sigma-Aldrich and other vendors. Production of the PAP generally requires quality control to ensure the preparation is sterile, endotoxin free and acceptable for use in humans.

Recombinant methods of obtaining suitable preparations of PAP or active PAP variants, fragments or derivatives are also suitable. Using a PAP cDNA (such as the cDNAs described in Example 1), recombinant protein can be produced by one of the many known methods for recombinant protein expression (see, e.g. Vihko et al., 1993). Isolated nucleotide sequences encoding for the PAP peptide of the presently disclosed subject matter and expression vectors comprising these nucleotides are provided. Host cells comprising the expression vectors are also provided. The presently disclosed subject matter includes viral vector transfer cassettes, such as but not limited to, adenoviral, adeno-associated viral, and retroviral vector transfer cassettes comprising a nucleotide sequence encoding a PAP or active variant or fragment thereof.

Active PAP variants and fragments can be produced using mutagenesis techniques, including site-directed mutagenesis (Ostanin et al., 1994), somatic hypermutation (Wang and Tsien, 2006) and generation of deletion constructs, to evolve versions of hPAP that are more stable or have a higher k_{cat} for substrates like LPA and AMP. Active PAP variants, fragments or derivatives of the presently disclosed subject matter can comprise one or more modifications including conservative amino acid substitutions; non-natural amino acid substitutions, D- or D,L-racemic mixture isomer form amino acid substitutions, amino acid chemical substitutions, carboxy- or amino-terminus modifications and conjugation to biocompatible molecules including fatty acids and PEG.

The term “conservatively substituted variant” refers to a peptide comprising an amino acid residue sequence substantially identical to a sequence of a reference peptide in which one or more residues have been conservatively substituted with a functionally similar residue and which displays the activity as described herein for the reference peptide (e.g., of the PAP). The phrase “conservatively substituted variant” also includes peptides wherein a residue is replaced with a chemically derivatized residue, provided that the resulting peptide displays the activity of the reference peptide as disclosed herein.

Examples of conservative substitutions include the substitution of one non-polar (hydrophobic) residue such as isoleucine, valine, leucine or methionine for another; the substitution of one polar (hydrophilic) residue for another such as between arginine and lysine, between glutamine and asparagine, between glycine and serine; the substitution of one basic residue such as lysine, arginine or histidine for another; or the substitution of one acidic residue, such as aspartic acid or glutamic acid for another.

Peptides of the presently disclosed subject matter also include peptides comprising one or more additions and/or deletions or residues relative to the sequence of a peptide whose sequence is disclosed herein, so long as the requisite activity of the peptide is maintained. The term “fragment” refers to a peptide comprising an amino acid residue sequence shorter than that of a peptide disclosed herein.

PAP, and particularly a smaller molecular weight active PAP variant, fragment or derivative, can be obtained by chemical synthesis using conventional methods. For example, solid-phase synthesis techniques can be used to obtain PAP or an active variant, fragment or derivative thereof.

In some embodiments, PAP preparations are provided where PAP protein or an active PAP variant, fragment or derivative is complexed to an immobile support including supports such as agarose, sepharose, and nanoparticles. Through such immobilization, PAP is protected from degradation and remains in situ for longer periods of time. In this manner, the three day window of PAP analgesia observed herein in some embodiments can be extended to weeks or months.

### V. Methods of Treatment

PAP can be administered by a variety of methods for the treatment of pain and cystic fibrosis in animals. The PAP, the active variant, fragment or derivative thereof, and/or the PAP modulator can be administered via one or more of injection, oral administration, suppository, a surgically implanted pump, aerosolizing into the lungs, stem cells, viral gene therapy, or naked DNA gene therapy. Injection can include any type of injection, such as, but not limited to, intravenous injection, epideral injection or intrathecal injection.

In some embodiments, a small molecule modulator of PAP activity is administered by oral administration.

In some embodiments, a therapeutically effective amount of a composition or pharmaceutical formulation comprising a PAP, or an active variant, fragment or derivative thereof, is administered to the animal or human by injection. Any suitable method of injection, such as intrathecal, intravenous, intraarterial, intramuscular, intraperitoneal, intraportal, intradermal, epideral, or subcutaneous can be used. In some embodiments, PAP is dispersed in any physiologically acceptable carrier that does not cause an undesirable physiological effect. Examples of suitable carriers include physiological saline and phosphate-buffered saline. The injectable solution can be prepared by dissolving or dispersing a suitable preparation of the active PAP in the carrier using conventional methods. In some embodiments, PAP is provided in a 0.9% physiological salt solution. In some embodiments, PAP is provided enclosed in liposomes such as immunoliposomes, or other delivery systems or formulations that are known in the art.

In some embodiments, a composition or pharmaceutical formulation comprising a therapeutically effective amount of a PAP, or an active variant, fragment or derivative thereof, is provided through a surgically implantable pump apparatus for delivery of PAP to local tissue. In some embodiments, the surgically implantable pump apparatus is an intrathecal drug delivery system comprising an implantable infusion pump and an implantable intraspinal catheter. See, for example, the commercially available apparatus used to deliver opiates for chronic pain treatment (Medtronic, Minneapolis, Minn., United States of America). In some embodiments, a kit is provided for the treatment of pain in animals, comprising a composition or pharmaceutical formulation comprising a therapeutically effective amount of a PAP, or an active variant, fragment or derivative thereof, and a surgically implantable pump apparatus for delivery of PAP to local tissue.

In some embodiments, an animal is treated with PAP for cystic fibrosis. In some embodiments, the animal is administered a composition or pharmaceutical formulation comprising a therapeutically effective amount of a PAP, or an active variant, fragment or derivative thereof, or a therapeutically effective amount of an activity enhancing modulator of a PAP wherein the PAP composition is aerosolized in the lungs.

In some embodiments, an animal is administered a PAP, or an active variant or fragment thereof, through intrathecal injection of embryonic stem (ES) cells expressing PAP (see, e.g., Wu et al., 2006). This method employs derivation of patient-specific ES cells by somatic cell nuclear transfer (SCNT). The feasibility of this approach has been demonstrated in animal models. Cells are produced that can be differentiated into hematopoietic stem cells (HSCs), neurons or other cell types in vitro and transplanted into a subject animal or human.

In some embodiments, the therapeutically effective amount of PAP, or an active variant, fragment or derivative thereof, can be administered once daily. In some embodiments, the dose is administered twice or three times weekly. In some embodiments, administration is performed once a week or biweekly.

In some embodiments, the therapeutically effective amount of a PAP or active variant or fragment thereof is administered by methods known to those of skill in the art as “gene therapy”. Gene therapy as used herein refers to a general method for treating a pathologic condition in a subject by inserting an exogenous nucleic acid into an appropriate cell(s) within the subject. The nucleic acid is inserted into the cell in such a way as to maintain its functionality, for example, so as to maintain the ability to express a particular polypeptide. In some embodiments, a therapeutically effective amount of a PAP is administered via viral gene therapy using a viral vector transfer cassette (e.g., a retroviral, adenoviral or adeno-associated viral cassette) comprising a nucleic acid sequence encoding the PAP or active variant or fragment thereof.

With respect to the methods of the presently disclosed subject matter, a preferred subject is a vertebrate subject. A preferred vertebrate is warm-blooded; a preferred warm-blooded vertebrate is a mammal. The subject treated by the presently disclosed methods is desirably a human, although it is to be understood that the principles of the presently disclosed subject matter indicate effectiveness with respect to all vertebrate species which are included in the term “subject.” In this context, a vertebrate is understood to be any vertebrate species in which treatment of a disorder is desirable. As used herein “subject” includes both human and animal subjects. Thus, veterinary therapeutic uses are provided in accordance with the presently disclosed subject matter.

As such, the presently disclosed subject matter provides for the treatment of mammals such as humans, as well as those mammals of importance due to being endangered, such as Siberian tigers; of economic importance, such as animals raised on farms for consumption by humans; and/or animals of social importance to humans, such as animals kept as pets or in zoos. Examples of such animals include but are not limited to: carnivores such as cats and dogs; swine, including pigs, hogs, and wild boars; ruminants and/or ungulates such as cattle, oxen, sheep, giraffes, deer, goats, bison, and camels; and horses. Also provided is the treatment of birds, including the treatment of those kinds of birds that are endangered and/or kept in zoos or as pets (e.g., parrots), as well as fowl, and more particularly domesticated fowl, i.e., poultry, such as turkeys, chickens, ducks, geese, guinea fowl, and the like, as they are also of economical importance to humans. Thus, also provided is the treatment of livestock, including, but not limited to, domesticated swine, ruminants, ungulates, horses (including race horses), poultry, and the like.

### VI. Diagnostics

In some embodiments, a subject's genotype can be used to determine valuable information for predicting the subject's response to pain and/or to pain medication. As used herein, the term “genotype” means the genetic makeup of an organism. Expression of a genotype can give rise to an organism's phenotype, i.e. an organism's physical traits. The term “phenotype” refers to any observable property of an organism, produced by the interaction of the genotype of the organism and the environment. A phenotype can encompass variable expressivity and penetrance of the phenotype. Exemplary phenotypes include but are not limited to a visible phenotype, a physiological phenotype, a susceptibility phenotype, a cellular phenotype, a molecular phenotype, and combinations thereof. The phenotype can be related to pain response and/or a response to pain medication. A particular subject's genotype can be compared to a reference genotype or the genotype of one or more other subjects to provide valuable information related to current or predictive phenotypes.

“Determining the genotype” of a subject, as used herein, can refer to determining at least a portion of the genetic makeup of an organism and particularly can refer to determining a genetic variability in a subject that can be used as an indicator or predictor of phenotype. The genotype determined can be the entire genome of a subject, but far less sequence is usually required. In some embodiments, determining the genotype comprises identifying one or more polymorphisms, including single nucleotide polymorphisms (SNPs), insertions, deletions and/or other types of genetic mutations in and around a PAP genomic locus in the subject. As used herein, the term “polymorphism” refers to the occurrence of two or more genetically determined alternative variant sequences (i.e., alleles) in a population. A polymorphic marker is the locus at which divergence occurs. Exemplary markers have at least two alleles, each occurring at a frequency of greater than 1%. A polymorphic locus may be as small as one base pair (e.g., a single nucleotide polymorphism (SNP)).

In some embodiments, the presently disclosed subject matter provides a method for diagnosing an individual's response to a pain medicine, comprising identifying one or more SNPs, insertions, deletions and/or other types of genetic mutations in and around a PAP genomic locus in the individual; and correlating the SNPs, insertions, deletions and/or other types of genetic mutations with a predetermined response to the pain medicine. For example, an individual's (or a population subset's) response to a pain medicine can be compared to the response to the pain medicine in a control population. Then, it can be determined if the individual (or population subset) has one or more genetic variations related to the PAP gene. In some embodiments, certain genetic variations can be correlated to an ability to respond to pain or to a pain medication. For example, genetic variations can be statistically correlated to particular pain response behaviours. Thus, in some embodiments, the presently disclosed subject matter provides a method for diagnosing an individual's (or a population subset's) threshold for pain and/or propensity to transition from acute to chronic pain, comprising identifying one or more single nucleotide polymorphisms (SNPs) insertions, deletions and/or other types of genetic mutations in and around a PAP genomic locus in the individual; and correlating the SNPs, insertions, deletions and/or other types of genetic mutations with a predetermined threshold for pain or propensity to transition from acute to chronic pain. In some embodiments, the method involves correlating differences in PAP expression in male and female DRG neurons, identifying a differential response to pain or to pain medicine between males and females, and correlating the extent of differential expression with the differential response to pain or to pain medicine.

Various methods of determining genetic variations such as SNP's are known in the art. For example, U.S. Pat. No. 6,972,174, provides a method of determining SNP's based on polymerase chain extension reactions adjacent to potential SNP sites. U.S. Pat. No. 6,110,709 describes a method for detecting the presence or absence of an SNP in a nucleic acid molecule by first amplifying the nucleic acid of interest, followed by restriction analysis and immobilizing the amplified product to a binding element on a solid support. PCT International Patent Publication WO9302212 describes another method for amplification and sequencing of nucleic acid in which dideoxy nucleotides are used to create amplified products of varying lengths. The varying length products are then separated and visualized by gel electrophoresis. PCT International Patent Publication WO0020853 further describes a method of detecting single base changes using tightly controlled gel electrophoretic conditions to scan for conformational changes in the nucleic acid caused by sequence changes.

### VII. Examples

The presently disclosed subject matter will now be described more fully hereinafter with reference to the accompanying Examples, in which representative embodiments are shown. The presently disclosed subject matter can, however, be embodied in different forms and should not be construed as limited to the embodiments set forth herein. Rather, these embodiments are provided so that this disclosure will be thorough and complete, and will fully convey the scope of the embodiments to those skilled in the art.

### Example 1

### Methods

Molecular biology. The full-length expression construct of ACPP-transmembrane isoform (mouse PAP) (nt 64-1317 from GENBANK® accession #NM_{—}207668; SEQ ID NO: 2) was generated by RT-PCR amplification, using C57BL/6 mouse trigeminal cDNA as template and Phusion polymerase (New England BioLabs, Beverly, Mass., United States of America). PCR products were cloned into pcDNA3.1 (Invitrogen, Carlsbad, Calif., United States of America) and completely sequenced. Isoform-specific in situ hybridization probes of ACPP, secreted variant (nt 1544-2625 from GENBANK® accession #NM_{—}019807; SEQ ID NO: 3) and ACPP, transmembrane variant (nt 1497-2577 from GENBANK® accession #NM_{—}207668; SEQ ID NO: 4) were generated by PCR amplification, using C57BL/6 mouse genomic DNA as template and Phusion polymerase. Probes were cloned into pBluescript-KS (Stratagene, La Jolla, Calif., United States of America) and completely sequenced.

A pFastBAC baculovirus expression vector was generated that contains the secreted isoform of mouse PAP (nt 64-1206 from GENBANK® accession #NM_{—}019807; SEQ ID NO: 5) fused to a carboxyl-terminal thrombin cleavage site-hexahistidine tag. Similarly, a pFastBAC baculovirus expression vector was generated that contains the secreted isoform of human PAP (nt 43-1200 from GENBANK® accession #NM_{—}001099; SEQ ID NO: 6) fused to a carboxyl-terminal thrombin cleavage site-hexahistidine tag.

In situ Hybridization. In situ hybridization was performed as described in Dong et al. using digoxygenin-labeled antisense and sense (control) riboprobes.

Cell Culture. HEK 293 cells were grown at 37° C., 5% CO₂, in Dulbecco's Modified Eagle's Medium (DMEM), high glucose, supplemented with 1% penicillin, 1% streptomycin and 10% fetal bovine serum. For transfections, 6×10⁵ cells were seeded per well in 6-well dishes. Cells were cotransfected with 0.5 μg ACPP-transmembrane isoform and 0.5 μg farnesylated EGFP (EGFPf) using Lipofectamine Plus (Invitrogen, Carlsbad, Calif., United States of America). Twenty-four hours post transfection, samples were imaged for intrinsic EGFPf fluorescence to confirm that all cells were transfected. Cells were then fixed with 4% paraformaldehyde in phosphate buffered saline (PBS) and stained using FRAP histochemistry.

Tissue Preparation. All procedures involving vertebrate animals were approved by Institutional Animal Care and Use Committees at the University of North Carolina at Chapel Hill and at the University of Oulu.

For FRAP histochemistry, wild-type and PAP−/− adult male mice, ages 6-12 weeks, were anesthetized with pentobarbital and perfused transcardially with 20 mL 0.9% saline (4° C.) followed by 25 mL fixative (4% paraformaldehyde, 0.1 M phosphate buffer, pH 7.3 at 4° C.). The spinal column was dissected then cryoprotected in 20% sucrose, 0.1 M phosphate buffer, pH 7.3 at 4° C. (for 2-3 days). Spinal cord encompassing the lumbar enlargement (L4-L6 region) and L4-L6 DRG were carefully dissected and frozen in OCT.

For immunofluorescence staining, wild-type adult male mice were sacrificed by cervical dislocation or decapitation. Lumbar spinal cord and DRG (L4-L6) were dissected then postfixed for 6 hr and 2 hr, respectively. Tissues were cryoprotected in 20% sucrose, 0.1 M phosphate buffer, pH 7.3 at 4° C. for 24 hours, frozen in OCT, sectioned with a cryostat at 15-20 μm, and mounted on Superfrost Plus slides. Slides were stored at −20° C. Free-floating sections were sectioned at 30 μm and immediately stained.

FRAP Histochemistry. FRAP/Thiamine Monophosphatase (TMPase) histochemistry was performed essentially as described by Shields et al., 2003, with modifications suggested by Silverman and Kruger, 1988. Cells or tissue sections were washed twice with 40 mM Trizma-Maleate (TM) buffer, pH 5.6., then once with TM buffer containing 8% (w/v) sucrose. To precipitate lead on cells and axons bearing FRAP, samples were incubated at 37° C. for 2 hr in TM buffer containing 8% sucrose (w/v), 6 mM thiamine monophosphate chloride, 2.4 mM lead nitrate. Lead nitrate must be made fresh immediately prior to use. To reduce nonspecific background staining, samples were washed once with 2% acetic acid for one minute. Samples were then washed three times with TM buffer, developed for 10 seconds with 1% sodium sulfide, washed several times with PBS, pH 7.4, and mounted in Gel/Mount (Biomeda Corp., Foster City, Calif., United States of America). Images were acquired using a Zeiss Axioskop and Olympus DP-71 camera.

When assaying HEK 293 cells for FRAP activity, duplicate samples were stained with and without 0.1% Triton X-100 in the initial TM wash. FRAP histochemical staining was stronger in detergent permeabilized cells, presumably detecting intracellular stores of TM-PAP in the endoplasmic reticulum and golgi apparatus.

Immunofluorescence. Free-floating and slide-mounted sections were washed 3 times with 50 mM Tris base, 460 mM NaCl, 0.3% Triton X-100, pH 7.6 (TBS+TX; the high-salt concentration was essential for optimal PAP antibody staining), blocked for 60 minutes in TBS+TX4 containing 10% goat serum, then incubated overnight at 4° C. with primary antibodies diluted in blocking solution The antibodies used included: 1:1000 rabbit anti-GFP (A-11122, Molecular Probes, Eugene, Oreg., United States of America), 1:1000 chicken anti-GFP (GFP-1020, Ayes Labs, Tigard, Oreg., United States of America), 1:250 mouse anti-NeuN (MAB377, Chemicon, Billerica, Mass., United States of America), 1:800 guinea pig anti-CGRP (T-5027, Peninsula Laboratories, Inc., San Carlos, Calif., United States of America), 1:750 rabbit anti-CGRP (T-4032, Peninsula Laboratories, Inc., San Carlos, Calif., United States of America), 1:1000 rabbit anti-P2X3 (AB5895, Chemicon, Billerica, Mass., United States of America), 1:300 guinea pig anti-P2X3 (GP10108, Neuromics, Edina, Minn., United States of America), 1:100 mouse anti-PKCγ (clone PKC66, Cat. #13-3800, Zymed Laboratories, Inc., South San Francisco, Calif., United States of America), 1:1000 rabbit anti-PKCγ (c-19, Cat. #sc-211, Santa Cruz Biotechnology, Inc., Santa Cruz, Calif., United States of America), 1:1000 rabbit anti-human PAP (Biomeda Corporation, Foster City, Calif., United States of America).

Biomeda Anti-PAP antibody specificity was confirmed by: a) absence of staining when primary antibody was excluded, and b) absence of staining in DRG and spinal cord sections from PAP−/− mice. Mrgprd-expressing cells and axons were visualized by staining tissue from MrgprdΔ ^{EGFPf} mice with anti-GFP antibodies. Sections were then washed three times with TBS+TX and incubated for 2 hours at room temperature with secondary antibodies. All secondary antibodies were diluted 1:250 in blocking solution, and were conjugated to Alexa-488, Alexa-568, or Alexa-633 fluorochromes (Molecular Probes, Eugene, Oreg., United States of America), or to FITC, Cy3, or Cy5 fluorochromes (Jackson ImmunoResearch, West Grove, Pa., United States of America). To detect IB4-binding, 1:100 Griffonia simplicifolia isolectin GS-IB4-Alexa 488 (1-21411, Molecular Probes, Eugene, Oreg., United States of America) was included during secondary antibody incubations. It was necessary to amplify the anti-PAP antibody signal by using secondary antibodies conjugated to biotin, then using either 1:250 Streptavidin-Cy3 (Jackson ImmunoResearch, West Grove, Pa., United States of America); or the Tyramide Signal Amplification kit (New England Nuclear, Boston, Mass., United States of America, following manufacturers protocol).

Following staining, sections were washed three times with TBS+TX, followed by three PBS washes and wet-mounted in Gel/Mount (Biomeda Corporation, Foster City, Calif., United States of America). Images were obtained using a Leica TCS-NT confocal microscope (Leica Microsystems, Wetzlar, Germany). All cell counts are represented as percentages +/−Standard Error of the Mean (SEM).

Immunofluorescence combined with FRAP histochemistry. To demonstrate overlap between immunofluorescence and FRAP histochemistry in DRG, adjacent 15 μm sections were stained with anti-PAP antibodies, and for FRAP histochemistry. Similar methods have been used previously to co-localize FRAP with antibody and lectin markers and, due to the use of adjacent sections, underestimates the number of coexpressing cells (Dodd et al., 1983; Nagy and Hunt, 1982; Silverman and Kruger, 1988; Silverman and Kruger, 1990). Technical limitations prevented sequential processing of the same DRG section for immunofluorescence and FRAP histochemistry.

To demonstrate overlap in spinal cord tissue, identical sections were first stained with anti-PAP antibodies, imaged using confocal microscopy, and then the same sections were stained histochemically for FRAP and imaged by transmitted light microscopy. This procedure is based on a published method (Wang et al., 1994). Fluorescence and transmitted light images were overlaid in Photoshop by scaling and rotating the images as necessary.

Behavior. C57BL/6 male mice, 2-3 months old, were purchased from Jackson Laboratories (Bar Harbor, Maine, United States of America) for all behavioral experiments involving PAP protein injections. All mice were acclimated to the testing room, equipment and experimenter for one day before behavioral testing. The experimenter was blind to genotype and drug treatment during behavioral testing.

Thermal sensitivity was measured by heating one hindpaw with a Plantar Test apparatus (IITC) following the Hargreaves method (Hargreaves et al., 1988). The radiant heat source intensity was calibrated so that a paw withdrawal reflex was evoked in ˜10 seconds, on average, in wild-type C57BL/6 mice. Cutoff time was 20 s. One measurement was taken from each paw per day to determine paw withdrawal latency. To perform the tail immersion assay, mice were gently restrained in a towel and the distal one-third of the tail was immersed in 46.5° C. water. Latency to withdrawal the tail was measured once per mouse. Mechanical sensitivity was measured using semi-rigid tips attached to an Electronic von Frey apparatus (IITC) as described elsewhere (Cunha et al., 2004; Inoue et al., 2004). Three measurements were taken from each paw (separated at 5 min. intervals) then averaged to determine paw withdrawal threshold in grams.

To induce persistent inflammatory pain, 20 μL Complete Freunds Adjuvant (CFA, Sigma) was injected into one hindpaw, centrally beneath glabrous skin, with a 27G needle. The spared nerve injury (SNI) model of neuropathic pain was performed as described (Shields et al., 2003).

Intrathecal injections. hPAP, bPAP and vehicle controls were injected into the lumbar region of unanesthetized mice as described (Fairbanks, 2003).

### Example 2

### Preparation of Recombinant PAP

A mouse PAP (secreted isoform; nt 64-1206 from GENBANK® accession #NM_{—}019807; SEQ ID NO: 5) baculovirus expression construct was made containing a thrombin cleavage site and hexahistidine purification tag at the C-terminus using the clone described in Example 1 and standard procedures in the art. The recombinant mouse PAP was purified using a fee-for-service Protein Purification core facility. A hPAP (secreted isoform; nt 43-1200 from GENBANK® accession #NM_{—}001099; SEQ ID NO: 6) expression construct was similarly constructed having a thrombin-hexahistidine C-terminal tag. Large quantities of recombinant hPAP protein could be produced with this construct using procedures that are known to the art. Recombinant hPAP protein is useful as a drug in human clinical trials and can be used to assess safety of intrathecal hPAP in humans.

### Example 3

### Molecular Identification of FRAP as PAP

For nearly fifty years, it has been known that many small-diameter DRG neurons contain an acid phosphatase, commonly referred to as FRAP or Thiamine Monophosphatase (Csillik and Knyihar-Csillik, 1986; Knyihar-Csillik, 1986; Colmant, 1959). FRAP was used to mark nonpeptidergic DRG neurons and their unmyelinated axon terminals in lamina II of spinal cord, as well as a subset of peptidergic (CGRP+, Substance P+) neurons (Hunt and Mantyh, 2001; Carr et al., 1990). Use of FRAP as a marker waned when it was found that certain lectins, like Griffonia simplicifolia Isolectin B4 (IB4), also marked nonpeptidergic neurons and co-localized with FRAP (Silverman and Kruger, 1988). Moreover, the gene encoding FRAP was never unequivocally identified.

In the early 1980s, Dodd and co-workers partially purified FRAP protein from rat DRG using chromatography (Dodd et al., 1983). The partially purified FRAP protein was similar in molecular weight to human prostatic acid phosphatase (PAP) and was inhibited by L(+)-tartrate, a non-selective inhibitor of several acid phosphatases. These biochemical experiments hinted that FRAP might be PAP. However, antibodies raised against the partially purified FRAP protein and antibodies against human PAP did not immunostain small-diameter DRG neurons and their axon terminals in lamina II of the spinal cord (Silverman and Kruger, 1988, Dodd et al., 1983). These inconclusive immunohistochemical findings cast doubt as to whether or not PAP was identical to FRAP.

To resolve this ambiguity, the relationship between FRAP and PAP was re-examined using molecular, genetic and immunohistochemical techniques. PAP is expressed as either a secreted protein or as a type 1 transmembrane (TM) protein, with the catalytic acid phosphatase domain localized extracellularly (Kaija et al., 2006; Roiko et al., 1990). See FIG. 1. The secreted form has been studied extensively and is functionally linked to prostate cancer (Kaija et al., 2006). The transmembrane variant contains a single hydrophobic domain near the carboxyl (Hunt and Mantyh, 2001) terminus based on hydrophobicity analysis.

To determine if either PAP isoform was expressed in small-diameter DRG neurons like FRAP, in situ hybridization was performed with isoform-specific probes. These studies revealed that TM-PAP was expressed in a subset of small-diameter DRG neurons (see FIGS. 1 and 2A), while the secreted isoform was expressed at low to undetectable levels. See FIG. 2B.

Next, the extent to which FRAP histochemical activity was dependent on PAP enzymatic activity was directly tested. To do this, mouse TM-PAP was over-expressed in HEK 293 cells, and the cells were stained using FRAP histochemistry. While control cells transfected with empty vector did not show signs of staining, cells transfected with TM-PAP were heavily stained when the plasma membrane was left intact or was permeabilized with detergent. This indicated that TM-PAP was sufficient for FRAP histochemical activity and that TM-PAP could dephosphorylate substrates extracellularly. Similar results were obtained when TM-PAP was transfected into Rat1 fibroblasts.

DRG and spinal cord tissues from PAPΔ3/Δ3 (henceforth referred to as PAP−/−) knock-out mice were also analyzed. In these mice, deletion of exon 3 causes PAP protein truncation and complete loss of PAP catalytic activity in prostate. Strikingly, FRAP histochemical staining of DRG neurons and axon terminals in spinal cord were abolished in PAP−/− mice.

Absence of FRAP staining was not due to developmental loss of neurons or axon terminals in PAP−/− mice. Wild-type and PAP−/− mice had equivalent numbers of P2X3+ neurons relative to all NeuN+ neurons in lumbar ganglia (43.4+/−1.9% verses 42.4+/−1.9% 5 (s.e.m.); not significantly different, paired t-test; n=1500 NeuN+ neurons counted per genotype). P2X3 marks nonpeptidergic DRG neurons and is extensively co-localized with PAP. Moreover, confocal image analysis revealed no gross anatomical differences between genotypes (n=2 mice from each genotype) when spinal cord was examined using antibodies to CGRP (to mark peptidergic nerve endings), isolectin B4 (IB4, to mark non-peptidergic nerve endings) and antibodies to protein kinase C-γ (PKCγ, to mark interneurons in laminas Ilinner and III).

These data indicate that PAP is the only acid phosphatase in DRG and spinal cord with FRAP-like activity. Moreover, these gain- and loss-of function experiments conclusively demonstrate that FRAP in small-diameter DRG neurons is encoded by PAP.

Experiments were performed to show that PAP is similarly expressed in human DRG tissue. FRAP histochemical activity is located in small diameter DRG neurons in humans (Silverman and Kruger, 1988a). RT-PCR was performed using total RNA from human DRG (Clontech, Palo Alto, Calif., United States of America) as a template, and intron-spanning primers to human PAP (intron-spanning primers ensure that the amplification product originates from cDNA, not genomic DNA). A band of the correct size was obtained after only 30 cycles. This finding, combined with published FRAP histochemical data, strongly suggest human small-diameter (presumably nociceptive) neurons express PAP.

PAP protein and FRAP histochemical activity were also found to co-localize at the cellular level in DRG neurons. To do this, several commercially available anti-human hPAP antisera were purchased and tested on mouse prostate (positive control), DRG and spinal cord tissues (no commercially available anti-mouse or anti-rat PAP antibodies exist). One rabbit polyclonal antiserum stained prostate epithelial cells, small-diameter DRG neurons and axon terminals within lamina II of the spinal cord; precisely where FRAP histochemistry was observed. Small diameter trigeminal ganglia neurons and axons in lamina II of nucleus caudalis were also labeled by the antibody. Trigeminal neuron staining suggests PAP could be effective at treating pain associated with the head, such as headache or dental pain. Antibody specificity was confirmed by: a) absence of staining when primary antibody was excluded, and b) absence of staining in DRG and spinal cord sections from PAP−/− mice.

Expression of TM-PAP suggested that PAP protein is localized extracellularly, on the plasma membrane of DRG neurons (Quintero et al., 2007). This was confirmed by surface labeling of live, dissociated mouse DRG neurons using the anti-PAP antibody.

DRG neurons and spinal cord were double-labeled with antibodies to determine if PAP was expressed in peptidergic or nonpeptidergic nociceptive circuits (Table 2). Mouse L4-L6 DRG neurons and lumbar spinal cord sections were double-labeled with antibodies against various sensory neuron markers and with antibodies against PAP. Tissue from adult MrgprdΔ^{EGFPf} mice was used to identify Mrgprd-expressing neurons (Zylka et al., 2005). IB4 and MrgprdΔ^{EGFPf} are markers of nonpeptidergic neurons and endings while CGRP is a marker of peptidergic neurons and endings. These studies revealed that PAP protein was primarily localized to nonpeptidergic neurons and their axon terminals in lamina II of the mouse spinal cord.

Table 2 shows the results of quantitative analysis of PAP and sensory neuron marker colocalization studies within mouse L4-L6 DRG neurons. Images were acquired by confocal microscopy. At least 350 cells were counted per combination. Cell counts from confocal images revealed that virtually all nonpeptidergic DRG neurons co-expressed PAP: 91.6% of all IB4+ (n=497 cells counted), 99.2% of all Mrgprd+ (n=357 cells counted), and 92.6% of all P2X3+ neurons (n=824 cells counted) expressed PAP (Zylka et al., 2005). A smaller percentage (17.1%) of peptidergic CGRP+ neurons (n=1364 cells counted) expressed PAP. This preferential expression of PAP in nonpeptidergic neurons is consistent with previous studies that used FRAP histochemistry in combination with sensory neuron markers (Hunt and Mantyh, 2001; Carr et al., 1990).

Predominant expression of the transmembrane isoform of PAP in DRG is consistent with ultrastructural studies (Csillik and Knyihar-Csillik, 1986) showing that FRAP is localized to the membrane of small-diameter DRG neurons. Thus, TM-PAP and FRAP share the same cellular and subcellular localization in DRG neurons (membrane associated) further suggesting PAP encodes FRAP. When taken together, these findings solve a fifty-year-old mystery, and demonstrate that FRAP in nociceptive neurons is equivalent to PAP.

TABLE 2
Quantitative analysis of PAP and sensory neuron marker
colocalization studies within mouse L4-L6 DRG neurons. The
percentage of cells that co-express the indicated
markers ± SEM is shown.

| | Percentage of PAP⁺ | Percentage of |
|---|---|---|
| | neurons expressing | marker⁺ neurons |
| Marker | indicated marker | expressing PAP |
| IB4 | 70.6 ± 3.8 | 91.6 ± 2.8 |
| Mrgprd-EGFPf | 66.2 ± 3.2 | 99.2 ± 0.8 |
| P2X3 | 84.5 ± 6.1 | 92.6 ± 3.1 |
| TRPV1 | 19.1 ± 1.3 | 14.4 ± 1.3 |
| CGRP | 16.9 ± 3.9 | 17.1 ± 3.2 |

### Example 4

### Role of PAP in Pain Sensory Mechanisms

Microarray analysis has demonstrated that numerous genes are up- or down-regulated in rat DRG three days after sciatic nerve transection (Costigan et al., 2002) and following nerve injury in a neuropathic pain model (Davis-Taber, 2006). The microarray dataset presented in Costigan et al. (presented in Costigan et al. as Supplemental FIG. 2) was reanalyzed and all 241 genes ranked by expression fold change (because the genes were listed in alphabetical order, which is biologically meaningless). The re-analysis revealed that PAP mRNA is down-regulated 3.5-fold after sciatic nerve transection and is the second most down-regulated gene overall. See Table 3. Similarly, PAP mRNA is one of the most heavily down-regulated genes in a neuropathic pain model (Davis-Taber, 2006). Since PAP expression is down-regulated in these animal models of neuropathic pain, neuropathic pain could be treated by restoring PAP activity.

TABLE 3
Top five genes down-regulated in rat DRG three
days post sciatic nerve transaction.

| | Gene | | |
|---|---|---|---|
| Rank | Symbol | Name | Fold Change |
| 1 | IAPP | Islet amyloid polypeptide (related to | −4.72* |
| | | CGRP) | |
| 2 | PAP(Acpp) | Acid phosphatase, prostate | −3.56 |
| 3 | Ass1 | Argininosuccinate synthetase | −3.31 |
| 4 | Mrpl13 | Mitochrondrial ribosomal protein L13 | −2.95 |
| 5 | Doc2a | Double C2, alpha | −2.71 |

*Independently validated by Mulder et al.; who showed that IAPP was down-regulated in DRG upon sciatic nerve transection (Mulder et al., 1997).

### Example 5

### DRG Neurons Express LPA Receptors

Expression of LPA receptors was analyzed in DRG neurons to confirm a role for PAP in regulation of LPA receptor signaling. At the time these studies were begun, RT-PCR experiments indicated that LPA1 was the only LPA receptor in DRG (Inoue et al., 2004; Renback et al., 2000). To examine expression of these receptors in more detail, in situ hybridization was performed with antisense LPA1 and LPA3 riboprobes. These experiments revealed that LPA1 was expressed in all mouse DRG neurons while LPA3 was expressed in a subset of small diameter DRG neurons. To determine if LPA3 was co-expressed with Mrgprd, fluorescent double in situ hybridization was performed with antisense Mrgprd and LPA3 riboprobes using previously published methods (Zylka et al., 2003). The experiment revealed that all Mrgprd+ neurons expressed LPA3. Conversely, almost all LPA3+ cells expressed Mrgprd (although there were a few LPA3+ only cells). In summary, all DRG neurons express LPA1 while Mrgprd+ neurons co-express LPA1 and LPA3. These data suggest that all DRG neurons have the potential to signal via LPA receptors. Since Mrgprd+ neurons also express PAP (see Table 2), LPA receptor signaling can be modulated by increasing and decreasing PAP protein levels.

### Example 6

### Quantitative Fluorometric Assay for Measuring PAP Activity in Solution

A way to quantify PAP activity was needed so that reproducible amounts of active PAP protein could be added to cultured cells or injected into live mice for the experiments described below. To accomplish this, two well-established methods were tested for measuring PAP activity: 1) a colorimetric assay using para-nitrophenyl phosphate (p-NPP) hydrolysis; and 2) a fluorometric assay using difluoro-4-methylumbelliferyl phosphate (DiFMUP) hydrolysis (commercially available as the EnzChek Acid Phosphatase kit from Invitrogen, Carlsbad, Calif., United States of America). Based on direct comparisons, it was determined that the fluorometric assay was much more sensitive than p-NPP for quantification of PAP activity. Exemplary data with purified bovine PAP (bPAP, secreted isoform) and mouse PAP (mPAP) are presented in FIG. 3. PAP phosphatase activity is inhibited by L-tartrate, a well-characterized PAP inhibitor (Ostrowski and Kuciel, 1994). Importantly, this assay can be used to determine enzyme activity (units/mg protein) by generating standard curves. This fluorometric assay can thus be used to quantify phosphatase activity of pure PAP protein and PAP from cell lysates.

### Example 7

### Bovine PAP Dephosphorylates LPA and Inhibits LPA-Evoked Signaling

Previous studies found that human PAP dephosphorylates LPA in test tubes (Hiroyama and Takenawa, 1999; Tanaka et al., 2004). Although it is assumed that dephosphorylated LPA can no longer activate LPA receptors, this was never formally demonstrated using more biologically-meaningful, cell-based assays. To prove that PAP inactivates LPA, 1 μM LPA was incubated with an excess (0.2 mU) of bovine PAP in a test tube for 1.5 hr at 37° C. (“a” in FIG. 4). In parallel, a second tube was incubated containing 1 μM LPA (without bPAP) for 1.5 hr at 37° C. (“b” in FIG. 4). Rat1 cells were loaded with the calcium-sensitive dye Fura2-acetoxymethyl (AM) ester (Dong et al., 2001), and (LPA+bPAP) applied to these cells for 1 minute (see “a” in FIG. 4). Following a brief washout period, (LPA) was applied to the cells for 1 minute (see “b” in FIG. 4). As can be seen in FIG. 4, intracellular calcium levels did not appreciably change when Rat1 cells were stimulated with LPA+bPAP; however, intracellular calcium levels dramatically changed when these same cells were stimulated with LPA. These data clearly indicate that bPAP dephosphorylates and inactivates LPA. Such inactivation effectively inhibits LPA-evoked signaling. Since bPAP and hPAP are commercially available (Sigma, St. Louis, Mo., United States of America), these pure proteins were use ed in a non-genetic approach to increase PAP activity in the experiments described below.

### Example 8

### mPAP Acutely Reduces LPA-Evoked Calcium Responses in Rat1 Fibroblasts

Since exogenous bPAP could block LPA-evoked signaling, it was hypothesized that LPA-evoked signaling could be acutely reduced in Rat1 cells that over-expressed PAP. To test this hypothesis, a fluorescently tagged mPAP construct was generated by fusing the yellow fluorescent protein Venus to the C-terminus of TM-PAP (Nagai et al., 2002). This allowed direct visualization of live cells that were transfected with PAP-Venus. It was demonstrated that PAP-Venus had phosphatase activity by staining transfected cells using FRAP histochemistry. The catalytically active fusion construct was then transfected into Rat1 cells and LPA-evoked changes in intracellular calcium were measured with the calcium-sensitive dye Fura2-AM. As can be seen in FIG. 5, the LPA-evoked calcium response amplitude and duration are acutely reduced in cells transfected with PAP-Venus relative to untransfected cells. This indicates that mouse PAP acutely reduces LPA-evoked signaling in a cell-based context. These findings, combined with published results, indicate that mouse, cow and human PAP dephosphorylate LPA. This suggests a highly conserved function for PAP.

### Example 9

### LPA Response is Dependent on PAP Phosphatase Activity

To support the hypothesis that PAP modulates LPA signaling by dephosphorylating LPA, a phosphatase-dead mouse PAP expression construct (PAP-mutant) was engineered by mutating the active site residue Histidine 12 to Alanine, and then fusing the fluorescent protein Venus to the C-terminus (to permit visualization of cells transfected with this PAP-mutant). First, it was confirmed that the PAP mutant construct was expressed and membrane localized as effectively as wild-type PAP-Venus. Second, it was confirmed that the PAP-mutant construct lacked phosphatase activity using Fluoride-Resistant Acid Phosphatase (FRAP) histochemistry. Then, Rat1 fibroblasts were transfected with PAP or PAP-mutant, and the cells loaded with the calcium-sensitive dye Fura2-AM. The cells were then stimulated with 100 nM LPA. Calcium responses were compared in PAP transfected cells to untransfected cells in the same field of view. As can be seen in FIGS. 6A and 6C, the LPA-evoked calcium response was significantly reduced in PAP transfected cells, reproducing results presented in FIG. 5. In contrast, LPA-evoked calcium responses were not altered in cells transfected with the phosphatase-dead PAP-mutant. See FIGS. 6B and 6D. These results indicate that the reduced LPA response in PAP transfected cells shown in FIGS. 6A and 6C is dependent on PAP phosphatase activity.

### Example 10

### Use of PAP for Pain Treatment

An abnormal amount of LPA stimulates the nociceptive system and initiates neuropathic pain including allodynia and hyperalgesia. See FIG. 7. Neuropathic pain could be treated by increasing LPA phosphatase activity (FIG. 7). The data described herein above indicate that PAP is capable of degrading LPA and reducing LPA-evoked signaling. Thus, PAP injections can regulate LPA-evoked signaling in several cell types (neurons, microglial cells, Schwann cells) that are implicated in neuropathic pain and have additional effects, such as blocking LPA-evoked signaling in Schwann cells and blocking demyelination. These possibilities can be tested by imaging sciatic nerve using electron microscopy (as performed in (Zylka et al., 2005)), then measuring myelin thickness in control and treated animals.

In addition, PAP expression and FRAP activity are down-regulated after nerve injury. Accordingly, injection of PAP after nerve injury can restore PAP activity and reduce allodynia during the maintenance phase of neuropathic pain. See FIG. 8. Neuropathic pain can be treated by reducing LPA concentrations in spinal cord and blocking initiation or maintenance of a chronic pain condition. One method of degrading high concentrations of LPA is through injection of pure PAP protein directly into the spinal cord (intrathecal injection) before or following nerve injury. See FIG. 8. By injecting a bolus of PAP protein into the spinal cord, PAP can degrade LPA that is released post-injury. This effectively inhibits LPA receptor signaling and blocks thermal and mechanical sensitization in mice after nerve injury. Alternatively, PAP can be injected intravenously or delivered directly to the site of nerve injury (via intramuscular injection or mini-pump). Additional methods for increasing PAP in the nociceptive system include administration of a PAP agonist and administration of PAP using gene therapy or stem cell approaches. See FIG. 9.

### Example 11

### PAP Inhibition of Allodynia and Hyperalgesia in Vivo

Dose selection. An initial dose of 100 mU PAP intrathecally (i.t.) was chosen based on the finding that 1 μmol of fluorometric substrate is degraded by 1 U of bovine PAP per minute. If it is assumed that bPAP hydrolyzes the fluorometric substrate as efficiently as LPA, then this equals a rate of 1 μmol of LPA hydrolyzed/U bPAP/minute. LPA (1 nmol, i.t.) caused behavioral allodynia and hyperalgesia that was equal in magnitude to that seen after nerve injury (Inoue et al., 2004). If it is assumed that a similar amount of LPA is released by platelets after nerve injury, then to degrade 1 nmol LPA in 1 minute, 1 mU of bPAP would be required. Thus, a 100 mU dose of PAP represents 100-fold excess, and accounts for diffusion and dilution in CSF and spinal cord parenchyma.

The direct lumbar puncture method was used to intrathecally (i.t.) inject 5 μL of approximately 100 mU PAP (Sigma, St. Louis, Mo., United States of America) dissolved in 0.9% saline between the lumbar 5 and 6 regions of mouse spinal cord (Fairbanks, 2003). Intrathecal injection was chosen because PAP protein is unlikely to reach spinal cord tissue if injected intraperitoneally. Bovine serum albumin was purchased from Sigma (St. Louis, Mo., United States of America, Catalog Number P8361, expressed in Pichia pastoris, >4000 U/mg protein). Morphine sulfate (Sigma, St. Louis, Mo., United States of America, Catalog Number M8777) was diluted into 0.9% saline.

Intrathecal injection of bPAP or hPAP had no obvious side effects. For example, no paralysis, muscle weakness, lethargy, excitability, infection or death was observed for the duration of the behavioral testing period (up to 14 days in some cases). It was expected that bPAP and hPAP protein would be well tolerated in vivo, because PAP protein is located extracellularly in the spinal cord (on the axons of PAP+ neurons). In addition, because PAP was being injected into the CNS (i.e. behind the blood-brain-barrier), and the CNS is immune privileged, an immune response seemed unlikely. Signs of immune and microglial activation can be monitored using molecular markers.

PAP activity can also be increased using additional methods such as by plasmid or viral transduction, or by injecting cell lines that over-express the secreted isoform of PAP.

PAP can be inactivated by heat-denaturation, DEPC-treatment or by introducing a catalytically inactive point mutation (His12→Ala) into recombinant protein.

bPAP inhibits LPA-evoked sensitization in vivo. To prove that bovine PAP protein (bPAP) (purchased from Sigma, St. Louis, Mo., United States of America) is non-toxic when injected i.t., and to prove that bPAP can modulate LPA-evoked signaling in vivo, four groups of wild-type C57BL/6 male mice were injected (i.t.) with: 1) vehicle, 2) 20 μU bPAP, 3) 1 nmol LPA, or 4) 1 nmol LPA+20 μU bPAP. It was found that 20 μU bPAP could dephosphorylate 1 nmol LPA when incubated at 37° C. for 10 min.; therefore, all samples were incubated at 37° C. for 10 min. prior to injection.

First, mechanical sensitivity was measured with an electronic von Frey apparatus (IITC). Then, thermal sensitivity was measured using the Hargreaves method (radiant heating of hindpaw; IITC Plantar Test Apparatus). As can be seen in FIG. 10, 1 nmol LPA caused long-lasting mechanical allodynia and thermal hyperalgesia, as was previously reported (Inoue et al., 2004). When 1 nmol LPA was incubated with bPAP for 10 min. at 37° and then injected, no behavioral sensitization to LPA was observed. In principle, the data in FIG. 10 demonstrate that bPAP is competent to degrade LPA and inhibit LPA-evoked signaling in vivo. Surprisingly, it was found that thermal sensitivity was significantly increased for three days in bPAP-injected mice compared to vehicle-injected mice. See FIG. 10B.

This significant increase in thermal sensitivity was reproduced with additional vehicle- and bPAP-injected mice. Mechanical sensitivity in these same animals was not significantly different when compared to vehicle controls (with the exception of the 6 hour time point). These findings show that bPAP has analgesic properties in vivo.

No significant thermal analgesia was observed in LPA+bPAP-injected mice (except at the 1 day time point). This difference between LPA+bPAP-injected mice and bPAP-injected mice could be due to incomplete dephosphorylation of LPA prior to injection or could be due to the presence of monoglyceride and inorganic phosphate in the LPA+bPAP sample (dephosphorylation of LPA produces monoglyceride and inorganic phosphate). Body weight was stable for the entire experimental period indicating no loss of appetite or infection. Overall, these experiments indicate that i.t. injection of bPAP is non-toxic and well-tolerated in mice.

bPAP and hPAP are analgesic in vivo. To determine pain-related functions for PAP, bovine bPAP was injected into spinal cord of wild-type mice. These mice were then tested before and up to 5 days post injection for thermal sensitivity using the Hargreave's method (radiant heating of hindpaw) and mechanical sensitivity using an electronic Von Frey apparatus. Mice injected with bPAP showed significantly increased latency to withdraw their hindpaws from the thermal stimulus for up to 3 days compared to vehicle-injected controls. See FIG. 11A, compare dashed line to solid line. In contrast, there were no significant differences (except at the 6 hr time point) in mechanical sensitivity. See FIG. 11B. Note that data in FIGS. 11A and 11B are taken from FIGS. 10A and 10B and re-plotted to facilitate comparison with hPAP behavioral results. The data, combined with the fact that bPAP injections did not cause paralysis or lethargy, strongly suggests that PAP is analgesic, not paralytic or hypnotic. Moreover, intrathecal injection of human hPAP also caused significant thermal analgesia, but not mechanical analgesia, for 3 days following injection. See FIGS. 11C and 11D. The hPAP preparation was dialyzed against 0.9% saline before injection, so this analgesic effect was unlikely to be due to a small-molecule contaminant in the protein preparation. Moreover, the fact that bovine and human PAP produced similar analgesic effects with similar duration, further suggests this effect is specific to PAP. Analgesia was not observed when Bovine Serum Albumin (BSA) was injected. See FIGS. 11C and 11D. BSA is a protein that is similar in molecular weight to PAP but lacks phosphatase activity. Further, no thermal or mechanical sensitivity alteration was observed following i.t. injection of a different secreted phosphatase, i.e., bovine alkaline phosphatase. See FIGS. 12A and 12D.

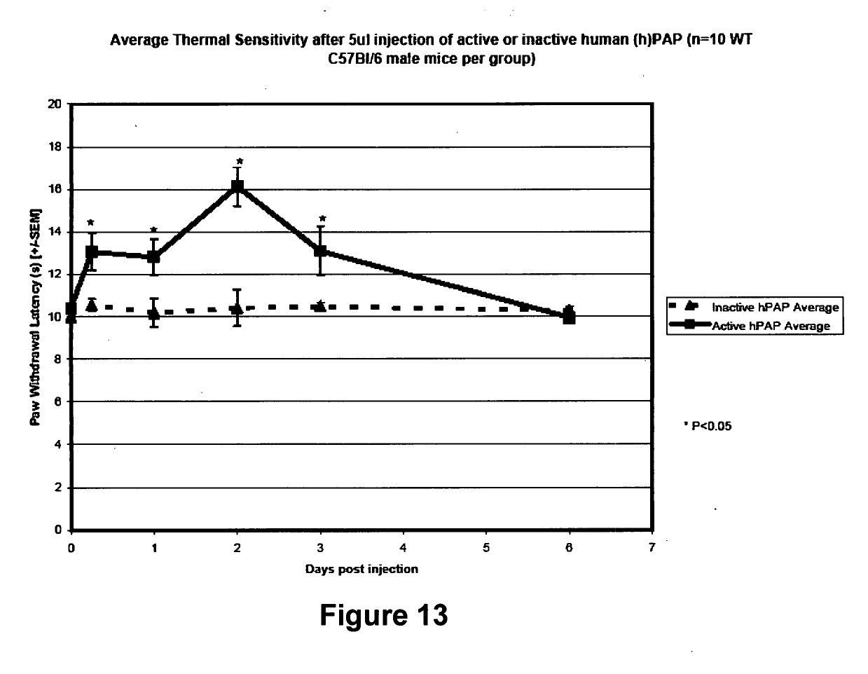

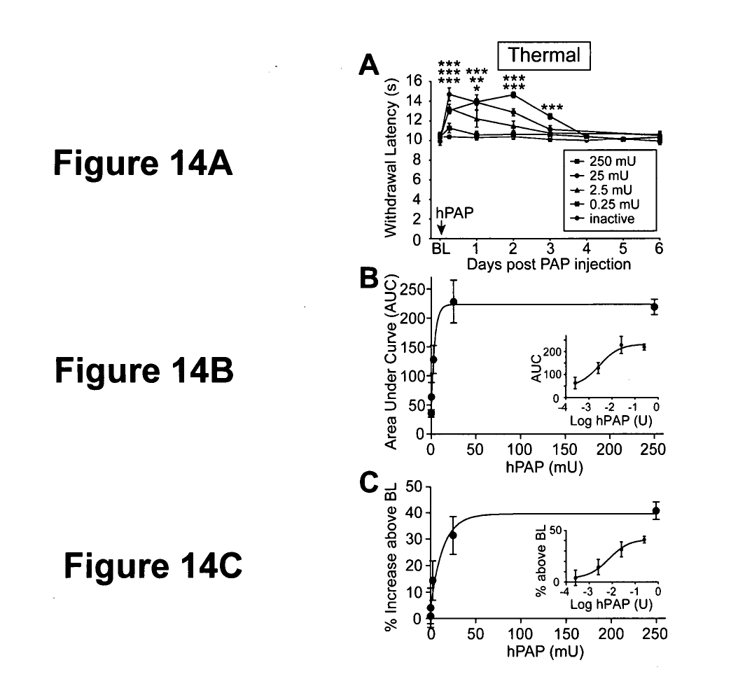

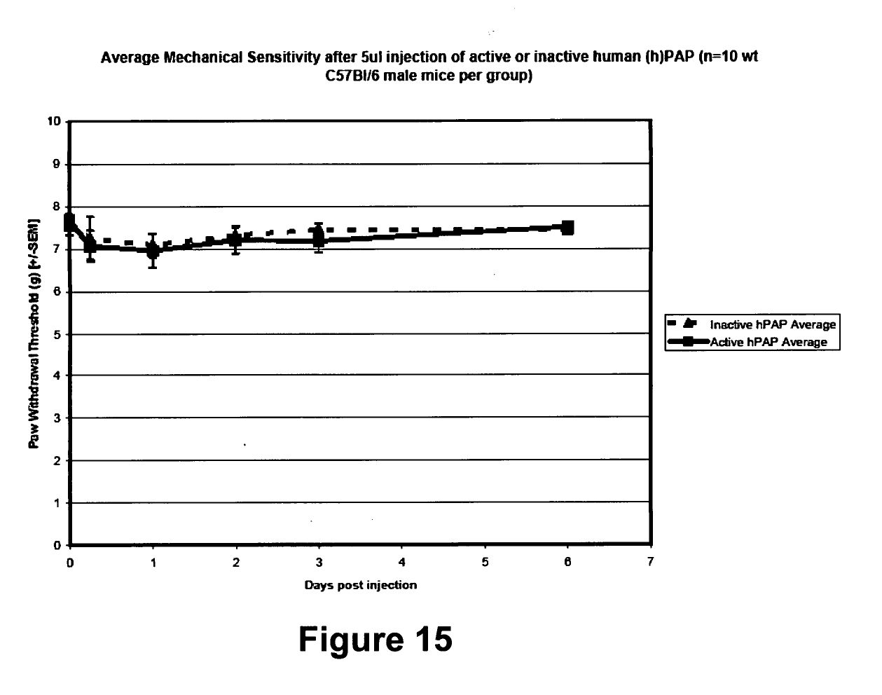

Active and heat-inactivated hPAP were used to directly test if PAP catalytic activity is required for the analgesic effect. FIG. 13 shows the average thermal sensitivity of 10 wild-type C57BL/6 male mice for 6 days after i.t. injection of 5 μl of active (solid line) or inactive (dashed line) hPAP. The antinociceptive effect of active hPAP was dose dependent. See FIGS. 14A-14C. FIG. 15 shows the average mechanical sensitivity of 10 wild-type C57BL/6 male mice for 6 days after i.t. injection of 5 μl of active (solid line) or inactive (dashed line) hPAP. Again, intrathecal injection of human hPAP caused significant thermal analgesia, but not mechanical analgesia, for 3 days following injection.

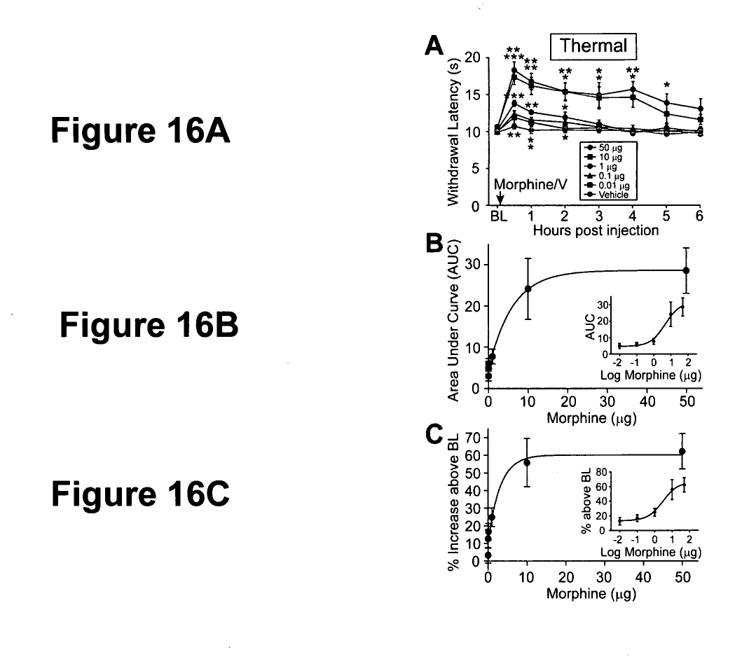

Next, PAP antinociception was compared to the commonly used opioid analgesic morphine using the same behavioral assay for sensitivity to a noxious thermal stimulus. The dose dependency of morphine antinociception is shown in FIGS. 16A-16C. Comparing the data in FIGS. 14A-14C to the data in FIGS. 16A-16C, PAP and morphine antinociception appear to be similar in magnitude following a single i.t. injections (40.8%±3.3% versus 62.2%±9.9% increase above baseline at the highest doses, respectively) but the PAP antinociception lasted much longer than morphine (3 days verses 5 hr at the highest doses, respectively. Previous reports found that the same high dose of morphine (50 μg, i.t., single injection) lasted 4.6±1.0 hr in mice (Grant et al., 1995).

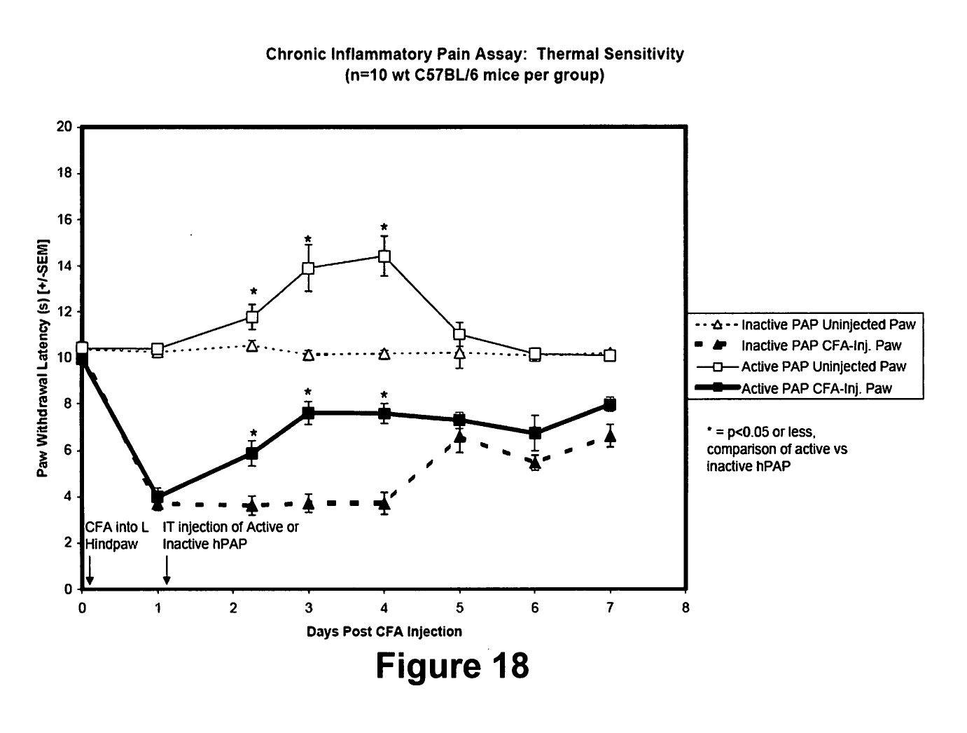

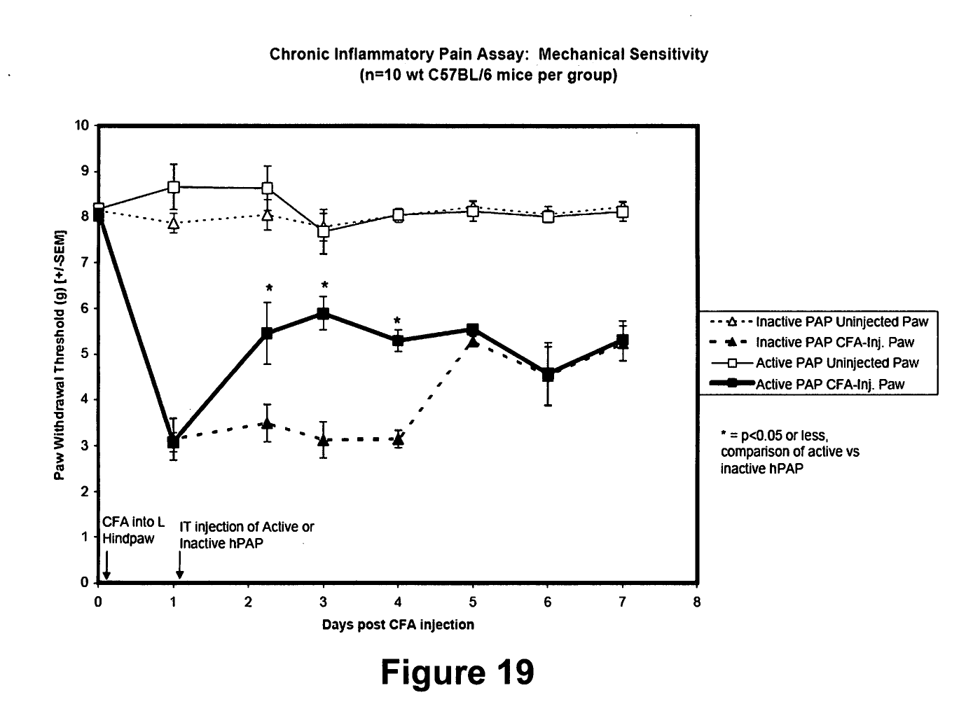

Complete Freund's Adjuvant (CFA) inflammatory pain model. The Complete Freund's Adjuvant (CFA) inflammatory pain model was used to determine if PAP could reverse chronic mechanical and thermal inflammatory pain. The baseline mechanical sensitivity of adult (2-3 months old), age-matched, weight-matched male C57BL/6 mice was quantified by probing glabrous skin (right hindpaw) with an electronic von Frey apparatus (IITC). The Hargreave's method, which entails radiant heating of the hindpaw (IITC Plantar Test Apparatus), was used to test thermal sensitivity in the same group of mice (Hargreaves et al., 1988). Baseline thermal and mechanical sensitivity was determined prior to injection of test compounds. The mice were then injected with 20 μL CFA. One day later, all mice showed profound thermal and mechanical hypersensitivity in the CFA-injected hindpaw. Half of the mice were then intrathecally injected with 1.3 mg/mL BSA (control) and the other half with bPAP (see FIGS. 17A and 17B) or half with active hPAP and half with inactive hPAP. See FIGS. 18 and 19. Mice were then tested for mechanical and thermal sensitivity up to 7 days post injection, using von Frey and Hargreaves tests. Average sensitivity was plotted and statistical tests (paired t-test) were used to determine if PAP causes hypersensitivity (allodynia; hyperalgesia), hyposensitivity (analgesia), or has no effect.

Strikingly, bPAP significantly reversed inflammatory pain caused by thermal and mechanical stimuli. See FIGS. 17A and 17B. The same effect was observed for injection of active hPAP. See FIGS. 18 and 19. This analgesic effect lasted for at least three days. This indicates that a single dose of PAP is able to treat chronic pain to the point that mice almost fully recover.

PAP treatment of neuropathic pain. The extent to which intrathecal injection of PAP protein can block maintenance of neuropathic pain was determined. The main difference between blocking initiation and maintenance of neuropathic pain has to do with when PAP is injected relative to the spared nerve injury (SNI) surgery. See FIG. 8. Injection of PAP before nerve injury measures effectiveness at blocking initiation of neuropathic pain while injecting PAP 4-5 days after injury tests effectiveness at blocking maintained pain. The SNI model was used because peripheral nerve injury most closely models human neuropathic pain in terms of symptoms and responsiveness to drugs (Abdi et al., 1998; LaBuda and Little, 2005).

The spared nerve injury (SNI) model was used to produce a neuropathic-like pain state in mice. Surgeries were performed in the animal facility following published procedures (Shields et al., 2003). In brief, mice were anesthetized with halothane, the sural and peroneal branches of the right sciatic nerve were ligated, then ˜1 mm from each nerve cut. The tibial nerve was spared. This causes profound mechanical allodynia in the right hindpaw but little thermal hyperalgesia (Shields et al., 2003).

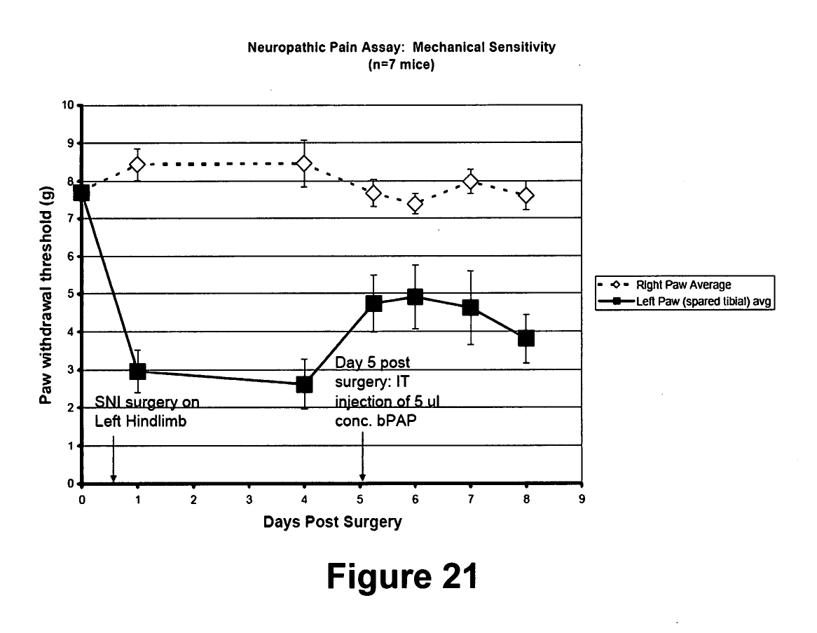

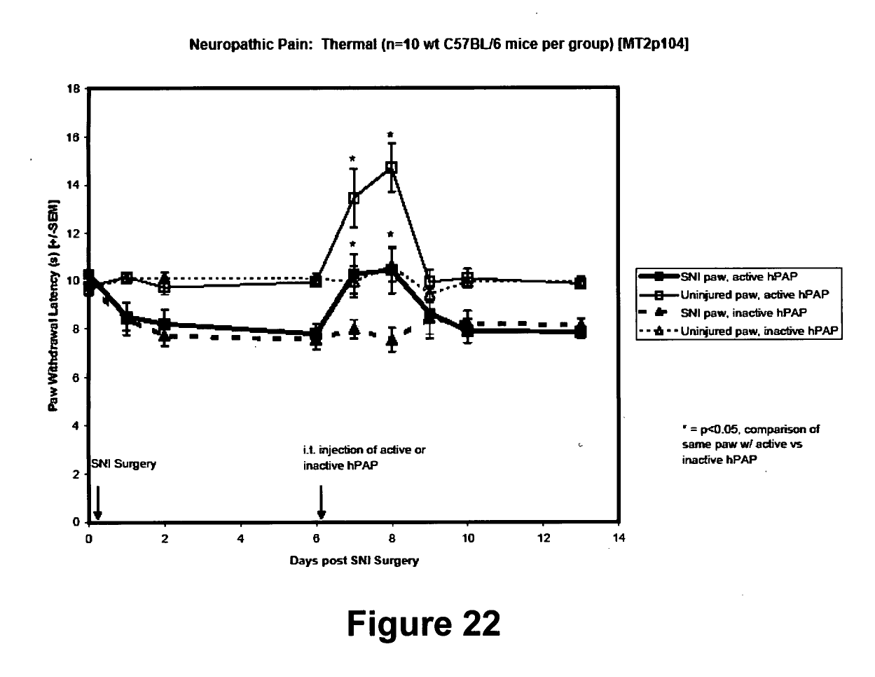

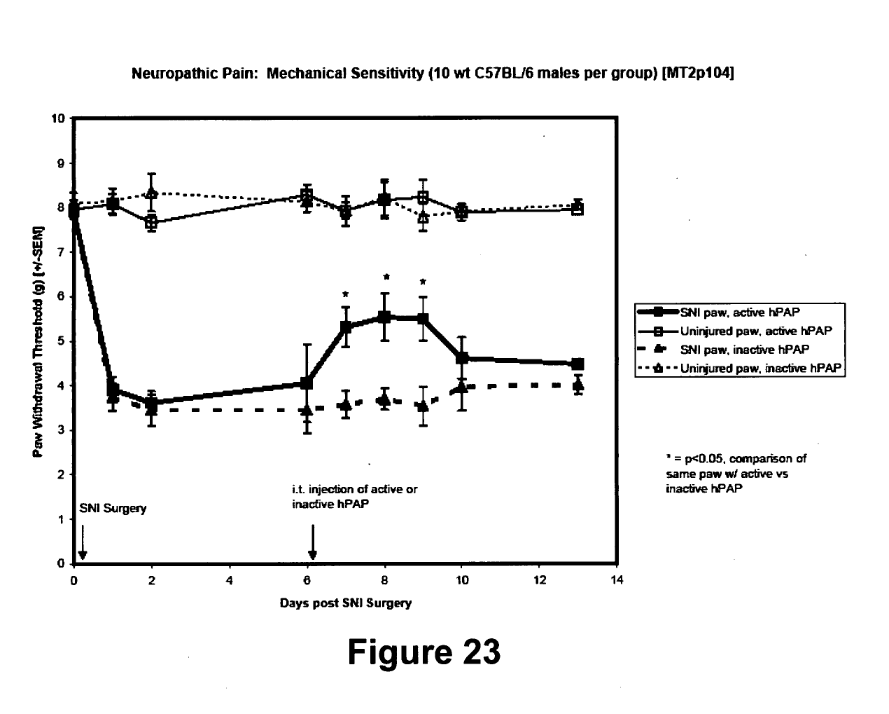

The right (control-untreated) and left (injured) hindpaws were tested for mechanical sensitivity (using the von Frey method; described above) and thermal sensitivity (Hargreave's method; described above) before surgery (baseline) and post SNI-surgery. Active bPAP or hPAP was injected i.t. using a dose that was empirically found to have maximal phosphatase activity but minimal side effects. An equivalent amount of inactive hPAP protein was injected to prove that the observed analgesic effects were due to PAP phosphatase activity. Injections (i.t.) were performed as described above 5-6 days after surgery (maintenance experiments). Statistical tests (t-tests) were used to determine the significance of differences in thermal and/or mechanical sensitivity between control and experimental animals. For the injured paw, i.t. injection of bPAP caused a decrease in thermal (see FIG. 20) and mechanical (see FIG. 21) sensitivity lasting for about 3 days. For the uninjured paw, decreased sensitivity was only observed in thermal and not mechanical sensitivity. Very similar results on thermal sensitivity (see FIG. 22) and mechanical sensitivity (see FIG. 23) were observed for intrathecal injection of active hPAP.

These data suggest chronic pain can be treated in humans and other animal subjects by intrathecally injecting purified PAP protein or by administering small-molecule allosteric modulators to activate PAP normally present on pain-sensing neurons. These drug treatments can be used pre- or post-operatively to treat surgical pain; to treat chronic inflammatory pain (e.g., osteoarthritis, burns, joint pain, lower back pain); and to treat chronic neuropathic pain.

### Example 12

### PAP Inhibition of Alloydynia and Hyperalqesia in PAP Knockout Mice

PAP was generally thought to function only in the prostate (Ostrowski and Kuciel, 1994). However, the presently disclosed data suggests that PAP can also function in nociceptive neurons. To further evaluate pain-related functions for PAP, age-matched wild-type C57BL/6 and PAP^{-/-} male mice (backcrossed to C57BL/6 for 10 generations) were evaluated using acute and chronic pain behavioral assays. No significant differences between genotypes were found using a measure of mechanical sensitivity (electronic von Frey) or several different measures of acute noxious thermal sensitivity. See Table 4.

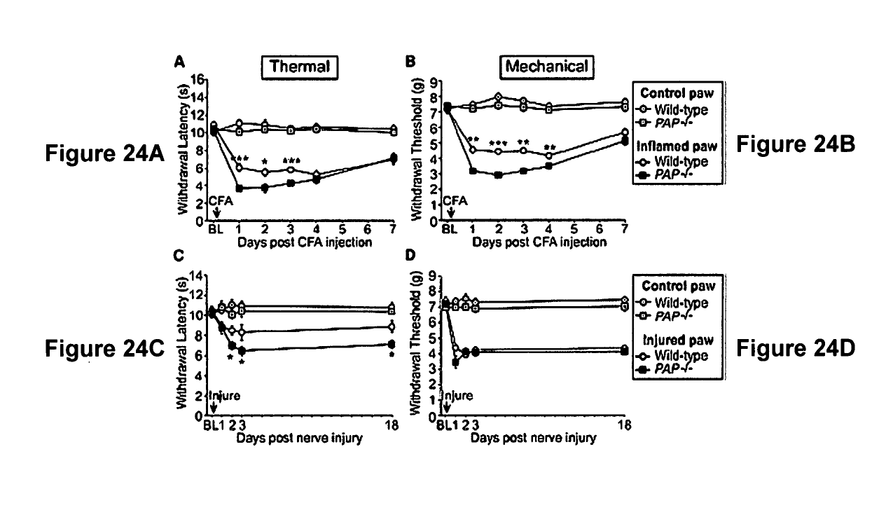

In contrast, PAP^{-/-} mice showed significantly greater thermal hyperalgesia and mechanical allodynia relative to wild-type mice in the Complete Freund's Adjuvant (CFA) model of chronic inflammatory pain. See FIGS. 24A and 24B. In addition, PAP^{-/-} mice showed significantly greater thermal hyperalgesia in the spared nerve injury (SNI) model of neuropathic pain (Shields et al., 2003). See FIG. 24C.

TABLE 4
Acute mechanical and thermal sensitivity are normal in PAP^{-/-}
mice.

| | | |
|---|---|---|
| Behavioral Assay | Wild-type | PAP^{-/-} |

Withdrawal threshold:

| | | |
|---|---|---|
| Electronic von Frey | 7.2 ± 0.4 g | 7.8 ± 0.5 g |

Withdrawal latency:

| | | |
|---|---|---|
| Radiant heating of hindpaw | 9.1 ± 0.7 s | 9.9 ± 0.9 s |
| (Hargreaves Method) | | |
| Tail immersion at 46.5° C. | 18.4 ± 2.8 s | 16.4 ± 1.6 s |
| Tail immersion at 49.0° C. | 9.9 ± 0.7 s | 9.8 ± 0.9 s |
| Hot plate at 52° C. | 20.0 ± 1.1 s | 19.3 ± 1.3 s |

Data are expressed as means ± s.e.m.
There were no significant differences between genotypes in any of the listed behavioral assays, paired t-test, P > 0.05. n = 10 male mice tested per genotype for all assays except hotplate and tail immersion at 49° C. For these latter two assays, n = 14 mice (8 females, 6 males) were tested per genotype.

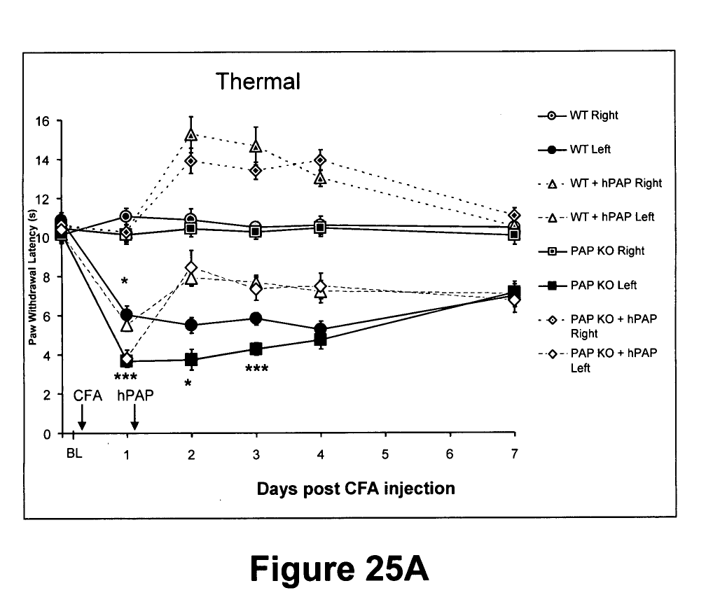

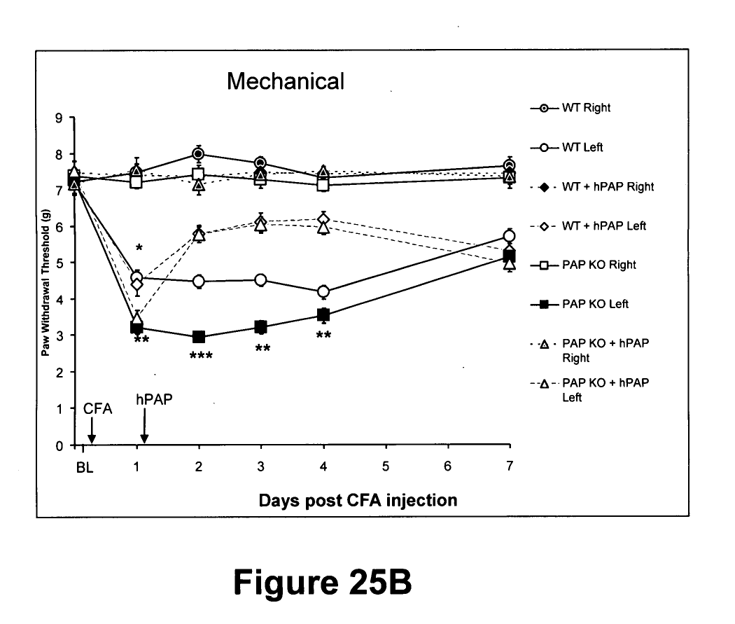

Since PAP^{-/-} mice showed enhanced hyperalgesia and allodynia in the CFA inflammatory pain model, the ability of hPAP treatment to rescue these enhanced thermal and mechanical phenotypes in PAP^{-/-} mice was examined. Intrathaceal injection of hPAP increases thermal withdrawal latency in the control (right) paw of PAP^{-/-} (PAP KO) mice to the same extent as wild-type mice. See FIG. 25A. Thus it appears that PAP^{-/-} mice are competent to respond to acute increases in PAP activity. Strikingly, injection of hPAP rescues the thermal and mechanical inflammatory pain phenotype in the inflamed (left) paw of PAP^{-/-} mice. See FIGS. 25A and 25B, compare data for active PAP versus inactive PAP. Localized, spinal injection of hPAP can rescue the behavioral deficit caused by deletion of PAP throughout the animal.

### Example 13

### PAP Generation of Adenosine

The anti-nociceptive effects of PAP require catalytic activity. Without being bound to any one theory, this suggests that PAP generates, via dephosphorylation, a molecule that regulates nociceptive neurotransmission in the spinal cord. PAP and TMPase can dephosphorylate many different substrates (Dziembor-Gryszkiewicz et al., 1978; Sanyal and Rustioni, 1974; Silverman and Kruger, 1988b; Vihko, 1978b). One possible substrate is AMP. Dephosphorylation of AMP produces adenosine, a molecule that inhibits nociceptive neurotransmission in spinal cord slices and has well-studied analgesic properties in mammals (Li and Perl, 1994; Liu and Salter, 2005; Post, 1984; Sawynok, 2006).

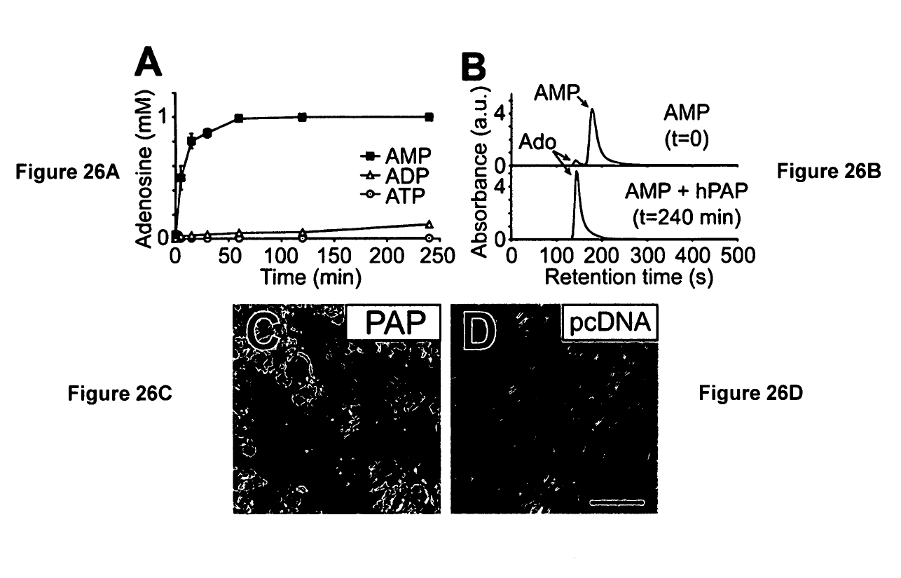

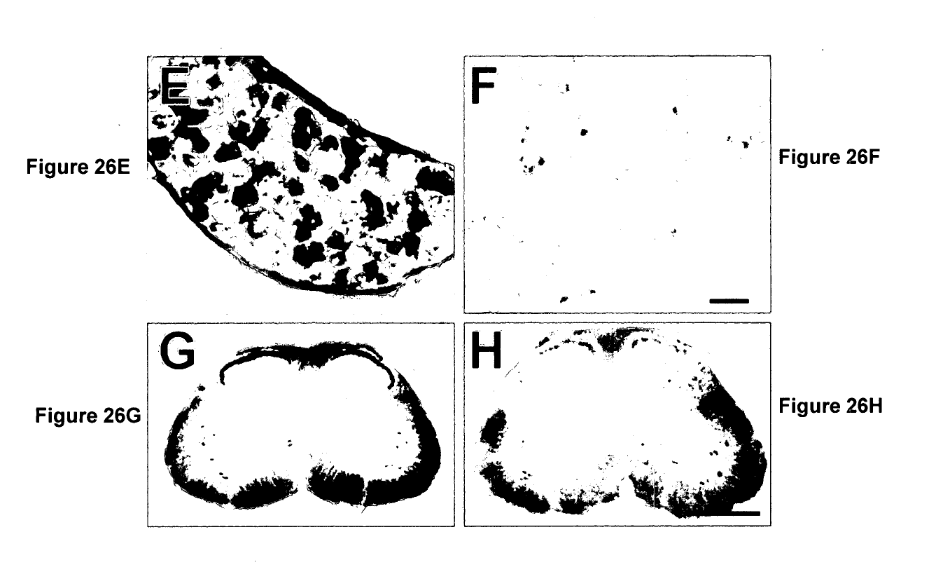

Prior to the presently disclosed subject matter, there was no direct proof that PAP or TMPase could generate adenosine from AMP. Instead, production of adenosine was inferred by measuring production of inorganic phosphate (Vihko, 1978b). To directly test whether PAP could generate adenosine from AMP and other adenine nucleotides, PAP was incubated with 1 mM AMP, ADP or ATP at pH 7.0 for 4 h. Adenine nucleotides and adenosine were detected using high performance liquid chromatography (HPLC) and UV absorbance (Lazarowski et al., 2004). These studies revealed that PAP can rapidly dephosphorylate AMP and, to a much lesser extent ADP, to adenosine. See FIGS. 26A and 26B. Importantly, no unexpected peaks were seen in the chromatograms, ruling out the possibility that PAP had additional hydrolytic activities towards nucleotides.

Next, the extent to which PAP could dephosphorylate extracellular AMP in HEK 293 cells, DRG neurons and spinal cord was studied using AMP enzyme histochemistry. HEK 293 cells transfected with TM-PAP were heavily stained whereas control cells were not (see FIGS. 26C and 26D), highlighting that TM-PAP dephosphorylates extracellular AMP and hence has ecto-5′-nucleotidase activity. In addition, small-diameter DRG neurons from wild-type mice were intensely stained while large-diameter neurons had weak granular cytoplasmic staining. In contrast, only weak granular staining was present in DRG neurons from PAP^{-/-} mice. See FIGS. 26E and 26F. These data indicate that PAP is the predominant ecto-5′-nucleotidase on the soma of small-diameter neurons. Lastly, AMP histochemical staining of axon terminals in lamina II was reduced in PAP^{-/-} relative to wild-type mice, but was not eliminated. See FIGS. 26G and 26H. This indicates that PAP is one of perhaps many enzymes in spinal cord with the ability to dephosphorylate AMP to adenosine.

Adenosine mediates anti-nociception through Gᵢ-coupled A₁-adenosine receptors (A₁Rs) (Lee and Yaksh, 1996; Sawynok, 2006). To directly test whether A₁Rs were required for PAP anti-nociception, wild-type C57BL/6 and A₁-adenosine receptor knockout mice (A₁R^{-/-}, Adora1^{-/-};

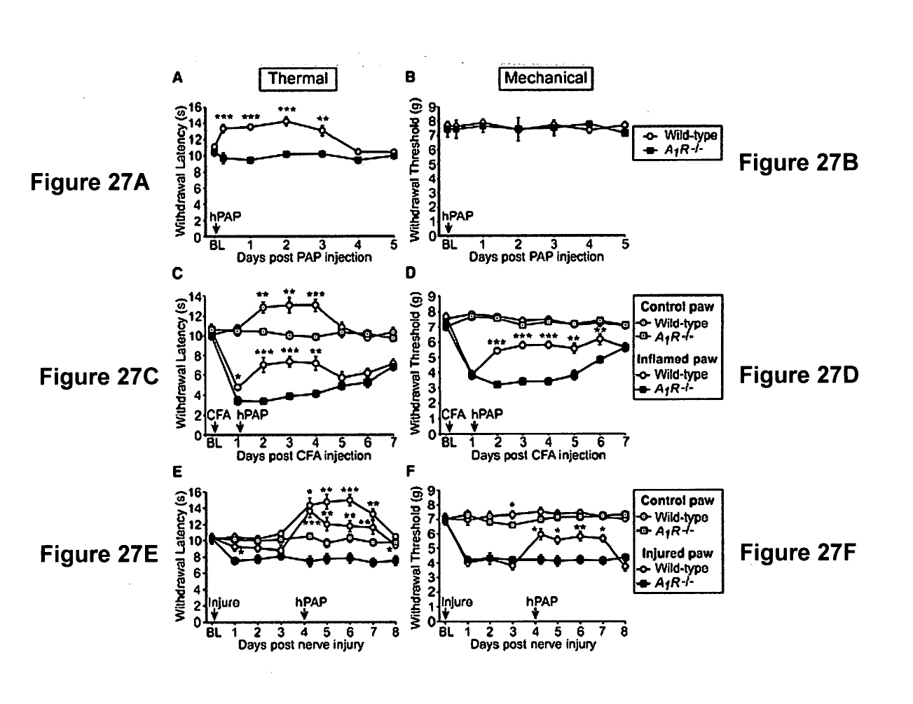

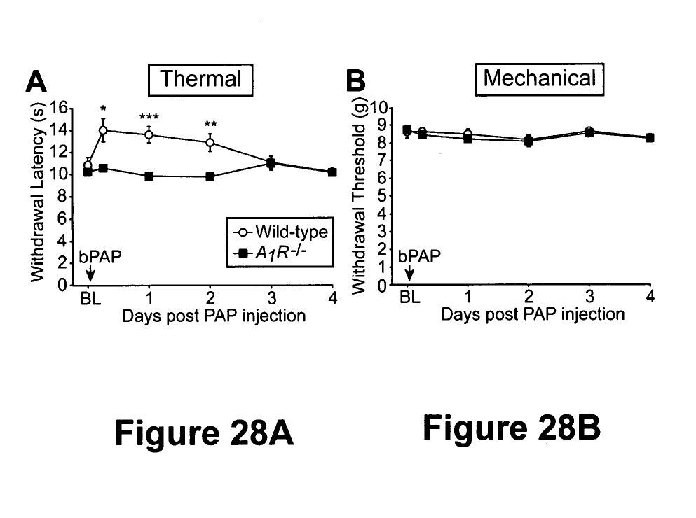

backcrossed to C57BL/6 mice for 12 generations), were i.t. injected with hPAP. Then noxious thermal and mechanical sensitivity was measured (Hua et al., 2007; Johansson et al., 2001). Strikingly, hPAP increased thermal paw withdrawal latency for three days in wild-type mice but was without effect in A₁R^{-/-} mice. See FIG. 27A. Similarly, bPAP increased paw withdrawal latency to the noxious thermal stimulus in wild-type mice but had no effect in A₁R^{-/-} mice. See FIG. 28. As expected, hPAP did not affect mechanical sensitivity in uninjured animals. See FIG. 27B.

The responses of wild-type and A₁R^{-/-} mice were also tested using the CFA chronic inflammatory pain model and the SNI neuropathic pain model. Reproducing previous findings (Wu et al., 2005), A₁R^{-/-} mice showed greater thermal hyperalgesia compared to wild-type mice after CFA injection and after nerve injury (but before PAP injection). See FIGS. 27C and 27E. Following i.t. injection of hPAP, thermal and mechanical thresholds increased in the inflamed/injured paws of wild-type mice but not in A₁R^{-/-} mice. See FIGS. 27C-27F. Likewise, the selective A₁R antagonist 8-cyclopentyl-1,3-dipropylxanthine (CPX; Sigma, St. Louis, Mo., United States of America; Catalog number C101; 1 mg/kg, i.p., dissolved in 0.9% saline containing 5% dimethylsulfoxide (DMSO), 1.25% NaOH) transiently reversed the anti-nociceptive effects of hPAP in control and inflamed hindpaws. See FIG. 29. Conversely, injection (i.t.) of the selective A₁R agonist N⁶-cyclopentyladenosine (CPA; Sigma, St. Louis, Mo., United States of America, Catalog number C8031; 10 mM stock solution in DMSO diluted in 0.9% saline) into wild-type mice produced dose-dependent increases in paw withdrawal latency to our thermal stimulus (see FIG. 30), similar to i.t. hPAP. However unlike hPAP, CPA had short-term effects (lasting hours not days) and CPA caused transient paralysis at the two highest doses. When taken together, these results demonstrate that the anti-nociceptive effects of PAP can be due to generation of adenosine followed by activation of A₁Rs. Moreover, these results suggest a novel in vivo function for PAP as an ectonucleotidase.

### Example 14

### High-Throughput Screen to Identify Small-Molecule Modulators of PAP

A high-throughput biochemical assay was developed to identify drugs that modulate PAP activity. This assay relies on the use of pure hPAP protein as well as a fluorometric PAP substrate (difluoro-4-methylumbelliferyl phosphate (DiFMUP); commercially available from Invitrogen). Dephosphorylation of DiFMUP by hPAP was monitored using fluorometric microplate readers (such as FLIPR or Flexstation). First, appropriate concentrations of hPAP protein and DiFMUP substrate were identified for use in 96-well plates, then 2,000 compounds (NCI Diversity Set) were screened to identify small-molecules that enhanced (activators) or suppressed (inhibitors) hPAP reaction rate. Using data from this screen, a Z-factor was calculated of 0.86 (this figure can range from 0-1; with 0.5 being the cutoff for a useful HTS. 0.86 is a very high value and indicates the assay is highly reproducible and has a large signal-to-noise ratio) (Zhang et al., 1999). From the screen, 6 candidate hPAP inhibitors and 3 candidate hPAP activators were identified. Fresh compounds (ordered from NCI) were obtained and dose-response experiments performed. These experiments confirmed that all 9 candidates were in fact activators or inhibitors. The extent to which these compounds were specific for hPAP was assessed by testing the effects of these compounds on hPAP, bPAP, potato acid phosphatase and bovine alkaline phosphatase. Thus, activators and inhibitors of hPAP can be identified using a reproducible, miniaturized, and economical HTS. The assay is useful to identify additional small molecule modulators of PAP.

### REFERENCES

The references listed below as well as all references cited in the specification, including patents, patent applications, journal articles, and all database entries (e.g., GENBANK® Accession Nos., including any annotations presented in the GENBANK® database that are associated with the disclosed sequences), are incorporated herein by reference to the extent that they supplement, explain, provide a background for, or teach methodology, techniques, and/or compositions employed herein.

Abdi, S., Lee, D. H., and Chung, J. M. (1998). The anti-allodynic effects of amitriptyline, gabapentin, and lidocaine in a rat model of neuropathic pain. Anesth Analg 87, 1360-1366.

Alderton, F., Darroch, P., Sambi, B., McKie, A., Ahmed, I. S., Pyne, N., and Pyne, S. (2001). G-protein-coupled receptor stimulation of the p42/p44 mitogen-activated protein kinase pathway is attenuated by lipid phosphate phosphatases 1, 1a, and 2 in human embryonic kidney 293 cells. J Biol Chem 276, 13452-13460.

Anliker, B., and Chun, J. (2004). Cell surface receptors in lysophospholipid signaling. Semin Cell Dev Biol 15, 457-465.

Baillie, J. K., and Power, I. (2006). The mechanism of action of gabapentin in neuropathic pain. Curr Opin Investig Drugs 7, 33-39.

Bandell, M., Story, G. M., Hwang, S. W., Viswanath, V., Eid, S. R., Petrus, M. J., Earley, T. J., and Patapoutian, A. (2004). Noxious cold ion channel TRPA1 is activated by pungent compounds and bradykinin. Neuron 41, 849-857.

Bandoh, K., Aoki, J., Hosono, H., Kobayashi, S., Kobayashi, T., Murakami-Murofushi, K., Tsujimoto, M., Arai, H., and Inoue, K. (1999). Molecular cloning and characterization of a novel human G-protein-coupled receptor, EDG7, for lysophosphatidic acid. J Biol Chem 274, 27776-27785.

Beers, S. A., Schwender, C. F., Loughney, D. A., Malloy, E., Demarest, K., and Jordan, J. (1996).

Phosphatase inhibitors—III. Benzylaminophosphonic acids as potent inhibitors of human prostatic acid phosphatase. Bioorg Med Chem 4, 1693-1701.

Bennett, G. J., and Xie, Y. K. (1988). A peripheral mononeuropathy in rat that produces disorders of pain sensation like those seen in man. Pain 33, 87-107.

Bockaert, J., and Pin, J. P. (1999). Molecular tinkering of G protein-coupled receptors: an evolutionary success. Embo J 18, 1723-1729.

Braz, J. M., Nassar, M. A., Wood, J. N., and Basbaum, A. I. (2005). Parallel “pain” pathways arise from subpopulations of primary afferent nociceptor. Neuron 47, 787-793.

Brindley, D. N., English, D., Pilquil, C., Buri, K., and Ling, Z. C. (2002). Lipid phosphate phosphatases regulate signal transduction through glycerolipids and sphingolipids. Biochim Biophys Acta 1582, 33-44.

Camargo, F., Erickson, R. P., Garver, W. S., Hossain, G. S., Carbone, P. N., Heidenreich, R. A., and Blanchard, J. (2001). Cyclodextrins in the treatment of a mouse model of Niemann-Pick C disease. Life Sci 70, 131-142.

Campbell, J. N., and Meyer, R. A. (2006). Mechanisms of neuropathic pain. Neuron 52, 77-92.

Carr, P. A., Yamamoto, T., and Nagy, J. I. (1990). Calcitonin gene-related peptide in primary afferent neurons of rat: co-existence with fluoride-resistant acid phosphatase and depletion by neonatal capsaicin. Neuroscience 36, 751-760.

Caterina, M. J., Leffler, A., Malmberg, A. B., Martin, W. J., Trafton, J., Petersen-Zeitz, K. R., Koltzenburg, M., Basbaum, A. I., and Julius, D. (2000). Impaired nociception and pain sensation in mice lacking the capsaicin receptor. Science 288, 306-313.

Chaplan, S. R., Bach, F. W., Pogrel, J. W., Chung, J. M., and Yaksh, T. L. (1994). Quantitative assessment of tactile allodynia in the rat paw. J Neurosci Methods 53, 55-63.

Chuang, H. H., Prescott, E. D., Kong, H., Shields, S., Jordt, S. E., Basbaum, A. I., Chao, M. V., and Julius, D. (2001). Bradykinin and nerve growth factor release the capsaicin receptor from Ptdlns(4,5)P2-mediated inhibition. Nature 411, 957-962.

Chung, J. M., and Chung, K. (2002). Importance of hyperexcitability of DRG neurons in neuropathic pain. Pain Pract 2, 87-97.

Cockayne, D. A., Hamilton, S. G., Zhu, Q. M., Dunn, P. M., Zhong, Y., Novakovic, S., Malmberg, A. B., Cain, G., Berson, A., Kassotakis, L., et al. (2000). Urinary bladder hyporeflexia and reduced pain-related behaviour in P2X3-deficient mice. Nature 407, 1011-1015.

Coimbra, A., Magalhaes, M. M., and Sodre-Borges, B. P. (1970). Ultrastructural localization of acid phosphatase in synaptic terminals of the rat substantia gelatinosa Rolandi. Brain Res 22, 142-146.

Colmant, H. J. (1959). Aktivitatsschwankungen der sauren Phosphatase im Ruckenmark and den Spinalganglien der Ratte nach Durchschneidung des N. ischiadicus. Arch Psychiat Nervnekr 199, 60-71.

Costigan, M., Befort, K., Karchewski, L., Griffin, R. S., D'Urso, D., Allchorne, A., Sitarski, J., Mannion, J. W., Pratt, R. E., and Woolf, C. J. (2002). Replicate high-density rat genome oligonucleotide microarrays reveal hundreds of regulated genes in the dorsal root ganglion after peripheral nerve injury. BMC Neurosci 3, 16.

Cox, J. J., Reimann, F., Nicholas, A. K., Thornton, G., Roberts, E., Springell, K., Karbani, G., Jafri, H., Mannan, J., Raashid, Y., et al. (2006). An SCN9A channelopathy causes congenital inability to experience pain. Nature 444, 894-898.

Craft, R. M., Mogil, J. S., and Aloisi, A. M. (2004). Sex differences in pain and analgesia: the role of gonadal hormones. Eur J Pain 8, 397-411.

Craig, A. D. (2003). Pain Mechanisms: Labeled lines verses convergence in central processing. Annu Rev Neurosci 26, 1-30.

Csillik, B., and Knyihar-Csillik, E. (1986). The Protean Gate: Structure and plasticity of the primary nociceptive analyzer (Budapest, Akademiai Kiado).

Cunha, T. M., Verri, W. A., Jr., Vivancos, G. G., Moreira, I. F., Reis, S., Parada, C. A., Cunha, F. Q., and Ferreira, S. H. (2004). An electronic pressure-meter nociception paw test for mice. Braz J Med Biol Res 37, 401-407.

Davar, G., Hama, A., Deykin, A., Vos, B., and Maciewicz, R. (1991). MK-801 blocks the development of thermal hyperalgesia in a rat model of experimental painful neuropathy. Brain Res 553, 327-330.

Davis-Taber, R. A. (2006). Transcriptional profiling of dorsal root ganglia in a neuropathic pain model using microarray and laser capture microdissection. Drug Dev Res 67, 308-330.

Decosterd, I., and Woolf, C. J. (2000). Spared nerve injury: an animal model of persistent peripheral neuropathic pain. Pain 87, 149-158.

Dirig, D. M., and Yaksh, T. L. (1995). Differential right shifts in the dose-response curve for intrathecal morphine and sufentanil as a function of stimulus intensity. Pain 62, 321-328.

Dodd, J., Jahr, C. E., Hamilton, P. N., Heath, M. J., Matthew, W. D., and Jessell, T. M. (1983). Cytochemical and physiological properties of sensory and dorsal horn neurons that transmit cutaneous sensation. Cold Spring Harb Symp Quant Biol 48 Pt 2, 685-695.

Dong, X., Han, S., Zylka, M. J., Simon, M. I., and Anderson, D. J. (2001). A diverse family of GPCRs expressed in specific subsets of nociceptive sensory neurons. Cell 106, 619-632.

Durgam, G. G., Tsukahara, R., Makarova, N., Walker, M. D., Fujiwara, Y., Pigg, K. R., Baker, D. L., Sardar, V. M., Parrill, A. L., Tigyi, G., and Miller, D. D. (2006). Synthesis and pharmacological evaluation of second generation phosphatidic acid derivatives as lysophosphatidic acid receptor ligands. Bioorg Med Chem Lett 16, 633-640.

Dworkin, R. H., Corbin, A. E., Young, J. P., Jr., Sharma, U., LaMoreaux, L., Bockbrader, H., Garofalo, E. A., and Poole, R. M. (2003). Pregabalin for the treatment of postherpetic neuralgia: a randomized, placebo controlled trial. Neurology 60, 1274-1283.

Dziembor-Gryszkiewicz, E., Fikus, M., Kazimierczuk, Z., and Ostrowski, W. (1978). Activity of human prostatic acid phosphatase toward purine 5′-phosphonucleosides. Bull Acad Pol Sci Biol 26, 815-821.

Eichholtz, T., Jalink, K., Fahrenfort, I., and Moolenaar, W. H. (1993). The bioactive phospholipid lysophosphatidic acid is released from activated platelets. Biochem J 291 (Pt 3), 677-680.

Elmes, S. J., Millns, P. J., Smart, D., Kendall, D. A., and Chapman, V. (2004). Evidence for biological effects of exogenous LPA on rat primary afferent and spinal cord neurons. Brain Res 1022, 205-213.

Fairbanks, C. A. (2003). Spinal delivery of analgesics in experimental models of pain and analgesia. Adv Drug Deliv Rev 55, 1007-1041.

Field, M. J., Cox, P. J., Stott, E., Melrose, H., Offord, J., Su, T. Z., Bramwell, S., Corradini, L., England, S., Winks, J., et al. (2006). Identification of the {alpha}2-{delta}-1 subunit of voltage-dependent calcium channels as a molecular target for pain mediating the analgesic actions of pregabalin. Proc Natl Acad Sci USA 103, 17537-17542.

Fleming, J., Ginn, S. L., Weinberger, R. P., Trahair, T. N., Smythe, J. A., and Alexander, I. E. (2001). Adenoassociated virus and lentivirus vectors mediate efficient and sustained transduction of cultured mouse and human dorsal root ganglia sensory neurons. Hum Gene Ther 12, 77-86.

Fujita, R., Kiguchi, N., and Ueda, H. (2006). LPA-mediated demyelination in ex vivo culture of dorsal root. Neurochem Int.

Fundin, B. T., Arvidsson, J., Aldskogius, H., Johansson, O., Rice, S. N., and Rice, F. L. (1997a). Comprehensive immunofluorescence and lectin binding analysis of intervibrissal fur innervation in the mystacial pad of the rat. J Comp Neurol 385, 185-206.

Fundin, B. T., Pfaller, K., and Rice, F. L. (1997b). Different distributions of the sensory and autonomic innervation among the microvasculature of the rat mystacial pad. J Comp Neurol 389, 545-568.

GENBANK® Accession Nos. NM_{—}001099; NP_{—}001090; NM_{—}001134194; NP_{—}001127666; NM_{—}207668; NP_{—}997551; NM_{—}019807; NP_{—}062781; NM_{—}001098866; NP_{—}001092336; NM_{—}001134901; NP_{—}001128373; NM_{—}020072; NP_{—}064457.

Giles, B. E., and Walker, J. S. (1999). Gender differences in pain. Curr Opin Anaesthesiol 12, 591-595.

Grant, G. J., Cascio, M., Zakowski, M. I., Langerman, L., and Turndorf, H. (1995). Intrathecal administration of liposomal morphine in a mouse model. Anesth Analg 81, 514-518.

Gutman, E. B., Sproul, E. E., and Gutman, A. B. (1936). Significance of increased phosphatase activity of one at the siteof osteoplastic metastases secondary to carcinoma of the prostate gland. Am J Cancer 28, 485-495.

Hains, B. C., and Waxman, S. G. (2006). Activated microglia contribute to the maintenance of chronic pain after spinal cord injury. J Neurosci 26, 4308-4317.

Han, S. K., Mancino, V., and Simon, M. I. (2006). Phospholipase cbeta 3 mediates the scratching response activated by the histamine h1 receptor on C-fiber nociceptive neurons. Neuron 52, 691-703.

Hargreaves, K., Dubner, R., Brown, F., Flores, C., and Joris, J. (1988). A new and sensitive method for measuring thermal nociception in cutaneous hyperalgesia. Pain 32, 77-88.

Hashimoto, T., Ohata, H., and Honda, K. (2006). Lysophosphatidic acid (LPA) induces plasma exudation and histamine release in mice via LPA receptors. J Pharmacol Sci 100, 82-87.

Hashimoto, T., Ohata, H., and Momose, K. (2004). Itch-scratch responses induced by lysophosphatidic acid in mice. Pharmacology 72, 51-56.

Heasley, B. H., Jarosz, R., Carter, K. M., Van, S. J., Lynch, K. R., and Macdonald, T. L. (2004). A novel series of 2-pyridyl-containing compounds as lysophosphatidic acid receptor antagonists: development of a nonhydrolyzable LPA3 receptor-selective antagonist. Bioorg Med Chem Lett 14, 4069-4074.

Hiroyama, M., and Takenawa, T. (1999). Isolation of a cDNA encoding human lysophosphatidic acid phosphatase that is involved in the regulation of mitochondrial lipid biosynthesis. J Biol Chem 274, 29172-29180.

Hua, X., Erikson, C. J., Chason, K. D., Rosebrock, C. N., Deshpande, D. A., Penn, R. B., and Tilley, S. L. (2007). Involvement of A1 adenosine receptors and neural pathways in adenosine-induced bronchoconstriction in mice. Am J Physiol Lung Cell Mol Physiol 293, L25-32.

Hunt, S. P., and Mantyh, P. W. (2001). The molecular dynamics of pain control. Nat Rev Neurosci 2, 83-91.

Hunt, S. P., and Rossi, J. (1985). Peptide- and non-peptide-containing unmyelinated primary afferents: the parallel processing of nociceptive information. Philos Trans R Soc Lond B Biol Sci 308, 283-289.

Hylden, J. L., and Wilcox, G. L. (1980). Intrathecal morphine in mice: a new technique. Eur J Pharmacol 67, 313-316.

Inoue, M., Rashid, M. H., Fujita, R., Contos, J. J., Chun, J., and Ueda, H. (2004). Initiation of neuropathic pain requires lysophosphatidic acid receptor signaling. Nat Med 10, 712-718.

Ishii, I., Contos, J. J., Fukushima, N., and Chun, J. (2000). Functional comparisons of the lysophosphatidic acid receptors, LP(A1)/VZG-1/EDG-2, LP(A2)/EDG-4, and LP(A3)/EDG-7 in neuronal cell lines using a retrovirus expression system. Mol Pharmacol 58, 895-902.

Ji, R. R., and Strichartz, G. (2004). Cell signaling and the genesis of neuropathic pain. Sci STKE 2004, reE14.

Johansson, B., Halldner, L., Dunwiddie, T. V., Masino, S. A., Poelchen, W., Gimenez-Llort, L., Escorihuela, R. M., Fernandez-Teruel, A., Wiesenfeld-Hallin, Z., Xu, X. J., et al. (2001). Hyperalgesia, anxiety, and decreased hypoxic neuroprotection in mice lacking the adenosine A1 receptor. Proc Natl Acad Sci USA 98, 9407-9412.

Jordt, S. E., McKemy, D. D., and Julius, D. (2003). Lessons from peppers and peppermint: the molecular logic of thermosensation. Curr Opin Neurobiol 13, 487-492.

Julius, D., and Basbaum, A. I. (2001). Molecular mechanisms of nociception. Nature 413, 203-210.

Kaija, H., Patrikainen, L. O. T., Alatalo, S. L., Vaananen, H. K. & Vihko, P. T. Acid Phosphatases. in Dynamics of Bone and Cartilage Metabolism (eds. Seibel, M. J., Robins, S. P. & Bilezikian, J. P.) 165-180 (Academic Press, 2006).

Keeley, M. B., Busch, J., Singh, R., and Abel, T. (2005). TetR hybrid transcription factors report cell signaling and are inhibited by doxycycline. Biotechniques 39, 529-536.

Kim, S. H., and Chung, J. M. (1992). An experimental model for peripheral neuropathy produced by segmental spinal nerve ligation in the rat. Pain 50, 355-363.

Knyihar-Csillik, E., Bezzegh, A., Boti, S., and Csillik, B. (1986). Thiamine monophosphatase: a genuine marker for transganglionic regulation of primary sensory neurons. J Histochem Cytochem 34, 363-371.

Knyihar, E., and Gerebtzoff, M. A. (1970). Localisation ultrastructurale de I' isoenzyme fluororesistant de Ia phosphatase acide dans Ia moelle epiniere de rat. Bull Assoc Anat 149, 768-790.

Kranenburg, O., Poland, M., van Horck, F. P., Drechsel, D., Hall, A., and Moolenaar, W. H. (1999). Activation of RhoA by lysophosphatidic acid and Galpha12/13 subunits in neuronal cells: induction of neurite retraction. Mol Biol Cell 10, 1851-1857.

Kume, K., Zylka, M. J., Sriram, S., Shearman, L. P., Weaver, D. R., Jin, X., Canwood, E. S., Hastings, M. H., and. Reppert, S. M. (1999). mCRY1 and mCRY2 are essential components of the negative limb of the circadian clock feedback loop. Cell 98, 193-205.

LaBuda, C. J., and Little, P. J. (2005). Pharmacological evaluation of the selective spinal nerve ligation model of neuropathic pain in the rat. J Neurosci Methods 144, 175-181.

Lazarowski, E. R., Tarran, R., Grubb, B. R., van Heusden, C. A., Okada, S., and Boucher, R. C. (2004). Nucleotide release provides a mechanism for airway surface liquid homeostasis. J Biol Chem 279, 36855-36864.

Lee, C. W., Rivera, R., Gardell, S., Dubin, A. E., and Chun, J. (2006). GPR92 as a new G12/13- and Gq coupled lysophosphatidic acid receptor that increases cAMP, LPA5. J Biol Chem 281, 23589-23597.

Lee, W. S., Hong, M. P., Hoon Kim, T., Kyoo Shin, Y., Soo Lee, C., Park, M., and Song, J. H. (2005). Effects of lysophosphatidic acid on sodium currents in rat dorsal root ganglion neurons. Brain Res 1035, 100-104.

Lee, Y. W., and Yaksh, T. L. (1996). Pharmacology of the spinal adenosine receptor which mediates the antiallodynic action of intrathecal adenosine agonists. J Pharmacol Exp Ther 277, 1642-1648.

Lei, L., Laub, F., Lush, M., Romero, M., Zhou, J., Luikart, B., Klesse, L., Ramirez, F., and Parada, L. F. (2005). The zinc finger transcription factor Klf7 is required for TrkA gene expression and development of nociceptive sensory neurons. Genes Dev 19, 1354-1364.

Li, J., and Perl, E. R. (1995). ATP modulation of synaptic transmission in the spinal substantia gelatinosa. J Neurosci 15, 3357-3365.

Lin, M. F., Garcia-Arenas, R., Xia, X. Z., Biela, B., and Lin, F. F. (1994). The cellular level of prostatic acid phosphatase and the growth of human prostate carcinoma cells. Differentiation 57, 143-149.

Liu, X. J., and Salter, M. W. (2005). Purines and pain mechanisms: recent developments. Curr Opin Investig Drugs 6, 65-75.

Lu, Y., and Perl, E. R. (2003). A specific inhibitory pathway between substantia gelatinosa neurons receiving direct C-fiber input. J Neurosci 23, 8752-8758.

Luo, Z. D., Chaplan, S. R., Higuera, E. S., Sorkin, L. S., Stauderman, K. A., Williams, M. E., and Yaksh, T. L. (2001). Upregulation of dorsal root ganglion (alpha)2(delta) calcium channel subunit and its correlation with allodynia in spinal nerve-injured rats. J Neurosci 21, 1868-1875.

Lynch, M. E., Clark, A. J., and Sawynok, J. (2003). Intravenous adenosine alleviates neuropathic pain: a double blind placebo controlled crossover trial using an enriched enrolment design. Pain 103, 111-117.

Malmberg, A. B., and Basbaum, A. I. (1998). Partial sciatic nerve injury in the mouse as a model of neuropathic pain: behavioral and neuroanatomical correlates. Pain 76, 215-222.

Malmberg, A. B., Chen, C., Tonegawa, S., and Basbaum, A. I. (1997). Preserved acute pain and reduced neuropathic pain in mice lacking PKCgamma. Science 278, 279-283.

Maneuf, Y. P., Luo, Z. D., and Lee, K. (2006). alpha2delta and the mechanism of action of gabapentin in the treatment of pain. Semin Cell Dev Biol.

McMahon, S. B. (1986). The localization of fluoride-resistant acid phosphatase (FRAP) in the pelvic nerves and sacral spinal cord of rats. Neurosci Lett 64, 305-310.

McMahon, S. B., and Moore, C. E. (1988). Plasticity of primary afferent acid phosphatase expression following rerouting of afferents from muscle to skin in the adult rat. J Comp Neurol 274, 1-8.

McTigue, J. J., and Van Etten, R. L. (1978). An essential active-site histidine residue in human prostatic acid phosphatase. Ethoxyformylation by diethyl pyrocarbonate and phosphorylation by a substrate. Biochim Biophys Acta 523, 407-421.

Mills, G. B., and Moolenaar, W. H. (2003). The emerging role of lysophosphatidic acid in cancer. Nat Rev Cancer 3, 582-591.

Moller, T., Contos, J. J., Musante, D. B., Chun, J., and Ransom, B. R. (2001). Expression and function of lysophosphatidic acid receptors in cultured rodent microglial cells. J Biol Chem 276, 25946-25952.

Moolenaar, W. H., van Meeteren, L. A., and Giepmans, B. N. (2004). The ins and outs of lysophosphatidic acid signaling. Bioessays 26, 870-881.

Moqrich, A., Earley, T. J., Watson, J., Andahazy, M., Backus, C., Martin-Zanca, D., Wright, D. E., Reichardt, L. F., and Patapoutian, A. (2004). Expressing TrkC from the TrkA locus causes a subset of dorsal root ganglia neurons to switch fate. Nat Neurosci 7, 812-818.

Mulder, H., Zhang, Y., Danielsen, N., and Sundler, F. (1997). Islet amyloid polypeptide and calcitonin generelated peptide expression are down-regulated in dorsal root ganglia upon sciatic nerve transection. Brain Res Mol Brain Res 47, 322-330.

Nagai, T., Ibata, K., Park, E. S., Kubota, M., Mikoshiba, K., and Miyawaki, A. (2002). A variant of yellow fluorescent protein with fast and efficient maturation for cell-biological applications. Nat Biotechnol 20, 87-90.

Nagy, J. I., and Hunt, S. P. (1982). Fluoride-resistant acid phosphatase-containing neurones in dorsal root ganglia are separate from those containing substance P or somatostatin. Neuroscience 7, 89-97.

Nishiyama, T., Ho, R. J., Shen, D. D., and Yaksh, T. L. (2000). The effects of intrathecal morphine encapsulated in L- and D-dipalmitoylphosphatidyl choline liposomes on acute nociception in rats. Anesth Analg 91, 423-428.

Noguchi, K., Ishii, S., and Shimizu, T. (2003). Identification of p2y9/GPR23 as a novel G protein-coupled receptor for lysophosphatidic acid, structurally distant from the Edg family. J Biol Chem 278, 25600-25606.

Ostrowski, W. S., and Kuciel, R. (1994). Human prostatic acid phosphatase: selected properties and practical applications. Clin Chim Acta 226, 121-129.

Park, K. A., and Vasko, M. R. (2005). Lipid mediators of sensitivity in sensory neurons. Trends Pharmacol Sci 26, 571-577.

Patapoutian, A., Peier, A. M., Story, G. M., and Viswanath, V. (2003). ThermoTRP channels and beyond: mechanisms of temperature sensation. Nat Rev Neurosci 4, 529-539.

Patterson, S. L., Sluka, K. A., and Arnold, M. A. (2001). A novel transverse push-pull microprobe: in vitro characterization and in vivo demonstration of the enzymatic production of adenosine in the spinal cord dorsal horn. J Neurochem 76, 234-246.

Pilquil, C., Singh, I., Zhang, Q., Ling, Z., Buri, K., Stromberg, L. M., Dewald, J., and Brindley, D. N. (2001). Lipid phosphate phosphatase-1 dephosphorylates exogenous lysophosphatidate and thereby attenuates its effects on cell signalling. Prostaglandins 64, 83-92.

Piros, E. T., Hales, T. G., and Evans, C. J. (1996). Functional analysis of cloned opioid receptors in transfected cell lines. Neurochem Res 21, 1277-1285.

Porvari, K., Kurkela, R., Kivinen, A., and Vihko, P. (1995). Differential androgen regulation of rat prostatic acid phosphatase transcripts. Biochem Biophys Res Commun 213, 861-868.

Post, C. (1984). Antinociceptive effects in mice after intrathecal injection of 5′-N-ethylcarboxamide adenosine. Neurosci Lett 51, 325-330.

Pyne, S., Long, J. S., Ktistakis, N. T., and Pyne, N. J. (2005). Lipid phosphate phosphatases and lipid phosphate signalling. Biochem Soc Trans 33, 1370-1374.

Quintero, Ileana B., Ce'sar L. Araujo, Anitta E. Pulkka, Riikka S. Wirkkala, Annakaisa M. Herrala, Eeva-Liisa Eskelinen, Eija Jokitalo, Pekka A. Hellstro\ddot{}m, Hannu J. Tuominen, Pasi P. Hirvikoski, and Pirkko T. Vihko (2007). Prostatic Acid Phosphatase Is Not a Prostate Specific Target. Cancer Res 67(14), 6549-6554.

Renback, K., Inoue, M., and Ueda, H. (1999). Lysophosphatidic acid-induced, pertussis toxin-sensitive nociception through a substance P release from peripheral nerve endings in mice. Neurosci Lett 270, 59-61.

Renback, K., Inoue, M., Yoshida, A., Nyberg, F., and Ueda, H. (2000). Vzg-1/lysophosphatidic acid-receptor involved in peripheral pain transmission. Brain Res Mol Brain Res 75, 350-354.

Rice, F. L. (1993). Structure, vascularization, and innervation of the mystacial pad of the rat as revealed by the lectin Griffonia simplicifolia. J Comp Neurol 337, 386-399.

Rice, F. L., Kinnman, E., Aldskogius, H., Johansson, O., and Arvidsson, J. (1993). The innervation of the mystacial pad of the rat as revealed by PGP 9.5 immunofluorescence. J Comp Neurol 337, 366-385.

Rocheville, M., Lange, D. C., Kumar, U., Patel, S. C., Patel, R. C., and Patel, Y. C. (2000). Receptors for dopamine and somatostatin: formation of hetero-oligomers with enhanced functional activity. Science 288, 154-157.

Roiko, K., Janne, O. A., and Vihko, P. (1990). Primary structure of rat secretory acid phosphatase and comparison to other acid phosphatases. Gene 89, 223-229.

Sanyal, S., and Rustioni, A. (1974). Phosphatases in the substantia gelatinosa and motoneurones: a comparative histochemical study. Brain Res 76, 161-166.

Sawynok, J. (2006). Adenosine and ATP receptors. Handb Exp Pharmacol 177, 309-328.

Schilling, T., Stock, C., Schwab, A., and Eder, C. (2004). Functional importance of Ca2+-activated K+ channels for lysophosphatidic acid-induced microglial migration. Eur J Neurosci 19, 1469-1474.

Schmelz, M., Schmidt, R., Bickel, A., Handwerker, H. O., and Torebjork, H. E. (1997). Specific C-receptors for itch in human skin. J Neurosci 17, 8003-8008.

Schneider, G., Lindqvist, Y., and Vihko, P. (1993). Three-dimensional structure of rat acid phosphatase. Embo J 12, 2609-2615.

Seltzer, Z., Dubner, R., and Shir, Y. (1990). A novel behavioral model of neuropathic pain disorders produced in rats by partial sciatic nerve injury. Pain 43, 205-218.

Shaner, N. C., Campbell, R. E., Steinbach, P. A., Giepmans, B. N., Palmer, A. E., and Tsien, R. Y. (2004). Improved monomeric red, orange and yellow fluorescent proteins derived from Discosoma sp. red fluorescent protein. Nat Biotechnol 22, 1567-1572.

Shi, T. J., Tandrup, T., Bergman, E., Xu, Z. Q., Ulfhake, B., and Hokfelt, T. (2001). Effect of peripheral nerve injury on dorsal root ganglion neurons in the C57 BL/6J mouse: marked changes both in cell numbers and neuropeptide expression. Neuroscience 105, 249-263.

Shields, S. D., Eckert, W. A., 3rd, and Basbaum, A. I. (2003). Spared nerve injury model of neuropathic pain in the mouse: a behavioral and anatomic analysis. J Pain 4, 465-470.

Shimizu, I., lida, T., Guan, Y., Zhao, C., Raja, S. N., Jarvis, M. F., Cockayne, D. A., and Caterina, M. J. (2005). Enhanced thermal avoidance in mice lacking the ATP receptor P2X3. Pain 116, 96-108.

Silverman, J. D., and Kruger, L. (1988a). Acid phosphatase as a selective marker for a class of small sensory ganglion cells in several mammals: spinal cord distribution, histochemical properties, and relation to fluorideresistant acid phosphatase (FRAP) of rodents. Somatosens Res 5, 219-246.

Silverman, J. D., and Kruger, L. (1988b). Lectin and neuropeptide labeling of separate populations of dorsal root ganglion neurons and associated “nociceptor” thin axons in rat testis and cornea whole-mount preparations. Somatosens Res 5, 259-267.

Silverman, J. D., and Kruger, L. (1990). Selective neuronal glycoconjugate expression in sensory and autonomic ganglia: relation of lectin reactivity to peptide and enzyme markers. J Neurocytol 19, 789-801.

Sjolund, K. F., Belfrage, M., Karlsten, R., Segerdahl, M., Arner, S., Gordh, T., and Solevi, A. (2001). Systemic adenosine infusion reduces the area of tactile allodynia in neuropathic pain following peripheral nerve injury: a multi-centre, placebo-controlled study. Eur J Pain 5, 199-207.

Snider, W. D., and McMahon, S. B. (1998). Tackling pain at the source: new ideas about nociceptors. Neuron 20, 629-632.

Souslova, V., Cesare, P., Ding, Y., Akopian, A. N., Stanfa, L., Suzuki, R., Carpenter, K., Dickenson, A., Boyce, S., Hill, R., et al. (2000). Warm-coding deficits and aberrant inflammatory pain in mice lacking P2X3 receptors. Nature 407, 1015-1017.

Stucky, C. L., Koltzenburg, M., Schneider, M., Engle, M. G., Albers, K. M., and Davis, B. M. (1999). Overexpression of nerve growth factor in skin selectively affects the survival and functional properties of nociceptors. J Neurosci 19, 8509-8516.

Sugiura, T., Nakane, S., Kishimoto, S., Waku, K., Yoshioka, Y., Tokumura, A., and Hanahan, D. J. (1999). Occurrence of lysophosphatidic acid and its alkyl ether-linked analog in rat brain and comparison of their biological activities toward cultured neural cells. Biochim Biophys Acta 1440, 194-204.

Takuwa, Y., Takuwa, N., and Sugimoto, N. (2002). The Edg family G protein-coupled receptors for lysophospholipids: their signaling properties and biological activities. J Biochem (Tokyo) 131, 767-771.

Tanaka, M., Kishi, Y., Takanezawa, Y., Kakehi, Y., Aoki, J., and Arai, H. (2004). Prostatic acid phosphatase degrades lysophosphatidic acid in seminal plasma. FEBS Lett 571, 197-204.

Tegeder, I., Costigan, M., Griffin, R. S., Abele, A., Belfer, I., Schmidt, H., Ehnert, C., Nejim, J., Marian, C., Scholz, J., et al. (2006). GTP cyclohydrolase and tetrahydrobiopterin regulate pain sensitivity and persistence. Nat Med.

Tenser, R. B. (1985). Sequential changes of sensory neuron (fluoride-resistant) acid phosphatase in dorsal root ganglion neurons following neurectomy and rhizotomy. Brain Res 332, 386-389.

Tenser, R. B., Viselli, A. L., and Savage, D. H. (1991). Reversible decrease of fluoride resistant acid phosphatase-positive neurons after herpes simplex virus infection. Neurosci Lett 130, 85-88.

Tigyi, G., Dyer, D. L., and Miledi, R. (1994). Lysophosphatidic acid possesses dual action in cell proliferation. Proc Natl Acad Sci USA 91, 1908-1912.

Tigyi, G., Fischer, D. J., Baker, D., Wang, D. A., Yue, J., Nusser, N., Virag, T., Zsiros, V., Liliom, K., Miller, D., and Parrill, A. (2000). Pharmacological characterization of phospholipid growth-factor receptors. Ann N Y Acad Sci 905, 34-53.

Tigyi, G., Fischer, D. J., Sebok, A., Yang, C., Dyer, D. L., and Miledi, R. (1996). Lysophosphatidic acid-induced neurite retraction in PC12 cells: control by phosphoinositide-Ca2+ signaling and Rho. J Neurochem 66, 537-548.

Tigyi, G., and Miledi, R. (1992). Lysophosphatidates bound to serum albumin activate membrane currents in Xenopus oocytes and neurite retraction in PC12 pheochromocytoma cells. J Biol Chem 267, 21360-21367.

Tsuda, M., Shigemoto-Mogami, Y., Koizumi, S., Mizokoshi, A., Kohsaka, S., Salter, M. W., and Inoue, K. (2003). P2X4 receptors induced in spinal microglia gate tactile allodynia after nerve injury. Nature 424, 778-783.

U.S. Pat. No. 6,972,174.

U.S. Pat. No. 6,110,709.

Vadakkan, K. I., Jia, Y. H., and Zhuo, M. (2005). A behavioral model of neuropathic pain induced by ligation of the common peroneal nerve in mice. J Pain 6, 747-756.

Van Brocklyn, J. R., Graler, M. H., Bernhardt, G., Hobson, J. P., Lipp, M., and Spiegel, S. (2000). Sphingosine-1-phosphate is a ligand for the G protein-coupled receptor EDG-6. Blood 95, 2624-2629.

van Koppen, C., Meyer zu Heringdorf, M., Laser, K. T., Zhang, C., Jakobs, K. H., Bunemann, M., and Pott, L. (1996). Activation of a high affinity Gi protein-coupled plasma membrane receptor by sphingosine-1-phosphate. J Biol Chem 271, 2082-2087.

Vihko, P., Kurkela, R., Porvari, K., Herrala, A., Lindfors, A., Lindqvist, Y., and Schneider, G. (1993). Rat acid phosphatase: overexpression of active, secreted enzyme by recombinant baculovirus-infected insect cells, molecular properties, and crystallization. Proc Nati Acad Sci USA 90, 799-803.

Vihko, P., Sajanti, E., Janne, O., Peltonen, L., and Vihko, R. (1978a). Serum prostate-specific acid phosphatase: development and validation of a specific radioimmunoassay. Clin Chem 24, 1915-1919.

Vihko, P. (1978b). Characterization of the principal human prostatic acid phosphatase isoenzyme, purified by affinity chromatography and isoelectric focusing. Part II. Clin Chem 24, 1783-1787.

Vihko, P., Virkkunen, P., Henttu, P., Roiko, K., Solin, T., and Huhtala, M. L. (1988). Molecular cloning and sequence analysis of cDNA encoding human prostatic acid phosphatase. FEBS Lett 236, 275-281.

Wang, H., Rivero-Melian, C., Robertson, B., and Grant, G. (1994). Transganglionic transport and binding of the isolectin B4 from Griffonia simplicifolia I in rat primary sensory neurons. Neuroscience 62, 539-551.

Wang, L., and Tsien, R. Y. (2006). Evolving proteins in mammalian cells using somatic hypermutation. Nat Protoc 1, 1346-1350.

Weiner, J. A., and Chun, J. (1999). Schwann cell survival mediated by the signaling phospholipid lysophosphatidic acid. Proc Nati Acad Sci USA 96, 5233-5238.

WO9302212.

WO0020853.

Woodbury, C. J., Ritter, A. M., and Koerber, H. R. (2000). On the problem of lamination in the superficial dorsal horn of mammals: a reappraisal of the substantia gelatinosa in postnatal life. J Comp Neurol 417, 88-102.

Wu, Li-Chen, Sun, Chiao-Wang, Ryan, Thomas M., Pawlik, Kevin M., Ren, Jinxiang, and Townes, Tim M. (2006). Correction of sickle cell disease by homologous recombination in embryonic stem cells. Blood 108(4), 1183-1188.

Xie, Y., Gibbs, T. C., Mukhin, Y. V., and Meier, K. E. (2002). Role for 18:1 lysophosphatidic acid as an autocrine mediator in prostate cancer cells. J Biol Chem 277, 32516-32526.

Ye, X., Hama, K., Contos, J. J., Anliker, B., Inoue, A., Skinner, M. K., Suzuki, H., Amano, T., Kennedy, G., Arai, H., et al. (2005). LPA3-mediated lysophosphatidic acid signalling in embryo implantation and spacing. Nature 435, 104-108.

Zhang, J. H., Chung, T. D., and Oldenburg, K. R. (1999). A Simple Statistical Parameter for Use in Evaluation and Validation of High Throughput Screening Assays. J Biomol Screen 4, 67-73.

Zhang, X., Davidson, S., and Giesler, G. J., Jr. (2006). Thermally identified subgroups of marginal zone neurons project to distinct regions of the ventral posterior lateral nucleus in rats. J Neurosci 26, 5215-5223.

Zhao, Y., Usatyuk, P. V., Cummings, R., Saatian, B., He, D., Watkins, T., Morris, A., Spannhake, E. W., Brindley, D. N., and Natarajan, V. (2005). Lipid phosphate phosphatase-1 regulates lysophosphatidic acidinduced calcium release, NF-kappaB activation and interleukin-8 secretion in human bronchial epithelial cells. Biochem J 385, 493-502.

Zwick, M., Davis, B. M., Woodbury, C. J., Burkett, J. N., Koerber, H. R., Simpson, J. F., and Albers, K. M. (2002). Glial cell line-derived neurotrophic factor is a survival factor for isolectin B4-positive, but not vanilloid receptor 1-positive, neurons in the mouse. J Neurosci 22, 4057-4065.

Zylka, M. J., Dong, X., Southwell, A. L., and Anderson, D. J. (2003). Atypical expansion in mice of the sensory neuron-specific Mrg G protein-coupled receptor family. Proc Natl Acad Sci USA 100, 10043-10048.

Zylka, M. J., Rice, F. L., and Anderson, D. J. (2005). Topographically distinct epidermal nociceptive circuits revealed by axonal tracers targeted to Mrgprd. Neuron 45, 17-25.

It will be understood that various details of the presently disclosed subject matter can be changed without departing from the scope of the presently disclosed subject matter. Furthermore, the foregoing description is for the purpose of illustration only, and not for the purpose of limitation.

What is claimed is:

1. A method for treating pain in an animal by administering a composition or a pharmaceutical formulation comprising a therapeutically effective amount of a PAP, or an active variant, fragment or derivative thereof, or a therapeutically effective amount of an activity enhancing PAP modulator.

2. The method of claim 1, wherein the pain is characterized by one or more of: chronic pain, chronic inflammatory pain, neuropathic pain, chronic neuropathic pain, allodynia, hyperalgesia, nerve injury, trauma, tissue injury, inflammation, cancer, viral infection, Shingles, diabetic neuropathy, osteoarthritis, burns, joint pain, lower back pain, visceral pain, migraine headache, cluster headache, headache, fibromyalgia or pain associated with childbirth.

3. A method of treating an animal for a disorder characterized at least in part by an excess of lysophosphatidic acid, comprising administering to the animal a composition or pharmaceutical formulation comprising a therapeutically effective amount of a PAP, or an active variant, fragment or derivative thereof, or a therapeutically effective amount of an activity enhancing PAP modulator.

4. A method of treating an animal for a disorder characterized at least in part by a deficiency in adenosine or adenosine receptor function, comprising administering to the animal a composition or pharmaceutical formulation comprising a therapeutically effective amount of a PAP, or an active variant, fragment or derivative thereof, or a therapeutically effective amount of an activity enhancing PAP modulator.

5. The method of claims 1, 3 or 4 wherein the animal is a human.

6. The method of claims 1, 3 or 4, wherein the PAP, is selected from the group consisting of human PAP, bovine PAP, rat PAP and mouse PAP, and active fragments, variants and derivatives thereof.

7. The method of claims 1, 3 or 4, wherein the PAP or the active variant, fragment or derivative thereof, comprises one or more modifications selected from the group consisting of one or more: conservative amino acid substitutions; non-natural amino acid substitutions, D- or D,L-racemic mixture isomer form amino acid substitutions, amino acid chemical substitutions, carboxy- or amino-terminus modifications, conjugation to biocompatible molecules including but not limited to fatty acids and PEG and conjugation to biocompatible support structures including but not limited to agarose, sepharose and nanoparticles.

8. The method of claims 1, 3 or 4, wherein the PAP is obtained by recombinant methods.

9. The method of claims 1, 3 or 4, wherein the PAP, or the active variant, fragment or derivative thereof, or the activity enhancing modulator of the PAP is administered via one or more of injection, oral administration, a surgically implanted pump, stem cells, viral gene therapy and naked DNA gene therapy.

10. The method of claim 9, wherein the administration is via intravenous injection, epideral injection, or intrathecal injection.

11. The method of claim 10, wherein the administration is via intrathecal injection of PAP-expressing embryonic stem cells.

12. The method of claim 10, wherein the administration is by intrathecal injection about once every 3 days.

13. The method of claim 9, wherein the administration is in combination with one or more of adenosine, adenosine monophosphate (AMP), an AMP analogue, an adenosine kinase inhibitor, 5′-amino-5′-deoxyadenosine, 5-iodotubercidin, an adenosine deaminase inhibitor, 2′-deoxycoformycin, a nucleoside transporter inhibitor, dipyridamole.

14. The method of claim 9, wherein the administration is in combination with one or more known analgesic.

15. The method of claim 14, wherein the known analgesic is an opiate.

16. The method of claim 9, wherein the administration is via viral gene therapy using a retroviral, adenoviral or adeno-associated viral vector transfer cassette comprising a nucleic acid sequence encoding the PAP or active variant or fragment thereof.

17. A method of treating cystic fibrosis in an animal, the method comprising administering to the animal a composition or pharmaceutical formulation comprising a therapeutically effective amount of a PAP, or an active variant, fragment or derivative thereof, or a therapeutically effective amount of an activity enhancing PAP modulator.

18. The method of claim 17, wherein the administering is by aerosolizing in the lungs.

19. A method of increasing levels of adenosine in the lungs of an animal having a disorder characterized at least in part by a deficiency in adenosine or adenosine receptor function, the method comprising administering to the animal a composition or pharmaceutical formulation comprising a therapeutically effective amount of a PAP, or an active variant, fragment or derivative thereof, or a therapeutically effective amount of an activity enhancing PAP modulator.

20. An isolated PAP peptide, the peptide selected from the group consisting of human PAP, cow PAP, rat PAP and mouse PAP, and active fragments, variants, and derivatives thereof.

21. An isolated nucleotide sequence encoding for the PAP peptide of claim 20.

22. An expression vector comprising the nucleotide sequence of claim 21.

23. A host cell comprising the expression vector of claim 22.

24. A composition comprising the peptide of claim 20, wherein the composition is prepared for administration to animals, or as a pharmaceutical formulation for administration to humans.

25. A method of screening for a small molecule modulator of PAP activity by measuring the activity of a PAP in the presence and absence of a candidate small molecule and identifying as PAP modulators the candidate small molecules that cause either an increase or a decrease in the PAP activity.

26. A kit for the treatment of pain in animals, comprising a composition or pharmaceutical formulation comprising a therapeutically effective amount of a PAP, or an active variant, fragment or derivative thereof, and a surgically implantable pump apparatus for delivery of PAP to local tissue.

27. A method for diagnosing an individual's response to a pain medicine, comprising identifying one or more single nucleotide polymorphisms (SNPs), insertions or deletions in and around a PAP genomic locus in the individual; and correlating the SNPs with a predetermined response to the pain medicine.

28. A method for diagnosing an individual's threshold for pain, comprising identifying one or more single nucleotide polymorphisms (SNPs) insertions or deletions in and around a PAP genomic locus in the individual; and correlating the SNPs with a predetermined threshold for pain.

29. A method for diagnosing an individual's propensity to transition from acute to chronic pain, comprising identifying one or more single nucleotide polymorphisms (SNPs) insertions or deletions in and around a PAP genomic locus in the individual; and correlating the SNPs with a predetermined threshold for pain.

30. A method for diagnosing an individual's response to a pain medicine, comprising correlating differences in PAP expression in the individual and a control population, and correlating the extent of differential expression with a predetermined response to the pain medication.

31. A method for diagnosing an individual's threshold for pain, comprising correlating differences in PAP expression in the individual and a control population, and correlating the extent of differential expression with a predetermined threshold for pain.

32. A method for diagnosing an individual's propensity to transition from acute to chronic pain, comprising correlating differences in PAP expression in the individual and a control population, and correlating the extent of differential expression with a propensity to transition from acute to chronic pain.


SEQUENCE LISTING (6 sequences)

SEQ ID NO: 1 - protein, 7 aa, Artificial
  artificially synthesized
RHGXRXP

SEQ ID NO: 2 - DNA, 1254 bp, Mus musculus
atgcgagccgttcctctgcccctgagccggacagcaagcctcagccttggcttcttgctcctgctttctctctgcctggacccaggccaagccaaggagttgaagtttgtgacattggtgtttcggcatggagaccgaggtcccatcgagacctttcctaccgaccccattacagaatcctcgtggccacaaggatttggccaactcacccagtggggcatggaacagcactacgaacttggaagttatataaggaaaagatacggaagattcttgaacgacacctataagcatgatcagatttatatccggagcacagatgtggacaggactttgatgagtgctatgacaaaccttgcagccctgtttcctccagaggggatcagcatctggaatcctagactgctctggcagcccatcccagtgcacaccgtgtctctctctgaggatcggttgctgtacctgcctttcagagactgccctcgttttgaagaactcaagagtgagactttagaatctgaggaattcttgaagaggcttcatccatataaaagcttcctggacaccttgtcgtcgctgtcgggattcgatgaccaggatctttttggaatctggagtaaagtttatgaccctttattctgcgagagtgttcacaatttcaccttgccctcctgggccaccgaggacgccatgattaagttgaaagagctatcagaattatctctgctatcactttatggaattcacaagcagaaagagaaatctcgactccaagggggcgtcctggtcaatgaaatcctcaagaatatgaagcttgcaactcagccacagaagtataaaaagctggtcatgtattccgcacacgacactaccgtgagtggcctgcagatggcgctagatgtttataatggagttctgcctccctacgcttcttgccacatgatggaattgtaccatgataagggggggcactttgtggagatgtactatcggaatgagacccagaacgagccctacccactcacgctgccaggctgcacccacagctgccctctggagaagtttgcggagctactggacccggtgatctcccaggactgggccacggagtgtatggccacaagcagccaccaagtgctgagggttatccttgccactacattttgcctggtaaccgggatcctggtgatacttctgcttgtcctcatccgccatgggccctgctggcagagagatgtgtatcggaacatctga

SEQ ID NO: 3 - DNA, 1082 bp, Mus musculus
catggcttccatacagcagctcctccaaggcgcgttgtagtcaataaacaacctgacaagagcaagatatcagacacagcacctcagaggtcagagtgctctgcagagggtctcccaagcctccagtgcctaacatgcagaatcaagagctaatacatagaggagctatgggggaggcacacaagcacacacacacgcacacacacacacacaccactgaaacaaaatgcctccaagtccaaaactaattggcactttgagtgtaggctacaaaatcctctcagagaacaaacaatgttcaaggctttgattctggaaccttcagaagccgtaagccaagtaccaggtctacagccactgcacacggagctgtgccccgatgatgctctttagagcctgactggccatgtggcagaacacgcctgggattctaaattggatagagacaagatggctgccggaagtgacctccactggaagccagacctagaccatggccaccccgtcttgggtcagtcatggaactgacgagcttctttgtgttacttcttccccgtggaaataactgtcgcctcgcagtaaggcaagtgagaagcagggaataaaagtgttgtgattaggaagttcaaagggagatactttcatagtgaactttgaaaagcttgtgtttatttcattagaatagaattcctgcaatcagacatgagggctctctttctgcttgggaggggggatggtcttgttacagaagtgactgctaatataatataaaattactcagctgggtttttaagttaattttttacattaaaatatagtttgaatttcttccgataaacggtaagcaataaatagctctacctcatctacccaaggtagaatttggcaacaaagcatcctttaggaagtttacaatgtgttaaagaagtgatatgtcttactcggaacaagaacatgttttgtaactctatgggttaaggtatgaacaagaattataggatataagaattttataagatagaatatcagttctacaaagcaagaattttatctgttgtgtcctcaatacctcagagagccag

SEQ ID NO: 4 - DNA, 1081 bp, Mus musculus
gctgttcacagggctaagataggactcttgcaagggatgcctctgctcttttacacagaggtcctgaaagaagaagccaaattccaacccaccttgtgactatgaagacgaaagggagagaaagtggggtcaggatttaggttagaacgcactaggtaactcggaatgttagactagaagatccacagaaaaatgactttagttatgtaatatgcttaattctcattctctctctctctctctctcgctcactcactcgcaagcaattcaaacccagcacttcataactgacaaatttctgtgtgtacatccaacacacacatacatatatccgtatgcagaatgtctttaggtgacgtgttcgattctcagaaaccatagcatcattcactttaagcacagctccggtcattttctctcattttcgtacagagaaataactgaaaaacccatgagatgagttcaccttcctcacgcccctcactaccttatttttatgcagtgggtagtacattcactttaaaattacgatatcttatacttaagggctgtttgttgctactatgaaatgatttttgtttttacttatttattttttactgtcgtgccagacttcaaaatccctgggtctaacacaagctcggagagtactctatttgaattatgcccttagtcctctctgacttttatacggtgccaacacagaaagtaaagtaccctgtgggggggggtgacaacctgctggcttaagtcgtatttcttattctaaagtagaaaactctagacaagaggaatgcagaagtcacatccagatttcattttctgtgaccagttttattctgcttttactacagatgagtcagggccccatcaaacccctccatgactggcaccacccatgaagtcattaggaaaaaaggccaaaatgtactttatagcatgacaaactgctctggacacacaagcattccactggtgataaattcgatctcattcttcagtgggttgccctacttccttggggttgcttaaagatctcgaaatatacctgagtccatgctcactctccacccacatgtgc

SEQ ID NO: 5 - DNA, 1143 bp, Mus musculus
atgcgagccgttcctctgcccctgagccggacagcaagcctcagccttggcttcttgctcctgctttctctctgcctggacccaggccaagccaaggagttgaagtttgtgacattggtgtttcggcatggagaccgaggtcccatcgagacctttcctaccgaccccattacagaatcctcgtggccacaaggatttggccaactcacccagtggggcatggaacagcactacgaacttggaagttatataaggaaaagatacggaagattcttgaacgacacctataagcatgatcagatttatatccggagcacagatgtggacaggactttgatgagtgctatgacaaaccttgcagccctgtttcctccagaggggatcagcatctggaatcctagactgctctggcagcccatcccagtgcacaccgtgtctctctctgaggatcggttgctgtacctgcctttcagagactgccctcgttttgaagaactcaagagtgagactttagaatctgaggaattcttgaagaggcttcatccatataaaagcttcctggacaccttgtcgtcgctgtcgggattcgatgaccaggatctttttggaatctggagtaaagtttatgaccctttattctgcgagagtgttcacaatttcaccttgccctcctgggccaccgaggacgccatgattaagttgaaagagctatcagaattatctctgctatcactttatggaattcacaagcagaaagagaaatctcgactccaagggggcgtcctggtcaatgaaatcctcaagaatatgaagcttgcaactcagccacagaagtataaaaagctggtcatgtattccgcacacgacactaccgtgagtggcctgcagatggcgctagatgtttataatggagttctgcctccctacgcttcttgccacatgatggaattgtaccatgataagggggggcactttgtggagatgtactatcggaatgagacccagaacgagccctacccactcacgctgccaggctgcacccacagctgccctctggagaagtttgcggagctactggacccggtgatctcccaggactgggccacggagtgtatggccacaagcagccaccaaggacggaat

SEQ ID NO: 6 - DNA, 1158 bp, Homo sapiens
agtggtagcagttcctcctaactcctgccagaaacagctctcctcaacatgagagctgcacccctcctcctggccagggcagcaagccttagccttggcttcttgtttctgctttttttctggctagaccgaagtgtactagccaaggagttgaagtttgtgactttggtgtttcggcatggagaccgaagtcccattgacacctttcccactgaccccataaaggaatcctcatggccacaaggatttggccaactcacccagctgggcatggagcagcattatgaacttggagagtatataagaaagagatatagaaaattcttgaatgagtcctataaacatgaacaggtttatattcgaagcacagacgttgaccggactttgatgagtgctatgacaaacctggcagccctgtttcccccagaaggtgtcagcatctggaatcctatcctactctggcagcccatcccggtgcacacagttcctctttctgaagatcagttgctatacctgcctttcaggaactgccctcgttttcaagaacttgagagtgagactttgaaatcagaggaattccagaagaggctgcacccttataaggattttatagctaccttgggaaaactttcaggattacatggccaggacctttttggaatttggagtaaagtctacgaccctttatattgtgagagtgttcacaatttcactttaccctcctgggccactgaggacaccatgactaagttgagagaattgtcagaattgtccctcctgtccctctatggaattcacaagcagaaagagaaatctaggctccaagggggtgtcctggtcaatgaaatcctcaatcacatgaagagagcaactcagataccaagctacaaaaaactcatcatgtattctgcgcatgacactactgtgagtggcctacagatggcgctagatgtttacaacggactccttcctccctatgcttcttgccacttgacggaattgtactttgagaagggggagtactttgtggagatgtactatcggaatgagacgcagcacgagccgtatcccctcatgctacctggctgcagccccagctgtcctctggagaggtttgctgagctggttggccctgtgatccctcaagactggtccacg